SEQUENCE LISTING (15 sequences)

SEQ ID NO: 1 - DNA, 6493 bp, Homo sapiens
  source 1..6493: mol_type = genomic DNA
agagcggagaagagcgagcaggggagagcgagaccagttttaaggggaggaccggtgcgagtgaggcagccccgaggctctgctcgcccaccacccaatcctcgcctcccttctgctccaccttctctctctgccctcacctctcccccgaaaaccccctatttagccaaaggaaggaggtcaggggaacgctctcccctccccttccaaaaaacaaaaacagaaaaacctttttccaggccggggaaagcaggagggagaggggccgccgggctggccatggagctgctgtgccacgaggtggacccggtccgcagggccgtgcgggaccgcaacctgctccgagacgaccgcgtcctgcagaacctgctcaccatcgaggagcgctaccttccgcagtgctcctacttcaagtgcgtgcagaaggacatccaaccctacatgcgcagaatggtggccacctggatgctggaggtctgtgaggaacagaagtgcgaagaagaggtcttccctctggccatgaattacctggaccgtttcttggctggggtcccgactccgaagtcccatctgcaactcctgggtgctgtctgcatgttcctggcctccaaactcaaagagaccagcccgctgaccgcggagaagctgtgcatttacaccgacaactccatcaagcctcaggagctgctggagtgggaactggtggtgctggggaagttgaagtggaacctggcagctgtcactcctcatgacttcattgagcacatcttgcgcaagctgccccagcagcgggagaagctgtctctgatccgcaagcatgctcagaccttcattgctctgtgtgccaccgactttaagtttgccatgtacccaccgtcgatgatcgcaactggaagtgtgggagcagccatctgtgggctccagcaggatgaggaagtgagctcgctcacttgtgatgccctgactgagctgctggctaagatcaccaacacagacgtggattgtctcaaagcttgccaggagcagattgaggcggtgctcctcaatagcctgcagcagtaccgtcaggaccaacgtgacggatccaagtcggaggatgaactggaccaagccagcacccctacagacgtgcgggatatcgacctgtgaggatgccagttgggccgaaagagagagacgcgtccataatctggtctcttcttctttctggttgtttttgttctttgtgttttagggtgaaacttaaaaaaaaaattctgcccccacctagatcatatttaaagatcttttagaagtgagagaaaaaggtcctacgaaaacggaataataaaaagcatttggtgcctatttgaagtacagcataagggaatcccttgtatatgcgaacagttattgtttgattatgtaaaagtaatagtaaaatgcttacaggaaaacctgcagagtagttagagaatatgtatgcctgcaatatgggaacaaattagaggagacttttttttttcatgttatgagctagcacatacacccccttgtagtataatttcaaggaactgtgtacgccatttatggcatgattagattgcaaagcaatgaactcaagaaggaattgaaataaggagggacatgatggggaaggagtacaaaacaatctctcaacatgattgaaccatttgggatggagaagcacctttgctctcagccacctgttactaagtcaggagtgtagttggatctctacattaatgtcctcttgctgtctacagtagctgctacctaaaaaaagatgttttattttgccagttggacacaggtgattggctcctgggtttcatgttctgtgacatcctgcttcttcttccaaatgcagttcattgcagacaccaccatattgctatctaatggggaaatgtagctatgggccataaccaaaactcacatgaaacggaggcagatggagaccaagggtgggatccagaatggagtcttttctgttattgtatttaaaagggtaatgtggccttggcatttcttcttagaaaaaaactaatttttggtgctgattggcatgtctggttcacagtttagcattgttataaaccattccattcgaaaagcactttgaaaaattgttcccgagcgatagatgggatggtttatgcaagtcatgctgaatactcctcccctcttctcttttgccccctcccttcctgcccccagtctgggttactcttcgcttctggtatctggcgttctttggtacacagttctggtgttcctaccaggactcaagagacaccccttcctgctgacattcccatcacaacattcctcagacaagcctgtaaactaaaatctgttaccattctgatggcacagaaggatcttaattcccatctctatacttctcctttggacatggaaagaaaagttattgctggtgcaaagatagatggctgaacatcagggtgtggcattttgttcccttttccgtttttttttttttattgttgttgttaattttattgcaaagttgtattcagcgtacttgaatttttcttcctctccacttcttagaggcattcagttagcaaagaggttggagcaacaacttttttttttttttttgcacaattgtaattgacaggtaatgaagctatttgttaaaatatttgcctttttaagtaaaaaagaaaaatcagaacagggctatttgaagaattattttatacacagattctgccttgtttcatagtatgagggttgaagacggaaaacaatctaagggtctctcatttttttaattttgttttgttcagtttggtttttttttttttttgcgctgctaagaagctaaagtcatccatccttattcacgttgacagtacctagctgtaatgtttcacagagtgtgctgctattttataaacatttttataatatattattttactgcttaaattccaagtcctgaagtagatggttgagatatgagttcttcgtactggaaaagcccttccgtagtttgttttcttctggtagcatattcatggttgtttttttttttcttttttggttttttggttttttttttttcctctgatcacattcttcaaagacggagtattctttacctcaggtttactggacaaaatcaataactacaaaaggcaatgattcacgcttttgttttcataatacctcacaaccgtacagtttctgcttgggagcccattcgcatgaggaatacagaagcagtgtgagcagggctgactccctctcaggtggaaggcagggcggtctcactcccagggacctttttggtcatggaggccatcgggctcccagttagaccctggtatcctcatcatgatggaaaaaatacattgaaccaagggatcctccctccccttcaaggcagacgttcagtacaaacatttatgcggtaggctcagatgtcgtaatttgcacttaggtaccaggtgtcaggaaacagactaaaaagaattccaccaggctgtttggagatcctcatcttggagctttttcaaaagcggggcttcatctgcaaagggccctttcatcttgaagtttttcccctccgtctttcccctcccctggcatggacaccttgtgtttaggatcatctctgcaggtttcctaggtctgaatctgcgagtagatgaacctgcagcaagcagcgtttatggtgcttccttctccctcctctgtctcaaactgcgcaggcaagcactatgcaagcccaggccctctgctgagcggtactaaacggtcgggttttcaatcacactgaattggcaggataagaaaaataggtcagataagtatgggatgatagttgaagggaggtgaagaggctgcttctctacagaggtgaaattccagatgagtcagtctcttgggaagtgtgtttagaagggttcaggactttgtgagttagcatgaccctaaaattctaggggatttctggtgggacaatgggtggtgaattctgaagttttggagagggaagtggagcagccagcaagtaagctagccagagttttctcaagagccagctttgctcagcacactctcctgggccccaaggagtcccacggaatggggaaagcgggaaccctggagttcttgggaatcttggagcctaaagagaaaccgaggtgcaaattcatttcatggtgactgacccttgagcttaaacagaagcagcaaatgaaagaaccggacaaataaggaagggcacaagcctacccgactctatttacagtctgtaactttccactcttcctgtagtcccgaggcccctgggtccttctagcttttctctttcccatccttggggccttgtgtgatgatgggtgtggggctgccgatgggaaagtcgggggttgttaggcttttctgcctgctcctgcttaaacacaagaaggaatcctggattttgccctctccttagctcttagtctctttggtaggagttttgttccagaggagctctcccccttggatttgaacttgctctttttgttgttgttgttctttctcttctttttcttacctcccactaaaggggttccaaattatcctggtctttttctaccttgttgtgtttctatctcgtctttacttccatctgtttgtttttttctccatcagtgggggccgagttgttcccccagcctgccaaattttgatccttcccctcttttggccaaatcctagggggaagaaatcctagtatgccaaaaatatatgctaagcataattaaactccatgcgggtccataacagccaagaagcctgcaggagaaagccaagggcagttccctccgcagaacaccccatgcgtgctgagaggcgagctccttgaagaaggggctgttcttccaggaggccttattttgaactgcctcaggaccccactggagagcacagcatgccttactactgggtcatccttggtctatgtgctctgtactggaggctctgttctgcctcttatcagccaggtcaggggcacacatggcttaagtgacaaagccagaggagaagacaaccctgacagcatcacgctgcatcccattgctagcaggattggcaactcttcagacggagctgcgcttccctgcagtctagcacctctagggcctctccagactgtgccctgggagctctgggactgaaaggttaagaacataaggcaggatcagatgactctctccaagagggcaggggaattttctctccatgggccacaggggacagggctgggagaagaaatagacttgcaccttatgtcatgtaaataattgattttctagttcaagaagataatattggtagtgtgggaattggaggtaggaaggggaggaagtctgagtaagccagttggcttctaagccaaaaggattcctctttgtttatctctgagacagtccaaccttgagaatagctttaaaagggaaattaatgctgagatgataaagtccccttaagccaacaaaccctctgtagctatagaatgagtgcaggtttctattggtgtggactcagagcaatttacaagagctgttcatgcagccatccatttgtgcaaaatagggtaagaagattcaagaggatatttattacttcctcataccacatggcttttgatgattctggattctaaacaacccagaatggtcatttcaggcacaacgatactacattcgtgtgtgtctgcttttaaacttggctgggctatcagaccctattctcggctcaggttttgagaagccatcagcaaatgtgtacgtgcatgctgtagctgcagcctgcatcccttcgcctgcagcctactttggggaaataaagtgccttactgactgtagccattacagtatccaatgtcttttgacaggtgcctgtccttgaaaaacaaagtttctatttttatttttaattggtttagttcttaactgctggccaactcttacatccccagcaaatcatcgggccattggattttttccattatgttcatcacccttatatcatgtacctcagatctctctctctctcctctctctcagttatgtagtttcttgtcttggacttttttttttcttttctttttctttttttttttgctttaaaacaagtgtgatgccatatcaagtccatgttattctctcacagtgtactctataagaggtgtgggtgtctgtttggtcaggatgttagaaagtgctgataagtagcatgatcagtgtatgcgaaaaggtttttaggaagtatggcaaaaatgttgtattggctatgatggtgacatgatatagtcagctgccttttaagaggtcttatctgttcagtgttaagtgatttaaaaaaataataacctgttttctgactagtttaaagatggatttgaaaatggttttgaatgcaattaggttatgctatttggacaataaactcaccttgacctaaa

SEQ ID NO: 2 - DNA, 870 bp, Homo sapiens
  source 1..870: mol_type = genomic DNA
atggagctgctgtgccacgaggtggacccggtccgcagggccgtgcgggaccgcaacctgctccgagacgaccgcgtcctgcagaacctgctcaccatcgaggagcgctaccttccgcagtgctcctacttcaagtgcgtgcagaaggacatccaaccctacatgcgcagaatggtggccacctggatgctggaggtctgtgaggaacagaagtgcgaagaagaggtcttccctctggccatgaattacctggaccgtttcttggctggggtcccgactccgaagtcccatctgcaactcctgggtgctgtctgcatgttcctggcctccaaactcaaagagaccagcccgctgaccgcggagaagctgtgcatttacaccgacaactccatcaagcctcaggagctgctggagtgggaactggtggtgctggggaagttgaagtggaacctggcagctgtcactcctcatgacttcattgagcacatcttgcgcaagctgccccagcagcgggagaagctgtctctgatccgcaagcatgctcagaccttcattgctctgtgtgccaccgactttaagtttgccatgtacccaccgtcgatgatcgcaactggaagtgtgggagcagccatctgtgggctccagcaggatgaggaagtgagctcgctcacttgtgatgccctgactgagctgctggctaagatcaccaacacagacgtggattgtctcaaagcttgccaggagcagattgaggcggtgctcctcaatagcctgcagcagtaccgtcaggaccaacgtgacggatccaagtcggaggatgaactggaccaagccagcacccctacagacgtgcgggatatcgacctgtga

SEQ ID NO: 3 - RNA, 870 nt, Homo sapiens
  source 1..870: mol_type = genomic RNA
atggagctgctgtgccacgaggtggacccggtccgcagggccgtgcgggaccgcaacctgctccgagacgaccgcgtcctgcagaacctgctcaccatcgaggagcgctaccttccgcagtgctcctacttcaagtgcgtgcagaaggacatccaaccctacatgcgcagaatggtggccacctggatgctggaggtctgtgaggaacagaagtgcgaagaagaggtcttccctctggccatgaattacctggaccgtttcttggctggggtcccgactccgaagtcccatctgcaactcctgggtgctgtctgcatgttcctggcctccaaactcaaagagaccagcccgctgaccgcggagaagctgtgcatttacaccgacaactccatcaagcctcaggagctgctggagtgggaactggtggtgctggggaagttgaagtggaacctggcagctgtcactcctcatgacttcattgagcacatcttgcgcaagctgccccagcagcgggagaagctgtctctgatccgcaagcatgctcagaccttcattgctctgtgtgccaccgactttaagtttgccatgtacccaccgtcgatgatcgcaactggaagtgtgggagcagccatctgtgggctccagcaggatgaggaagtgagctcgctcacttgtgatgccctgactgagctgctggctaagatcaccaacacagacgtggattgtctcaaagcttgccaggagcagattgaggcggtgctcctcaatagcctgcagcagtaccgtcaggaccaacgtgacggatccaagtcggaggatgaactggaccaagccagcacccctacagacgtgcgggatatcgacctgtga

SEQ ID NO: 4 - protein, 289 aa, Homo sapiens
MELLCHEVDPVRRAVRDRNLLRDDRVLQNLLTIEERYLPQCSYFKCVQKDIQPYMRRMVATWMLEVCEEQKCEEEVFPLAMNYLDRFLAGVPTPKSHLQLLGAVCMFLASKLKETSPLTAEKLCIYTDNSIKPQELLEWELVVLGKLKWNLAAVTPHDFIEHILRKLPQQREKLSLIRKHAQTFIALCATDFKFAMYPPSMIATGSVGAAICGLQQDEEVSSLTCDALTELLAKITNTDVDCLKACQEQIEAVLLNSLQQYRQDQRDGSKSEDELDQASTPTDVRDIDL

SEQ ID NO: 5 - DNA, 18 bp, synthetic construct
  source 1..18: mol_type = other DNA
  modified_base 1: mod_base = OTHER; note = Locked nucleic acid with phosphorothioate linkage
  modified_base 2: mod_base = OTHER; note = Locked nucleic acid with phosphorothioate linkage
  modified_base 3: mod_base = OTHER; note = Locked nucleic acid with phosphorothioate linkage
  modified_base 4: mod_base = OTHER; note = Locked nucleic acid with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Locked nucleic acid with phosphorothioate linkage
  modified_base 16: mod_base = OTHER; note = Locked nucleic acid with phosphorothioate linkage
  modified_base 17: mod_base = OTHER; note = Locked nucleic acid with phosphorothioate linkage
  modified_base 18: mod_base = OTHER; note = Locked nucleic acid
gcgcatgtagggttggat

SEQ ID NO: 6 - DNA, 18 bp, synthetic construct
  source 1..18: mol_type = other DNA
  modified_base 1: mod_base = OTHER; note = Locked nucleic acid with phosphorothioate linkage
  modified_base 2: mod_base = OTHER; note = Locked nucleic acid with phosphorothioate linkage
  modified_base 3: mod_base = OTHER; note = Locked nucleic acid with phosphorothioate linkage
  modified_base 4: mod_base = OTHER; note = Locked nucleic acid with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Locked nucleic acid with phosphorothioate linkage
  modified_base 16: mod_base = OTHER; note = Locked nucleic acid with phosphorothioate linkage
  modified_base 17: mod_base = OTHER; note = Locked nucleic acid with phosphorothioate linkage
  modified_base 18: mod_base = OTHER; note = Locked nucleic acid
gcgcatgtacggctggat

SEQ ID NO: 7 - DNA, 18 bp, synthetic construct
  source 1..18: mol_type = other DNA
  modified_base 1: mod_base = OTHER; note = Locked nucleic acid with phosphorothioate linkage
  modified_base 2: mod_base = OTHER; note = Locked nucleic acid with phosphorothioate linkage
  modified_base 3: mod_base = OTHER; note = Locked nucleic acid with phosphorothioate linkage
  modified_base 4: mod_base = OTHER; note = Locked nucleic acid with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Locked nucleic acid with phosphorothioate linkage
  modified_base 16: mod_base = OTHER; note = Locked nucleic acid with phosphorothioate linkage
  modified_base 17: mod_base = OTHER; note = Locked nucleic acid with phosphorothioate linkage
  modified_base 18: mod_base = OTHER; note = Locked nucleic acid
aggtaattcatggccaga

SEQ ID NO: 8 - DNA, 20 bp, synthetic construct
  source 1..20: mol_type = other DNA
gtcgccccagactacgataa

SEQ ID NO: 9 - DNA, 20 bp, synthetic construct
  source 1..20: mol_type = other DNA
gccaagaccaccacaactct

SEQ ID NO: 10 - DNA, 20 bp, synthetic construct
  source 1..20: mol_type = other DNA
aagaacacgagccaacaagc

SEQ ID NO: 11 - DNA, 20 bp, synthetic construct
  source 1..20: mol_type = other DNA
taccatacccagtgccatca

SEQ ID NO: 12 - DNA, 21 bp, synthetic construct
  source 1..21: mol_type = other DNA
cagaaggacatccagccgtac

SEQ ID NO: 13 - DNA, 19 bp, synthetic construct
  source 1..19: mol_type = other DNA
tcgggactccagccaagaa

SEQ ID NO: 14 - DNA, 21 bp, synthetic construct
  source 1..21: mol_type = other DNA
ctttgaccgacacctgtttgc

SEQ ID NO: 15 - DNA, 22 bp, synthetic construct
  source 1..22: mol_type = other DNA
tggtggacaggatgttgtggtt

(19) United States
(12) Patent Application Publication
(10) Pub. No.: US 2026/0166072 A1
(43) Pub. Date: Jun. 18, 2026

(54) OLIGONUCLEOTIDE-BASED THERAPEUTICS TARGETING CYCLIN D2 FOR THE TREATMENT OF HEART FAILURE

(71) Applicants: GenKardia Inc., Dover, DE (US); THE CHINESE UNIVERSITY OF HONG KONG, Hong Kong (CN)

(72) Inventors: David C. Zebrowski, Dover, DE (US); Ngar Yun Ellen Poon, Hong Kong (CN); Fang Meng, Hong Kong (CN); Ka Shing Maxwell Kwok, Hong Kong (CN)

(21) Appl. No.: 19/127,121

(22) PCT Filed: Nov. 6, 2023
(86) PCT No.: PCT/US2023/078835

§ 371 Date: May 4, 2025

Related U.S. Application Data
(60) Provisional application No. 63/382,484, filed on Nov. 4, 2022.

(51) Int. Cl. A61K 31/712; A61K 31/7115; A61K 31/7125; A61P 9/00; A61P 9/04; C12N 15/113

(52) U.S. Cl. CPC A61K 31/712; A61K 31/7115; A61K 31/7125; A61P 9/00; A61P 9/04; C12N 15/113; C12N 2310/11

(57) ABSTRACT

This disclosure relates to methods and compositions for treating heart failure (e.g., reducing or inhibiting cardiomyocyte hypertrophy or by increasing utilization of fatty acids as an energy source and Fatty Acid oxidation in cardiomyocytes or by reducing lipid accumulation in, and lipotoxicity of, cardiomyocytes) where the method includes administering to the patient a therapeutically effective amount of an oligonucleotide-based therapeutic comprising a polynucleotide sequence that is at least partially complementary to a nucleic acid sequence with a Cyclin D2 (CCND2) transcript.

### 1. CROSS-REFERENCE TO RELATED APPLICATIONS

This application claims the benefit of U.S. Provisional Patent Application No. 63/382,484, filed Nov. 4, 2022, which is hereby incorporated in its entirety by reference.

### 2. SEQUENCE LISTING

The instant application contains a Sequence Listing with 15 sequences, which has been submitted via Patent Center and is hereby incorporated by reference in its entirety. Said XML copy, created on Oct. 19, 2023, is named 50800_WO_002WO_CRF_sequencelisting, and is 52 kilobytes in size.

### 3. BACKGROUND

Heart failure is a condition that results from an inability of the heart to pump blood efficiently to meet the body's demands. Heart failure is a leading cause of death in industrialized nations. Furthermore, the 5-year survival index upon heart failure diagnosis is 50%—worse than many, cancer diagnoses. To date, there is no cure for heart failure and treatment options are limited. Novel disease altering strategies, targets, and therapeutic modalities are required to treat heart failure.

Various pathologic stressors (e.g., neurohumoral activation, increased cardiac mechanical load (e.g., pressure overload), cytokines, and co-morbidities such as arterial hypertension, chronic kidney disease, obesity, and diabetes) induce an increase in cardiac and cardiomyocyte size (termed “pathologic hypertrophy”). Similarly, congenital heart disease, various cardiomyopathies (e.g. those resulting from mutations in sarcomere genes), as well as myocardial infarction can induce pathologic hypertrophy. Pathologic hypertrophy, in its initial phase, is associated with normal cardiac function. However, chronic pathologic stress, and persistent hypertrophy, ultimately is associated with reduced cardiac function and heart failure (Ruppert, M., Am. J. Physiol. Heart Circ. Physiol., 311(3): p. H592-603 (2016); Manyari, D. E., Framingham Heart Study. N. Engl. J. Med., 323(24): p. 1706-7 (1990)).

Suppression of cardiac hypertrophy, even though the pathological stress is persistent (e.g., pressure-overload), mitigates heart failure development and/or progression. Recent studies suggest that suppression of hypertrophy, even though external stressors persist, represents a therapeutic target to treat heart failure (Frey, N., et al., Circulation, 109(13): p. 1580-9 (2004)). While numerous targets have been identified that regulate cardiac hypertrophy, many of these targets are not druggable. Identifying novel targets and/or target strategies that are druggable and suppress cardiomyocyte pathologic hypertrophy presents an opportunity to treat heart failure.

Additionally, recent studies suggest that metabolic remodeling (i.e. a shift from FAO to Glycolysis) contributes to the pathogenesis of heart failure. These observations, and others, have implicated metabolic remodeling to be a maladaptive event. Thus, inhibiting or reversing metabolic remodeling is believed to be a therapeutic strategy to treat heart failure. However, identifying novel targets and/or target strategies that are druggable and modulate metabolic remodeling presents remains an unmet need.

The inability to utilize fatty acids as an energy source can result in an accumulation of fatty acids (FA) within the cytosol of the cell. There is increasing evidence that FA-uptake from the extracellular environment, under conditions of suppressed FAO, can result in lipotoxicity (i.e. cell death due to accumulation of excess lipids). Lipid overload in the cell has been reported to involve endoplasmic reticulum stress, alterations in autophagy, de novo ceramide synthesis, and oxidative stress. Furthermore, lipotoxicity has been implicated as a contributing factor in cardiac dysfunction and the development of heart failure in animal models of heart failure and in humans with heart failure (Abdurrachim D, et. al., Cardiovascular Research (2015) 106, 194-205; Konstantinos Drosatos and P. Christian Schulze, Curr. Heart Fail Rep., 2013, June; 10(2): 109-121; (Leggat et al., Clinical Science, Oct. 13, 2021). Thus, reducing lipid accumulation in cardiomyocytes and inhibiting lipotoxicity related cardiomyocyte death is believed to be a therapeutic strategy to treat heart failure. However, identifying novel targets and/or target strategies that are druggable and reduce lipid accumulation in cardiomycotes, thereby inhibiting lipotoxicity, remains an unmet clinical need.

### 4. SUMMARY

As provided herein, reducing or inhibiting Cyclin D2 expression (i) suppresses cardiomyocyte pathologic hypertrophy, (ii) promotes fatty acid oxidation, and (iii) reduces fatty acid accumulation in cardiomyocytes and fatty acid induced cardiomyocyte lipotoxicity thereby treating heart failure.

The present disclosure relates to methods of treating heart failure where the method includes administering to the patient a therapeutically effective amount of an oligonucleotide-based therapeutic comprising a polynucleotide sequence that is at least partially complementary to a nucleic acid sequence with a Cyclin D2 (CCND2) transcript. For example, Applicant showed that reducing or inhibiting Cyclin D2 expression in cardiomyocytes using an oligonucleotide-based therapeutic (e.g., an ASO or a siRNA targeting Cyclin D2) promoted (i) suppression of hypertrophic growth, (ii) preservation of metabolic/FAO capacity under pathological stimuli (e.g., Angiotensin II (ANGII) or Isoproterenol (ISO)), and (iii) suppressed fatty acid-induced lipotoxicity (e.g., cell death).

Overall, provided herein are data illustrating that treating heart failure by reducing or inhibiting expression of Cyclin D2 provides a superior therapeutic modality by uniquely and specifically targeting the underlying mechanisms driving heart failure.

In one aspect, provided herein is a method for treating a patient having, or at risk of having, heart failure, comprising: administering to the patient a therapeutically effective amount of an oligonucleotide-based therapeutic comprising a polynucleotide sequence that is at least partially complementary to a nucleic acid sequence within a Cyclin D2 (CCND2) transcript, wherein Cyclin D2 expression in the patient is inhibited or reduced after the administration, thereby treating the patient having, or at risk of having, heart failure. In some embodiments, heart failure comprises cardiac hypertrophy.

In another aspect, provided herein is a method for reducing or preventing hypertrophy of a plurality of cardiomyocytes, comprising: contacting the cardiomyocytes with an effective amount of an oligonucleotide-based therapeutic comprising a polynucleotide sequence that is at least partially complementary to a nucleic acid sequence within a Cyclin D2 (CCND2) transcript, wherein Cyclin D2 expression in the cardiomyocytes is inhibited or reduced after contacting the cardiomyocytes, thereby reducing or preventing hypertrophy of a plurality of cardiomyocytes.

In some embodiments, the reducing or inhibiting of Cyclin D2 expression reduces or prevents hypertrophy of the plurality of cardiomyocytes. In some embodiments, the plurality of cardiomyocytes are those of the heart of a patient. In some embodiments, the patient has, or is at risk of having, heart failure, preferably wherein the heart failure comprises cardiac hypertrophy.

In another aspect, provided herein is a method for maintaining or increasing utilization of fatty-acid oxidation in a cardiomyocyte, comprising: contacting the cardiomyocyte with an effective amount of an oligonucleotide-based therapeutic comprising a polynucleotide sequence that is at least partially complementary to a nucleic acid sequence within a Cyclin D2 (CCND2) transcript, wherein Cyclin D2 expression is inhibited or reduced after contacting the cardiomyocyte, thereby maintaining or increasing utilization of fatty acid oxidation.

In some embodiments, reducing or inhibiting Cyclin D2 expression increases the cardiomyocyte's utilization of fatty-acid oxidation. In some embodiments, the cardiomyocyte is in the heart of a patient.

In another aspect, provide herein is a method for reducing or preventing fatty acid accumulation and fatty acid-induced lipotoxicity in a cardiomyocyte, comprising: contacting the cardiomyocyte with an effective amount of an oligonucleotide-based therapeutic comprising a polynucleotide sequence that is at least partially complementary to a nucleic acid sequence within a Cyclin D2 (CCND2) transcript, wherein Cyclin D2 expression is inhibited or reduced after contacting the cardiomyocyte, thereby reducing lipid accumulation and lipotoxicity.

In some embodiments, reducing or inhibiting Cyclin D2 expression reduces lipid accumulation and lipotoxicity in the cardiomyocyte. In some embodiments, the cardiomyocyte is in the heart of a patient.

In another aspect, provided herein is a method for inhibiting or reducing Cyclin D2 expression in a cell, comprising: contacting the cell with an effective amount of an oligonucleotide-based therapeutic comprising a polynucleotide sequence that is at least partially complementary to a nucleic acid sequence within a Cyclin D2 (CCND2) transcript, wherein Cyclin D2 expression is inhibited or reduced after contacting the cell with the polynucleotide sequence.

In some embodiments, the cell is a cardiomyocyte. In some embodiments, the cardiomyocyte is in the heart of a patient.

In another aspect, this disclosure provides an oligonucleotide-based therapeutic for inhibiting or reducing Cyclin D2 expression in a cell, comprising a polynucleotide sequence that is at least partially complementary to a nucleic acid sequence within a Cyclin D2 (CCND2) transcript. In some embodiments, the oligonucleotide-based therapeutic is selected from: an anti-sense oligonucleotide (ASO), a short interfering RNA (siRNA), a double-stranded ribonucleic acid (dsRNA), a single-stranded siRNAs (ssRNAs), a small temporal RNA (stRNA), a short hairpin RNA (shRNA), a locked nucleic acid (LNA), an antagomirs aptamer, a peptide nucleic acid (PNA), and a microRNA (miRNA).

In some embodiments, the polynucleotide sequence comprises a sequence having at least about 80%, at least about 85%, at least about 90%, at least about 95%, or 100% complementarity to the nucleic acid sequence within the Cyclin D2 transcript.

In some embodiments, the Cyclin D2 transcript is selected from a sequence of SEQ ID NO: 1-3.

In some embodiments, Cyclin D2 expression (protein/transcript) is reduced by at least 25% (e.g., at least 30%, at least 35%, at least 40%, at least 45%, at least 50%, at least 55%, at least 60%, at least 65%, at least 70%, at least 75%, at least 80%, at least 90%, at least 95% or 100%) as compared to Cyclin D2 expression (protein/transcript) in a patient not treated or cell not contacted with the oligonucleotide-based inhibitor.

In some embodiments, the oligonucleotide-based therapeutic is selected from: an anti-sense oligonucleotide (ASO), a short interfering RNA (siRNA), a single-stranded siRNAs (ssRNAs), a small temporal RNA (stRNA), a short hairpin RNA (shRNA), a locked nucleic acids (LNA), an antagomirs aptamer, a peptide nucleic acids (PNA), and a microRNA (miRNA).

In some embodiments, the oligonucleotide-based therapeutic is an anti-sense oligonucleotide (ASO).

In some embodiments, the ASO comprises a polynucleotide sequence having at least about 80%, at least about 85%, at least about 90%, at least about 95%, or about 100% complementarity to the nucleic acid sequence within the Cyclin D2 transcript.

In some embodiments, the ASO comprises the sequence selected from SEQ ID NOs: 5-7.

In some embodiments, the ASO has a sequence having at least 80% sequence identity to SEQ ID NO: 5. In some embodiments, the ASO has a sequence having at least 85% sequence identity to SEQ ID NO: 5. In some embodiments, the ASO has a sequence having at least 90% sequence identity to SEQ ID NO: 5. In some embodiments, the ASO has a sequence having at least 95% sequence identity to SEQ ID NO: 5.

In some embodiments, the ASO has a sequence having at least 80% sequence identity to SEQ ID NO: 7. In some embodiments, the ASO has a sequence having at least 85% sequence identity to SEQ ID NO: 7. In some embodiments, the ASO has a sequence having at least 90% sequence identity to SEQ ID NO: 7. In some embodiments, the ASO has a sequence having at least 95% sequence identity to SEQ ID NO: 7.

In some embodiments, the ASO is selected from a gapmer, a blockmer, a mixmer, a headmer, a tailmer, a totalmer, a morpholino, a steric blocking ASO, and a CpG ASO.

In some embodiments, the ASO is a gapmer comprising a 5′ flanking region and a 3′ flanking region.

In some embodiments, the ASO comprises at least one non-naturally occurring nucleoside.

In some embodiments, the non-naturally occurring nucleoside is a sugar modified nucleoside.

In some embodiments, the non-naturally occurring nucleoside comprises a 2′-O-alkyl-RNA; 2′-O-methyl RNA (2′-OMe); 2′-alkoxy-RNA; 2′-O-methoxyethyl-RNA (2′-MOE); 2′-amino-DNA; 2′-fluoro-RNA; 2′-fluoro-DNA; arabino nucleic acid (ANA); 2′-fluoro-ANA; or bicyclic nucleoside analog (LNA).

In some embodiments, the sugar modified nucleoside is an affinity enhancing 2′ sugar modified nucleoside.

In some embodiments, the affinity enhancing 2′ sugar modified nucleoside is an LNA. In some embodiments, the LNA is a constrained ethyl nucleoside (cEt), 2′,4′-constrained 21-O-methoxyethyl (cMOE), α-L-LNA, β-D-LNA, 2′-0,4′-C-ethylene-bridged nucleic acids (ENA), amino-LNA, oxy-LNA, thio-LNA, or any combination thereof. In some embodiments, the LNA is β-D-LNA or oxy-LNA.

In some embodiments, the ASO comprises one or more 5′-methyl-cytosine nucleobases.

In some embodiments, the contiguous nucleotide sequence comprises one or more modified internucleoside linkages. In some embodiments, the one or more modified internucleoside linkages is a phosphorothioate linkage.

In some embodiments, the gapmer comprises at least one LNA. In some embodiments, the gapmer comprises at least one LNA in the 5′ flanking region. In some embodiments, the gapmer comprises at least one LNA in the 3′ flanking region. In some embodiments, the gapmer comprises at least one LNA in the 5′ flanking region and at least one LNA in the 3′ flanking region.

In some embodiments, the ASO is conjugated to a non-nucleic acid moiety, wherein the non-nucleic acid moiety comprises a fatty acid, lipid, peptide, protein, antibody, or nanoparticle.

In some embodiments, the ASO is capable of reducing Cyclin D2 protein and/or Cyclin D2 transcript expression in the patient, cardiomyocyte, or cell by at least about 20% compared to a corresponding patient, cardiomyocyte, or cell not exposed to the ASO.

In some embodiments, the oligonucleotide-based therapeutic is a double-stranded ribonucleic acid (dsRNA) molecule.

In some embodiments, the dsRNA is a small interfering (siRNA). In some embodiments, the siRNA comprises (a) a duplex region and (b) optionally one or two overhang regions, wherein each overhang region is six or fewer nucleotides, wherein the duplex region comprises a sense and an antisense region each comprising between 15 and 30 nucleotides,

In some embodiments, the siRNA comprises a sense strand and an antisense strand, wherein the antisense strand comprises the polynucleotide sequence that is at least partially complementary to the nucleic acid sequence within a Cyclin D2 (CCND2) transcript.

In some embodiments, the polynucleotide sequence of the antisense strand has at least 80% complementarity to a contiguous sequence of at least 18 nucleotides of the nucleic acid sequence of the Cyclin D2 transcript (SEQ ID NO: 1). In some embodiments, the polynucleotide sequence of the antisense strand has at least 85% complementarity to a contiguous sequence of at least 18 nucleotides of the nucleic acid sequence of the Cyclin D2 transcript (SEQ ID NO: 1). In some embodiments, the polynucleotide sequence of the antisense strand has at least 85% complementarity to a contiguous sequence of at least 18 nucleotides of the nucleic acid sequence of the Cyclin D2 transcript (SEQ ID NO: 1). In some embodiments, the polynucleotide sequence of the antisense strand has at least 85% complementarity to a contiguous sequence of at least 18 nucleotides of the nucleic acid sequence of the Cyclin D2 transcript (SEQ ID NO: 1).

In some embodiments, the sense strand, the antisense strand, or both comprise at least one chemical modification. In some embodiments, the at least one chemical modification is selected from at least one modified nucleotide, a substitution of a nucleotides with other another nucleotide, a non-natural phosphodiester linkage, or a combination thereof.

In some embodiments, the at least one chemical modification comprises at least one modified nucleotide. In some embodiments, the at least one modified nucleotide is selected from: a 2′-O-methyl modified nucleotide, a 5′-phosphorothioate group modified nucleotide, a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, a non-natural base comprising nucleotide, and a terminal nucleotide linked to a cholesteryl derivative or dodecanoic acid bisdecylamide group.

In some embodiments, the at least one modified nucleotide is 2′-O-methyl modified nucleotide.

In some embodiments, at least 5 of the nucleotides of the sense and antisense strands are modified at the 2′position of the ribose sugars of the nucleotides.

In some embodiments, at least 10 of the nucleotides of the sense and antisense strands are modified at the 2′position of the ribose sugars of the nucleotides.

In some embodiments, at least one chemical modification comprises at least one non-natural phosphodiester linkage. In some embodiments, the sense strand, antisense strand, or both comprise at least five non-natural phosphodiester linkages. In some embodiments, the non-natural phosphodiester linkages are selected from a phosphorothioate, a methylphosphonate, and a peptide.

In some embodiments, at least one chemical modification comprises a substitution of least one uracil nucleotide in the sense or antisense region with a 5-propynyluracil nucleotide.

In some embodiments, the at least one chemical modification comprises a substitution of least one uracil nucleotide in the sense or antisense region with a 5-methyluridine nucleotide.

In some embodiments, the at least one chemical modification comprises substitution of least one uracil nucleotide in the sense or antisense region with a deoxythymidine nucleotide.

In some embodiments, the at least one chemical modification comprises substitution of least one cytosine nucleotide in the sense or antisense region with a 5-methylcytosine nucleotide.

In some embodiments, the at least one chemical modification comprises substitution of least one adenine or guanine nucleotide in the sense or antisense region with an 8-bromoadenine or 8-bromoguanine nucleotide.

In some embodiments, the siRNA is conjugated to a non-nucleic acid molecule, wherein the non-nucleic acid molecule comprises a fatty acid, lipid, peptide, protein, antibody, or nanoparticle.

In some embodiments, the siRNA is capable of reducing Cyclin D2 protein and/or Cyclin D2 transcript expression in the patient, cardiomyocyte, or cell by at least about 20% compared to a corresponding patient, cardiomyocyte, or cell not exposed to the siRNA.

In some embodiments, the oligonucleotide-based therapeutic is administered (or contacted to the cell) in a pharmaceutically acceptable composition.

In some embodiments, the pharmaceutically acceptable composition is a liposomal composition.

### 5. BRIEF DESCRIPTION OF THE DRAWINGS

These and other features, aspects, and advantages of the present disclosure will become better understood with regard to the following description, and accompanying drawings, where:

FIGS. 1A-1B show the effect of an siRNA targeting Cyclin D2 in neonatal rat ventricular cardiomyocytes (isolated from 7-day old pups). FIG. 1A shows immunofluorescence images stained with an anti-Sarcomeric Alpha Actinin and DAPI. FIG. 1B shows effect of siRNA-mediated knockdown of Cyclin D2 in neonatal rat cardiomyocytes isolated from 7-day old pups on cell area (A.U.) (mean (solid line), n=3, >50 cardiomyocytes measured per n) for three groups: siRNA Scramble, ISO+siRNA Scramble, and ISO+siRNA Cyclin D2. The box and violin plot generated by an unpaired t test, shows Cyclin D2 siRNA significantly (p<0.05) suppresses ISO-induced increase in cell area.

FIGS. 2A-2B show the effect of an siRNA targeting Cyclin D2 in neonatal rat ventricular cardiomyocytes (isolated from 7-day old pups). FIG. 2A shows immunofluorescence images stained with an anti-Sarcomeric Alpha Actinin and DAPI. FIG. 2B shows effect of siRNA-mediated knockdown of Cyclin D2 in neonatal rat cardiomyocytes isolated from 7-day old pups on cell area (A.U.) (mean (solid line), n=3, >50 cardiomyocytes measured per n) for three groups: siRNA Scramble, ANGII+siRNA Scramble, and ANGII+siRNA Cyclin D2. The box and violin plot generated by an unpaired t test, shows Cyclin D2 siRNA significantly (p<0.05) suppresses ANGII-induced increase in cell area.

FIG. 3 shows an LNA gapmer antisense oligonucleotide (ASO) mediated suppression of Cyclin D2 expression in neonatal rat ventricular cardiomyocytes (isolated from 7-day old pups) on Cyclin D2 expression (RT-PCR) (mean with SEM, n=3, >50 cardiomyocytes measured per n) for three groups: Scramble ASO, ISO+Scramble ASO, and ISO+Cyclin D2 ASO.

FIG. 4 shows the effect of an LNA gapmer antisense oligonucleotide (ASO) targeting Cyclin D2 in neonatal rat ventricular cardiomyocytes (isolated from 7-day old pups) on Cyclin D2 expression (RT-PCR) (mean with SEM, n=3) for three groups: Scramble ASO, ANGII+Scramble ASO, and ANGII+Cyclin D2 ASO.

FIGS. 5A-5B shows the effect of an LNA gapmer antisense oligonucleotide (ASO) targeting Cyclin D2 in neonatal rat ventricular cardiomyocytes (isolated from 7-day old pups) (“P7-NVRMs”). FIG. 5A shows immunofluorescence images of P7-NVRMS stained with an anti-Sarcomeric Alpha Actinin and DAPI. FIG. 5B shows the effect of ASO-mediated knockdown of Cyclin D2 in neonatal rat cardiomyocytes isolated from 7-day old pups on cell area (A.U.) (mean (solid line), n=3, >50 cardiomyocytes measured per n) for three groups: Scramble ASO, ISO+Scramble ASO, and ISO+Cyclin D2 ASO. The box plot generated by an unpaired t test, shows Cyclin D2 ASO significantly (p<0.05) suppresses ISO-induced increase in cell area.

FIGS. 6A-6B show the effect of an LNA gapmer antisense oligonucleotide (ASO) targeting Cyclin D2 in neonatal rat ventricular cardiomyocytes (isolated from 7-day old pups) (“P7-NVRMs”). FIG. 6A shows immunofluorescence images of P7-NVRMs stained with an anti-Sarcomeric Alpha Actinin and DAPI. FIG. 6B shows the effect of ASO-mediated knockdown of Cyclin D2 in neonatal rat cardiomyocytes isolated from 7-day old pups on cell area (A.U.) (mean (solid line), n=3, >50 cardiomyocytes measured per n) for three groups: Scramble ASO, ANGII+Scramble ASO, and ANGII+Cyclin D2 ASO. The box plot generated by an unpaired t test, shows Cyclin D2 ASO significantly (p<0.05) suppresses ANGII-induced increase in cell area.

FIGS. 7A-7B show the effect of an LNA gapmer antisense oligonucleotide (ASO) targeting Cyclin D2 in human induced-pluripotent stem cell derived cardiomyocytes (hiPSC-CMs) (acquired from Cellular Dynamics International (aged to day 30)). FIG. 7A shows immunofluorescence images stained with an anti-Sarcomeric Alpha Actinin and DAPI. FIG. 7B shows the effect of ASO-mediated knockdown of Cyclin D2 on cell area (A.U.) (mean (solid line), n=3, >50 cardiomyocytes measured per n) for three groups: Scramble ASO, ANGII+Scramble ASO, and ANGII+Cyclin D2 ASO. The box and violin plot generated by an unpaired t test, shows Cyclin D2ASO significantly (p<0.05) suppresses ISO-induced increase in cell area.

FIGS. 7C-7D show the effect of an LNA gapmer antisense oligonucleotide (ASO) targeting Cyclin D2 in human induced-pluripotent stem cell derived cardiomyocytes (hiPSC-CMs) (acquired from Cellular Dynamics International (aged to day 30)). FIG. 7C shows immunofluorescence images stained with an anti-Sarcomeric Alpha Actinin and DAPI. FIG. 7D shows the effect of ASO-mediated knockdown of Cyclin D2 on cell area (A.U.) (mean (solid line), n=3, >50 cardiomyocytes measured per n) for three groups: Scramble ASO, ISO+Scramble ASO, and ISO+Cyclin D2 ASO. The box and violin plot generated by an unpaired t test, shows Cyclin D2ASO significantly (p<0.05) suppresses ISO-induced increase in cell area.

FIG. 8 shows relative gene expression (RT-PCR) (SEM (n=3)) of MCAD and MCPT1 in neonatal rat ventricular cardiomyocytes (isolated from 7-day old pups) for groups: ISO+siRNA Scramble, ISO+siRNA D1, ISO+siRNA D2, and ISO+siRNA D3.

FIG. 9 shows relative gene expression (RT-PCR) (SEM (n=3)) of MCAD and MCPT1 in neonatal rat ventricular cardiomyocytes (isolated from 7-day old pups) for groups: ANGII+siRNA Scramble, ANGII+siRNA D1, ANGII+siRNA D2, and ANGII+siRNA D3.

FIG. 10 shows relative gene expression (RT-PCR) (SEM (n=3)) of MCAD and MCPT1 in neonatal rat ventricular cardiomyocytes (isolated from 7-day old pups) for two groups: ISO+Scramble ASO, and ISO+Cyclin D2 ASO.

FIG. 11 shows relative gene expression (RT-PCR) (SEM (n=3)) of MCAD and MCPT1 in neonatal rat ventricular cardiomyocytes (isolated from 7-day old pups) for two groups: ANGII+Scramble ASO, and ANGII+Cyclin D2 ASO.

FIG. 12 shows relative gene expression (RT-PCR) (SEM (n=3)) of MCPT1 in hiPSC-derived cardiomyocytes acquired from Cellular Dynamics International (aged to day 30) for two groups: ISO+Scramble ASO, and ISO+Cyclin D2 ASO.

FIG. 13A shows for illustrative purposes only a protocol for Fatty Acid Oxidation (FAO) analysis via Seahorse.

FIG. 13B shows for illustrative purposes only a chart showing how to interpret data from the palmitate stress test generated by Seahorse assay.

FIG. 13C shows Oxygen Consumption Rate (OCR) using a palmitate stress test (where palmitate, a fatty acid, is the primary energy source in the media) in neonatal rat ventricular cardiomyocytes (isolated from 7-day old pups) for two groups: ISO+Scramble ASO. and ISO+Cyclin D2 ASO.

FIG. 13D shows Maximal Respiration in neonatal rat ventricular cardiomyocytes (isolated from 7-day old pups) for two groups: ISO with Scramble ASO, and ISO with Cyclin D2 ASO.

FIG. 13E shows Spare Reserve Capacity in neonatal rat ventricular cardiomyocytes (isolated from 7-day old pups) for two groups: ISO with Scramble ASO, and ISO with Cyclin D2 ASO.

FIG. 14A shows for illustrative purposes only a protocol for Fatty Acid Oxidation (FAO) analysis via Seahorse.

FIG. 14B shows for illustrative purposes only a chart showing how to interpret data from the palmitate stress test generated by Seahorse assay.

FIG. 14C shows Oxygen Consumption Rate (OCR) using a palmitate stress test (where palmitate, a fatty acid, is the primary energy source in the media) in neonatal rat cardiomyocytes (isolated from 7-day old pups) for two groups: ANGII+Scramble ASO, and ANGII+Cyclin D2 ASO.

FIG. 14D shows Maximal Respiration in neonatal rat cardiomyocytes (isolated from 7-day old pups) for two groups: ANGII+Scramble ASO, and ANGII+Cyclin D2 ASO.

FIG. 14E shows Spare Reserve Capacity in neonatal rat cardiomyocytes (isolated from 7-day old pups) for two groups: ANGII+Scramble ASO, and ANGII+Cyclin D2 ASO.

FIG. 15A shows a protocol for a lipid accumulation test where lipids were cultured with cells for 36 hours.

FIG. 15B shows the effect of an LNA gapmer antisense oligonucleotide (ASO) targeting Cyclin D2 in human induced-pluripotent stem cell derived cardiomyocytes (hiPSC-CMs) using BODIPY-Palmitate (BODIPY C16) (i.e., a fluorescently (488) tagged fatty acid). FIG. 15B shows images for four groups: Scramble ASO, ANGII+Scramble ASO, ANGII+Cyclin D2 ASO (SEQ ID NO: 5), ANGII+Cyclin D2 ASO (SEQ ID NO: 7).

FIG. 15C shows the effect of an LNA gapmer antisense oligonucleotide (ASO) targeting Cyclin D2 in human induced-pluripotent stem cell derived cardiomyocytes (hiPSC-CMs) (acquired from Cellular Dynamics International (aged to day 30)) accumulation of fatty acids (mean (solid line), n=3, >50 cardiomyocytes measured per n) for four groups: Scramble ASO, ANGII+Scramble ASO, ANGII+Cyclin D2 ASO (SEQ ID NO: 5), ANGII+Cyclin D2 ASO (SEQ ID NO: 7). The box and violin plot generated by an unpaired t test, shows Cyclin D2 ASOs significantly (p<0.05) suppresses ANGII-induced increase in cellular lipid content.

FIG. 16A shows a protocol for a lipid accumulation test where lipids were cultured with cells for 12 hours.

FIG. 16B shows the effect of an LNA gapmer antisense oligonucleotide (ASO) targeting Cyclin D2 in human induced-pluripotent stem cell derived cardiomyocytes (hiPSC-CMs) using BODIPY-Palmitate (BODIPY C16) (i.e., a fluorescently (488) tagged fatty acid). FIG. 16B shows images for four groups: Scramble ASO, ANGII+Scramble ASO, ANGII+Cyclin D2 ASO (SEQ ID NO: 5), ANGII+Cyclin D2 ASO (SEQ ID NO: 7).

FIG. 16C shows the effect of an LNA gapmer antisense oligonucleotide (ASO) targeting Cyclin D2 in human induced-pluripotent stem cell derived cardiomyocytes (hiPSC-CMs) (acquired from Cellular Dynamics International (aged to day 30)) on accumulation of fatty acids (mean (solid line), n=3, >50 cardiomyocytes measured per n) for four groups: Scramble ASO, ANGII+Scramble ASO, ANGII+Cyclin D2 ASO (SEQ ID NO: 5), ANGII+Cyclin D2 ASO (SEQ ID NO: 7). The box and violin plot generated by an unpaired t test, shows Cyclin D2 ASOs significantly (p<0.05) suppresses ANGII-induced increase in cellular lipid content.

FIG. 17A shows a protocol for examining the effect of high concentrations of Fatty Acids in the culture media on cardiomyocyte viability.

FIG. 17B shows the effect of an LNA gapmer antisense oligonucleotide (ASO) targeting Cyclin D2 in human induced-pluripotent stem cell derived cardiomyocytes (AICS-0060-027 cell line differentiated and aged to day 30) on viability in response to high concentration of fatty acid (palmitate, 800 micromolar) in the culture medium in the presence of ANGII (mean with SEM, n=5, >50 cardiomyocytes measured per n) for two groups: ANGII+Scramble ASO, ANGII+Cyclin D2 ASO (SEQ ID NO: 5).

### 6. DETAILED DESCRIPTION

As provided herein it has been demonstrated that reducing or inhibiting Cyclin D2 expression (i) suppresses cardiomyocyte hypertrophy, (ii) promotes FAO, and (iii) reduces fatty acid accumulation and fatty acid-induced lipotoxocity in culture conditions using primary mammalian cardiomyocytes derived from rats and/or human cardiomyocytes derived from induced pluripotent stem cells. Given that both hypertrophy, metabolic remodeling, and lipotoxicity are observed during induced heart failure in mice and rats and heart failure in humans, indicates that inhibiting Cyclin D2 expression (e.g. through siRNAs or ASOs targeting Cyclin D2 mRNA) can be used to treat heart failure.

In the adult heart, in response to various pathologic and growth stimuli, cardiomyocytes rarely exhibit signs of cell cycle progression (e.g., S-phase (i.e., DNA synthesis activity) or mitosis) (Zebrowski, D. C., Rev. Physiol. Biochem. Pharmacol., 165: p. 67-96 (2013)). However, in response to pathologic stress, postnatal mammalian cardiomyocytes nevertheless appear to re-enter the G1 phase of the Cell Cycle (i.e., they transition from G0 to G1) (Zebrowski, D. C., Rev. Physiol. Biochem. Pharmacol., 165: p. 67-96 (2013)). Notably, Cyclin D2 plays a role in entry into G1 of the cell cycle. As such, Cyclin D2 is modulated during pathologic stimuli in postnatal mammalian cardiomyocytes, and its modulation may be required for the development of pathologic hypertrophy. However, to date, the requirement for D-type Cyclins with regards to regulation of hypertrophy requires further experimentation.

Pathologic stimuli that result in heart failure also result in postnatal cardiomyocytes reverting to a fetal metabolic profile (i.e., whereby their primary source of energy production is Glycolysis not FAO)—also known as metabolic remodeling (Barger, P. M., Am. J. Med. Sci., 318(1): p. 36-42 (1999); Allard, M. F., et al., Am. J. Physiol., 267(2 Pt 2): p. H742-50 (1994)). During fetal heart development, when cardiomyocytes are highly proliferative, the primary source of energy production for cardiomyocytes is glycolysis with glucose as the primary substrate. During neonatal development, when cardiomyocytes exit the cell cycle and become terminally differentiated, cardiomyocytes undergo a metabolic shift whereby the primary source of energy production transitions to Fatty Acid Oxidation (FAO) with free fatty acids and triacylglycerol (TAG) as the primary substrates. Consistent with this, genes required for FAO (e.g., MCAD and mCPT-1) are upregulated as maturation progresses (Kelly, D. P., et al., J. Biol. Chem, 264(32): p. 18921-5 (1989); Onay-Besikci, A., et al., Am. J. Physiol. Heart Circ. Physiol., 284(1): p. H283-9 (2003). The metabolic switch is completed in the adult heart whereby 95% of ATP generated in the heart is due to either Glucose, or Fatty Acid, Oxidation (40-70% of which is FAO) with only 5% of ATP generated by Glycolysis (Lopaschuk, G. D, J. Cardiovasc. Pharmacol., 56(2): p. 130-40 (2010); Gibb, A. A. and B. G. Hill, Circ. Res., 2018. 123(1): p. 107-128 (2018); Lopaschuk, G. D., et al., Physiol Rev., 2010. 90(1): p. 207-58 (2010)). During pathologic stress, cardiomyocytes revert to a fetal-like metabolic state whereby they are less able to utilize Fatty Acids as an energy source. The mechanisms that regulate the metabolic switch away from FAO are not fully understood. To date, the role of Cyclin D2 has yet to be investigated with regards to regulation of metabolism in cardiomyocytes, particularly with regards to the reduced ability to perform FAO observed under conditions of chronic pathologic cardiac stress.

Without wishing to be bound by theory, there is increasing evidence that FA-uptake from the extracellular environment, under conditions of suppressed FAO, can result in lipotoxicity (i.e. cell death due to accumulation of excess lipids). Lipid overload in the cell has been reported to involve endoplasmic reticulum stress, alterations in autophagy, de novo ceramide synthesis, and oxidative stress. Furthermore, lipotoxicity has been implicated as a contributing factor in cardiac dysfunction and the development of heart failure in animal models of heart failure and in humans with heart failure (Abdurrachim D, et. al., Cardiovascular Research (2015) 106, 194-205; Konstantinos Drosatos and P. Christian Schulze, Curr. Heart Fail Rep, 2013 (June; 10(2): 109-121; Leggat et al, Clinical Science, Oct. 13, 2021). However, the mechanisms that regulate the fatty acid accumulation and contribute to lipotoxicity in cardiomyocytes are not fully understood. To date, the role of Cyclin D2 has yet to be investigated with regards to regulation of lipotoxicity in cardiomyocytes, particularly with regards to the lipid accumulation observed under conditions of chronic pathologic cardiac stress.

As such, in one aspect, the present disclosure relates to methods of treating heart failure (e.g., reducing or inhibiting cardiac hypertrophy or by increasing utilization of fatty acid oxidation or by inhibit fatty acid build-up and fatty acid-induced lipotoxicity) where the method includes administering to the patient a therapeutically effective amount of an oligonucleotide-based therapeutic comprising a polynucleotide sequence that is at least partially complementary to a nucleic acid sequence with a Cyclin D2 (CCND2) transcript such to suppress transcript levels of Cyclin D2. For example, Applicant showed that reducing or inhibiting Cyclin D2 expression in cardiomyocytes using an oligonucleotide-based therapeutic (e.g., an ASO or a siRINA targeting Cyclin D2) (i) suppressed pathologic hypertrophic growth, (ii) preserved/increased FAO capacity, and (iii) reduced of fatty acid accumulation and reduced lipotoxicity.

### 6.1. Definitions

As used herein, the term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, for example, 10 percent, up or down (higher or lower).

As used herein, a “coding region” or “coding sequence” is a portion of polynucleotide which consists of codons translatable into amino acids. Although a “stop codon” (TAG, TGA, or TAA) is typically not translated into an amino acid, it can be considered to be part of a coding region, but any flanking sequences, for example promoters, ribosome binding sites, transcriptional terminators, introns, untranslated regions (“UTRs”), and the like, are not part of a coding region. The boundaries of a coding region are typically determined by a start codon at the 5′ terminus, encoding the amino terminus of the resultant polypeptide, and a translation stop codon at the 3′ terminus, encoding the carboxyl terminus of the resulting polypeptide.

As used herein, the term “non-coding region” as used herein means a nucleotide sequence that is not a coding region. Examples of non-coding regions include, but are not limited to, promoters, ribosome binding sites, transcriptional terminators, introns, untranslated regions (“UTRs”), non-coding exons and the like. Some of the exons can be wholly or part of the 5′ untranslated region (5′ UTR) or the 3′ untranslated region (3′ UTR) of each transcript. The untranslated regions are important for efficient translation of the transcript and for controlling the rate of translation and half-life of the transcript.

As used herein, the term “region” when used in the context of a nucleotide sequence refers to a section of that sequence. For example, the phrase “region within a nucleotide sequence” refers to a sequence shorter than the nucleotide sequence, but longer than at least 10 nucleotides located within the particular nucleotide sequence or the complement of the nucleotides sequence, respectively.

As used herein, term “downstream,” when referring to a nucleotide sequence, means that a nucleic acid or a nucleotide sequence is located 3′ to a reference nucleotide sequence. In certain embodiments, downstream nucleotide sequences relate to sequences that follow the starting point of transcription. For example, the translation initiation codon of a gene is located downstream of the start site of transcription.

As used herein, the term “upstream” refers to a nucleotide sequence that is located 5′ to a reference nucleotide sequence.

As used herein, the term “conjugate” refers to an oligonucleotide-based therapeutic covalently linked to a non-nucleotide moiety (e.g., a conjugate moiety).

As used herein, the term “activated oligonucleotide-based therapeutic” refers to an oligonucleotide-based therapeutic that is covalently linked to a functional moiety that permits covalent linkage of the oligonucleotide-based therapeutic to one or more conjugated moieties. Typically, a functional moiety will comprise a chemical group that is capable of covalently bonding to the oligonucleotide-based therapeutic via, e.g., a 3′-hydroxyl group or the exocyclic NH2 group of the adenine base, a spacer that can be hydrophilic and a terminal group that is capable of binding to a conjugated moiety (e.g., an amino, sulfhydryl or hydroxyl group). In some embodiments, this terminal group is not protected, for example, is an NH2 group.

As used herein, the term “complementarity” refers to determining the degree of “complementarity” between the oligonucleotide-based therapeutics provided herein (or a region thereof) and the target region of the nucleic acid which encodes mammalian Cyclin D2 (e.g., the Cyclin D2 gene), such as those disclosed herein, the degree of “complementarity” (also, “homology” or “identity”) is expressed as the percentage identity (or percentage homology) between the sequence of the oligonucleotide-based therapeutic (or region thereof) and the sequence of the target region (or the reverse complement of the target region) that best aligns therewith. The percentage is calculated by counting the number of aligned bases that are identical between the two sequences, dividing by the total number of contiguous monomers in the oligonucleotide-based therapeutics, and multiplying by 100. In such a comparison, if gaps exist, it is preferable that such gaps are merely mismatches rather than areas where the number of monomers within the gap differs between the oligonucleotide-based therapeutic of the disclosure and the target region.

As used herein, term “complement” indicates a sequence that is complementary to a reference sequence. It is well known that complementarity is the base principle of DNA replication and transcription as it is a property shared between two DNA or RNA sequences, such that when they are aligned antiparallel to each other, the nucleotide bases at each position in the sequences will be complementary. For example, the complement of a sequence of 5′ “ATGC” 3′ can be written as 3′ “TACG” 5′ or 5′ “GCAT” 3′3. As used herein, the terms “reverse complement”, “reverse complementary”, and “reverse complementarity” are interchangeable with the terms “complement”, “complementary”, and “complementarity.” In some embodiments, the term “complementary” refers to 100% match or complementarity (i.e., fully complementary) to a contiguous nucleic acid sequence within a Cyclin D2 transcript. In some embodiments, the term “complementary” refers to at least about 80%, at least about 85%, at least about 90%, at least about 91%, at least about 92%, at least about 93%, at least about 94%, at least about 95%, at least about 96%, at least about 97%, at least about 98%, or at least about 99% match or complementarity to a contiguous nucleic acid sequence within a Cyclin D2 transcript.

As used herein, the term “effective amount” of an oligonucleotide-based therapeutic is an amount sufficient to carry out a specifically stated purpose. An “effective amount” can be determined empirically and in a routine manner, in relation to the stated purpose.

As used herein, term “expression” refers to a process by which a polynucleotide produces a gene product, for example, a RNA or a polypeptide. It includes, without limitation, transcription of the polynucleotide into messenger RNA (mRNA) and the translation of an mRNA into a polypeptide. Expression produces a “gene product.” As used herein, a gene product can be either a nucleic acid, for example, a messenger RNA produced by transcription of a gene, or a polypeptide which is translated from a transcript. Gene products described herein further include nucleic acids with post transcriptional modifications, for example, polyadenylation or splicing, or polypeptides with post translational modifications, for example, methylation, glycosylation, the addition of lipids, association with other protein subunits, or proteolytic cleavage.

As used herein, the term “inhibiting” refers to the oligonucleotide-based therapeutic reducing the expression of the CCND2 gene transcript and/or CCND2 protein in a cardiomyocyte or a tissue (e.g., heart). In some embodiments, the term “inhibiting” refers to complete inhibition (100% inhibition or non-detectable level) of Cyclin D2 mRNA transcript or CCND2 protein. In other embodiments, the term “inhibiting” refers to at least 5%, at least 10%, at least 15%, at least 20%, at least 25%, at least 30%, at least 35%, at least 40%, at least 45%, at least 50%, at least 60%, at least 70%, at least 80%, at least 90%, at least 95% or at least 99% inhibition of Cyclin D2 gene transcript and/or CCND2 protein expression in a cell or a tissue.

As used herein, the term “interfering activity” refers to an oligonucleotide-based therapeutic binding to and reducing or inhibiting the RNA or protein of a target gene.

As used herein, the term “naturally occurring variant thereof” refers to variants of the CCND2 polypeptide sequence or CCND2 nucleic acid sequence (e.g., mRNA transcript) which exist naturally within the defined taxonomic group, such as mammalian, such as mouse, monkey, and human. Typically, when referring to “naturally occurring variants” of a polynucleotide the term also can encompass any allelic variant of the CCND2-encoding genomic DNA which is found at Chromosome 12 (i.e., residues 4273762 to 4305353of GenBank Accession No. NC_000012.12) by chromosomal translocation or duplication, and the RNA, such as mRNA derived therefrom. “Naturally occurring variants” can also include variants derived from alternative splicing of the CCND2 mRNA. When referenced to a specific polypeptide sequence, e.g., the term also includes naturally occurring forms of the protein, which can therefore be processed, e.g., by co-translational or post-translational modifications, such as signal peptide cleavage, proteolytic cleavage, glycosylation, among other post-translational modifications.

As used herein, the term “nucleic acids,” “nucleotides” or “polynucleotides” is intended to encompass plural nucleic acids. In some embodiments, the term “nucleic acids,” “nucleotides” or “polynucleotides” refers to a target sequence, for example, a pre-mRNA, mRNAs, or DNAs in vivo or in vitro. When the term refers to the nucleic acids, nucleotides, or polynucleotides in a target sequence, the nucleic acids or nucleotides can be naturally occurring sequences within a cell (e.g., a cardiomyocyte). In other embodiments, nucleic acids,” “nucleotides” or “polynucleotides” refer to a sequence in the oligonucleotide-based therapeutic of the present disclosure. When the term refers to a sequence in the oligonucleotide-based therapeutic, the nucleic acids, nucleotides, or polynucleotides are not naturally occurring (e.g., the nucleic acids, nucleotides, or polynucleotides are chemically synthesized, enzymatically produced, recombinantly produced, or any combination thereof). In one embodiment, the nucleic acids or nucleotides in the oligonucleotide-based therapeutic are produced synthetically or recombinantly but are not a naturally occurring sequence or a fragment thereof. In another embodiment, the nucleic acids or nucleotides in the oligonucleotide-based therapeutic are not naturally occurring because they contain at least one nucleotide analog that is not naturally occurring in nature (e.g., a modified nucleotide).

As used herein, the term “nucleotide” refers to a glycoside comprising a sugar moiety, a base moiety and a covalently linked group (linkage group), such as a phosphate or phosphorothioate internucleotide linkage group, and covers both naturally occurring nucleotides, such as DNA or RNA, and non-naturally occurring nucleotides comprising modified sugar and/or base moieties, which are also referred to as “nucleotide analogs” herein. Herein, a single nucleotide (unit) can also be referred to as a monomer or nucleic acid unit. In certain embodiments, the term “nucleotide analogs” refers to nucleotides having modified sugar moieties.

As used herein, the term “nucleoside” refers to a glycoside comprising a sugar moiety and a base moiety and can therefore be used when referring to the nucleotide units, which are covalently linked by the internucleotide linkages between the nucleotides of the ASO. In the field of biotechnology, the term “nucleotide” is often used to refer to a nucleic acid monomer or unit. In the context of an oligonucleotide-based therapeutic (e.g., an ASO), the term “nucleotide” can refer to the base alone, i.e., a nucleobase sequence comprising cytosine (DNA and RNA), guanine (DNA and RNA), adenine (DNA and RNA), thymine (DNA) and uracil (RNA), in which the presence of the sugar backbone and internucleotide linkages are implicit. Likewise, particularly in the case of oligonucleotides where one or more of the internucleotide linkage groups are modified, the term “nucleotide” can refer to a “nucleoside.” For example, the term “nucleotide” can be used, even when specifying the presence or nature of the linkages between the nucleosides.

As used herein, the term “oligonucleotide” or “polynucleotide” can be used interchangeably, and refers to polymers of nucleotides, and includes, but is not limited to, single-stranded or double-stranded nucleic acid molecules of DNA, RNA, or DNA/RNA hybrid, oligonucleotide strands containing regularly and irregularly alternating deoxyribosyl portions and ribosyl portions, as well as modified and naturally or unnaturally existing frameworks for such oligonucleotides.

As used herein, the term “transcript” refers to a primary transcript that is synthesized by transcription of DNA and becomes a messenger RNA (mRNA) after processing, for example, a precursor messenger RNA (pre-mRNA), and the processed mRNA itself. The term “transcript” can be interchangeably used with “pre-mRNA” and “mRNA.” After DNA strands are transcribed to primary transcripts, the newly synthesized primary transcripts are modified in several ways to be converted to their mature, functional forms to produce different proteins and RNAs such as mRNA, tRNA, rRNA, lncRNA, miRNA and others. Thus, the term “transcript” can include exons, introns, 5′ UTRs, and 3′ UTRs.

As used herein, the term “pathological stimuli” refers to intrinsic and extrinsic stimuli that results in heart failure. Heart failure is identified, at least in part, by cardiac hypertrophy, metabolic remodeling, among other characteristics.

As used herein, the terms “treating” or “treatment” or “to treat” refer to both therapeutic measures that cure, slow down, lessen symptoms of, and/or halt progression of a diagnosed pathologic condition or disorder and prophylactic or preventative measures that prevent and/or slow the development of a targeted pathologic condition or disorder. Thus, those in need of treatment include those already with the disorder; those prone to have the disorder; and those in whom the disorder is to be prevented. In certain embodiments, a subject is successfully “treated” for a disease or condition disclosed elsewhere herein according to the methods provided herein if the patient shows, for example, total, partial, or transient alleviation or elimination of symptoms associated with the disease or disorder.

Throughout this specification and claims, “composition” and “pharmaceutical composition” are used interchangeably.

Throughout this specification and claims, the word “comprise” or variations thereof, such as “comprises” or “comprising,” will be understood to imply the inclusion of a stated integer or group of integers but not the exclusion of any other integer or group of integers.

Before the present invention is further described, it is to be understood that this invention is not limited to particular embodiments described, as such embodiments may vary. It is also to be understood that the terminology used herein is for the purpose of describing particular embodiments only, and is not intended to be limiting, since the scope of the presently claimed invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the presently claimed invention. The upper and lower limits of these smaller ranges may independently be included in the smaller ranges, and are also encompassed within the claimed invention, subject to any specifically excluded limit in the stated range. Where the stated range includes one or both of the limits, ranges excluding either or both of those included limits are also included in the claimed invention.

Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

It must be noted that as used herein and in the appended claims, the singular forms “a,” “an,” and “the” include plural referents unless the context clearly dictates otherwise. Thus, for example, reference to “an oligonucleotide-based therapeutic” includes a plurality of such cardiomyocytes and reference to “the oligonucleotide-based therapeutic” includes reference to one or more oligonucleotide-based therapeutics and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

It is appreciated that certain features of the disclosure, which are, for clarity, described in the context of separate embodiments, may also be provided in combination in a single embodiment. Conversely, various features of the present disclosure, which are, for brevity, described in the context of a single embodiment, may also be provided separately or in any suitable sub-combination. All combinations of the embodiments pertaining to the invention are specifically embraced by the presently claimed invention and are disclosed herein just as if each and every combination was individually and explicitly disclosed. In addition, all sub-combinations of the various embodiments and elements thereof are also specifically embraced by the presently claimed invention and are disclosed herein just as if each and every such sub-combination was individually and explicitly disclosed herein.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

### 6.2. Other Interpretational Conventions

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

### 6.3. Methods of Treatment

In a first aspect, the present disclosure provides methods for treating a patient having, or at risk of having, heart failure, comprising: administering to the patient a therapeutically effective amount of an oligonucleotide-based therapeutic comprising a polynucleotide sequence that is at least partially complementary to a nucleic acid sequence with a Cyclin D2 (CCND2) transcript, wherein Cyclin D2 expression in the patient is inhibited or reduced after the administration, thereby treating the patient having, or at risk of having, heart failure. In such cases, reducing or inhibiting Cyclin D2 expression treats the heart failure, or symptoms thereof.

In another aspect, the present disclosure provides methods for reducing or preventing hypertrophy of a plurality of cardiomyocytes, comprising: contacting the cardiomyocytes with an effective amount of an oligonucleotide-based therapeutic comprising a polynucleotide sequence that is at least partially complementary to a nucleic acid sequence with a Cyclin D2 (CCND2) transcript, wherein Cyclin D2 expression in the cardiomyocytes is inhibited or reduced after contacting the cardiomyocytes, thereby reducing or preventing hypertrophy of a plurality of cardiomyocytes. In some embodiments, the patient has, or is at risk of having, heart failure, where the heart failure comprises cardiac hypertrophy. In such cases, reducing or inhibiting of Cyclin D2 expression reduces or prevents hypertrophy of the plurality of cardiomyocytes. In some embodiments, the plurality of cardiomyocytes is that of the heart of a patient.

In some embodiments, reducing hypertrophy is determined by measuring cardiomyocyte area of individual cardiomyocytes. In some embodiments, reducing hypertrophy is determined by measuring mean cardiomyocyte area of a population cardiomyocytes. For example, a reduction in mean cardiomyocyte area as compared to a control (e.g., mean cardiomyocyte area of cardiomyocytes exposed to pathological stimuli but not treated with the oligonucleotide-based therapeutics provided herein) indicates reduction of hypertrophy.

In some embodiments, the methods provided herein reduce mean cardiomyocyte area by at least 10% (e.g., at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%) as compared to a control (e.g., mean cardiomyocyte area of cardiomyocytes exposed to pathological stimuli but not treated with the oligonucleotide-based therapeutics provided herein).

In some embodiments, preventing hypertrophy is determined by measuring cardiomyocyte area of individual cardiomyocytes. In some embodiments, preventing hypertrophy is determined by measuring mean cardiomyocyte area of a population of cardiomyocytes. For example, mean cardiomyocyte area measured for a sample treated with the oligonucleotide-based therapeutics provided herein that is similar (e.g., not statistically significantly different) to the mean cardiomyocyte area as measured for a control indicates prevention of hypertrophy.

In another aspect, the present disclosure provides methods for increasing utilization of fatty-acid oxidation in a cardiomyocyte, comprising: contacting the cardiomyocyte with an effective amount of an oligonucleotide-based therapeutic comprising a polynucleotide sequence that is at least partially complementary to a nucleic acid sequence with a Cyclin D2 (CCND2) transcript, wherein Cyclin D2 expression is inhibited or reduced after contacting the cardiomyocyte, thereby increasing utilization of fatty acid oxidation. In such cases, reducing or inhibiting Cyclin D2 expression increases the cardiomyocyte's utilization of fatty-acid oxidation.

In some embodiments, the present disclosure provides methods for maintaining utilization of fatty-acid oxidation in a cardiomyocyte, comprising: contacting the cardiomyocyte with an effective amount of an oligonucleotide-based therapeutic comprising a polynucleotide sequence that is at least partially complementary to a nucleic acid sequence with a Cyclin D2 (CCND2) transcript, wherein Cyclin D2 expression is inhibited or reduced after contacting the cardiomyocyte. For example, contacting the cardiomyocyte with an effective amount of an oligonucleotide-based therapeutic enables the cardiomyocyte to maintain fatty-acid oxidation as a source of energy as compared to a control (e.g., amount of energy generated using FAO in a cardiomyocyte exposed to pathological stimuli but not treated with the oligonucleotide-based therapeutics provided herein). In some cases, as a result of the methods provided herein, a cardiomyocyte generates between about 40% to about 70% of its energy from fatty acid oxidation, which is similar to levels in the adult heart. In some cases, as a result of the methods provided herein, a cardiomyocyte generates between about 45% to about 65% or about 50% to about 60% of its energy from fatty acid oxidation. In some cases, as a result of the methods provided herein, a cardiomyocyte generates between about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, or about 70% or more of its energy from fatty acid oxidation.

In some embodiments, the methods provided herein increase the amount of energy generated using fatty acid oxidation in a cardiomyocyte by least 10% (e.g., at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, or 200% or more) as compared to a control (e.g., amount of energy generated using FAO in a cardiomyocyte exposed to pathological stimuli but not treated with the oligonucleotide-based therapeutics provided herein).

In some embodiments, increases in cardiomyocyte utilization of fatty-acid oxidation is determined by measuring one or more of: expression of a biomarker for FAO (e.g., MCPT1 or MCAD), spare respiratory capacity (SRC), or oxygen consumption rate (OCR), or suppressing the accumulation of FAO in the cytosol of the cardiomyocyte when cultured with fatty acids or any combination thereof.

In some embodiments, increases in cardiomyocyte utilization of fatty-acid oxidation is determined by measuring expression of a biomarker for FAO (e.g., MCPT1 or MCAD). For example, an increase in expression of a biomarker for FAO (e.g., MCPTA or MCA) as compared to a control (e.g., expression levels of a biomarker for FAO in a cardiomyocyte exposed to pathological stimuli but not treated with the oligonucleotide-based therapeutics provided herein) indicates increase utilization of FAO in the cardiomyocyte.

In some embodiments, increases in cardiomyocyte utilization of fatty-acid oxidation is determined by measuring oxygen consumption rate (OCR) of a cardiomyocyte (e.g., a plurality of cardiomyocytes). For example, an increase in OCR of a cardiomyocyte as compared to a control (e.g., OCR in a cardiomyocyte exposed to pathological stimuli but not treated with the oligonucleotide-based therapeutics provided herein) indicates increase utilization of FAO in the cardiomyocyte(s).

In some embodiments, increases in cardiomyocyte utilization of fatty-acid oxidation is determined by measuring spare respiratory capacity (SRC) of a cardiomyocyte (e.g., a plurality of cardiomyocytes). For example, an increase in SRC of a cardiomyocyte as compared to a control (e.g., SRC in a cardiomyocyte exposed to pathological stimuli but not treated with the oligonucleotide-based therapeutics provided herein) indicates increase utilization of FAO in the cardiomyocyte(s).

In some embodiments, increases in cardiomyocyte utilization of fatty-acid oxidation is determined measuring the accumulation of FAO in the cytosol of the cardiomyocyte when cultured with fatty acids. For example, a decrease (or reduction) in the accumulation of FAO in the cytosol of the cardiomyocyte when cultured with fatty acids as compared to a control (e.g. accumulation of FAO in the cytosol of the cardiomyocyte exposed to pathological stimuli but not treated with the oligonucleotide-based therapeutics provided herein) indicates a decreases (or reduction) of the accumulation of FAO in the cytosol in the cardiomyocyte(s).

In some embodiments, maintenance of fatty-acid oxidation as a source of energy production in a cardiomyocyte (e.g., a plurality of cardiomyocytes) is determined by measuring one or more of: expression of a biomarker for FAO (e.g., MCPT1 or MCAD), spare respiratory capacity (SRC), or oxygen consumption rate (OCR).

In some embodiments, maintenance of fatty-acid oxidation as a source of energy production in a cardiomyocyte (e.g., a plurality of cardiomyocytes) is determined by measuring expression of a biomarker for FAO (e.g., MCPT1 or MCAD). For example, expression of a biomarker for FAO (e.g., MCPTA or MCAD) that is similar (e.g., not statistically significantly different) to a control (e.g., expression levels of a biomarker for FAO in a cardiomyocyte exposed to pathological stimuli but not treated with the oligonucleotide-based therapeutics provided herein) indicates maintenance of FAO as a source of energy production in a cardiomyocyte.

In some embodiments, maintenance of fatty acid oxidation as a source of energy production in cardiomyocytes is determined by measuring oxygen consumption rate (OCR) of a cardiomyocyte (e.g., a plurality of cardiomyocytes). For example, an OCR of a cardiomyocyte that is similar (e.g., not statistically significantly different) to a control (e.g., OCR in a cardiomyocyte exposed to pathological stimuli but not treated with the oligonucleotide-based therapeutics provided herein) indicates maintenance of FAO as a source of energy production in a cardiomyocyte.

In some embodiments, maintenance of fatty acid oxidation as a source of energy production in cardiomyocytes is determined by measuring maximal respiration and spare respiratory capacity (SRC) of a cardiomyocyte (e.g., a plurality of cardiomyocytes). For example, an SRC of a cardiomyocyte that is similar (e.g., not statistically significantly different) to a control (e.g., SRC in a cardiomyocyte exposed to pathological stimuli but not treated with the oligonucleotide-based therapeutics provided herein) indicates maintenance of FAO as a source of energy production in a cardiomyocyte.

In another aspect, the present disclosure provides methods for reducing or preventing fatty acid (lipid) accumulation and fatty acid-induced lipotoxicity in a cardiomyocyte. The method comprises contacting a cardiomyocyte with an effective amount of an oligonucleotide-based therapeutic comprising a polynucleotide sequence that is at least partially complementary to a nucleic acid sequence within a Cyclin D2 (CCND2) transcript, wherein Cyclin D2 expression is inhibited or reduced after contacting the cardiomyocyte with the oligonucleotide-based therapeutic, thereby reducing fatty acid (lipid) accumulation and fatty-acid induced lipotoxicity. In some embodiments, reducing or inhibiting Cyclin D2 expression reduces lipid accumulation and lipotoxicity in the cardiomyocyte.

In some embodiments, the patient has, or is at risk of having, lipotoxicity, where the lipotoxicity induced by fatty acid accumulation. In such cases, reducing or inhibiting of Cyclin D2 expression reduces or prevents lipid accumulation in the cardiomyocyte.

In some embodiments, reducing or preventing lipid accumulation and/or lipotoxicity is determined by measuring lipid content in the cell (or the cell membrane). A non-limiting method for determining levels of lipotoxicity include measuring uptake of 7-AAD. Uptake of 7-AAD reflects the loss of cytosolic membrane integrity and this is a proxy for cell death.

In some embodiments, the methods provided herein reduce lipotoxicity (e.g., as measured by 7-AAD uptake) by at least 10% (e.g., at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%) as compared to a control (e.g., 7-ADD uptake in a cardiomyocyte exposed to pathological stimuli but not treated with the oligonucleotide-based therapeutics provided herein).

In some embodiments, the methods provided herein reduce lipotoxicity (e.g., as measured by 7-AAD uptake) by at least 1.5-fold (at least 2-fold, at least 3-fold, at least 4-fold, at least 5-fold, at least 6-fold, at least 7-fold, at least 8-fold, at least 9-fold, or at least 10-fold) as compared to a control (e.g., 7-AAD uptake in a cardiomyocyte exposed to pathological stimuli but not treated with the oligonucleotide-based therapeutics provided herein).

In another aspect, the present disclosure provides methods for inhibiting or reducing Cyclin D2 expression in a cell, comprising: contacting the cell with an effective amount of an oligonucleotide-based therapeutic comprising a polynucleotide sequence that is at least partially complementary to a nucleic acid sequence with a Cyclin D2 (CCND2) transcript, wherein Cyclin D2 expression is inhibited or reduced after contacting the cell. In some embodiments, the cell is a cardiomyocyte.

Non-limiting examples of measuring Cyclin D2 mRNA or protein levels include RT-PCR, qRT-PCR, ddRT-PCR, RNA-sequencing, Northern blot, Western blot. ELISA, immunofluorescence, immunohistochemistry, and mass spectrometry, among other methods known in the art or provided herein.

In some embodiments, the methods provided herein result in a reduction in Cyclin D2 mRNA expression levels by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more compared to a control cardiomyocyte not contacted with any of the oligonucleotide-based therapeutics described herein.

In some embodiments, the methods provided herein result in a reduction in Cyclin D2 protein levels by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more compared to a control cardiomyocyte not contacted with any of the oligonucleotide-based therapeutics described herein.

In some embodiments, patients administered the oligonucleotide-based therapeutic are patients having an increased risk of heart failure, patients suffering from heart failure (e.g., congestive heart failure), post-myocardial infarction patients or patients with congenital heart diseases associated to cardiac hypertrophy, such as pulmonal vein stenosis, atrial or ventricular septum defects, patients suffering from genetic conditions leading to heart failure (e.g., hypertrophic cardiomyopathy).

### 6.4. Oligonucleotide-Based Therapeutic

In one aspect, the present disclosure provides oligonucleotide-based therapeutics for modulating expression of Cyclin D2. The oligonucleotide-based therapeutics for modulating expression of Cyclin D2 includes a polynucleotide sequence that is at least partially complementary to a nucleic acid sequence within a Cyclin D2 (CCND2) transcript. In some embodiments, the polynucleotide sequence of oligonucleotide-based therapeutics comprises a sequence having at least about 80%, at least about 85%, at least about 90%, at least about 95%, or about 100% complementarity to the nucleic acid sequence within the Cyclin D2 transcript. In some embodiments, the Cyclin D2 transcript is selected from a sequence of SEQ ID NO: 1-3.

In some embodiments, the oligonucleotide-based therapeutic includes a pool of at least two (e.g., at least three, at least four, at least five, at least six, at least seven, at least eight, at least nine, or ten or more) oligonucleotide-based therapeutics. For example, the methods provided herein include administering (or contacting) a cardiomyocyte with a pool of oligonucleotide-based therapeutics where each of the individual oligonucleotide-based therapeutics comprises a different polynucleotide sequence that is at least partially complementary to a nucleic acid sequence with a Cyclin D2 (CCND2) transcript. In such cases, each oligonucleotide-based therapeutic inhibits or reducing Cyclin D2 expression either independently or in a synergistic manner.

In some embodiments, administering (or contacting a cell with) the oligonucleotide-based therapeutics for modulating expression of Cyclin D2 results in reduction in Cyclin D2 expression (protein/transcript) by at least at least 5%, at least 10%, at least 15%, at least 20%, at least 25%, at least 30%, at least 35%, at least 40%, at least 45%, at least 50%, at least 60%, at least 70%, at least 80%, at least 90%, at least 95% or at least 99%) as compared to Cyclin D2 expression (protein/transcript) in a patient not treated or cell not contacted with the oligonucleotide-based inhibitor.

Non-limiting examples of oligonucleotide-based therapeutic include: anti-sense oligonucleotides (ASO), short interfering RNA (siRNA), single-stranded siRNAs (ssRNAs), small temporal RNA (stRNA), short hairpin RNA (shRNA), locked nucleic acids (LNA), antagomirs aptamer, peptide nucleic acids (PNA), and micro RNA (miRNA).

### 6.4.1. Cyclin D2

This disclosure features oligonucleotide-based therapeutics capable of modulating expression of Cyclin D2. As used herein, Cyclin D2 (refers to the Ccnd2 gene or CCND2 polypeptide). In some embodiments, modulating expression of Cyclin D2 includes reducing or inhibiting expression of Cyclin D2 mRNA or protein. In some embodiments, the oligonucleotide-based therapeutic can affect indirect inhibition of Cyclin D2 protein through the reduction in Cyclin D2 mRNA levels in a mammalian cell (e.g., a cardiomyocyte). In particular, the present disclosure is directed to oligonucleotide-based therapeutics that target one or more regions of the Cyclin D2 pre-mRNA (e.g., intron regions, exon regions, and/or exon-intron junction regions). As used herein, the terms “Cyclin D2” or “CCND2” can refer to Cyclin D2 from one or more species (e.g., humans, non-human primates, dogs, cats, guinea pigs, rabbits, rats, mice, horses, and cattle).

Cyclin D2 belongs to the highly conserved cyclin family. Cyclins function as regulators of CDK kinases during the cell cycle. Different members of the cyclin family exhibit distinct expression and degradation patterns which contribute to the temporal coordination of each mitotic event. Cyclin D2 forms a complex with CDK4 or CDK6 and functions as a regulatory subunit of the complex, whose activity is required for cell cycle GUS transition. In some cases, Cyclin D2 has been shown to interact with and be involved in the phosphorylation of tumor suppressor protein Rb. However, previous work did not establish the functional consequences of this regulation. As provided herein, this disclosure illustrates that reducing or inhibiting Cyclin D2 using an oligonucleotide-based therapeutic reduces pathological hypertrophy, preserves Fatty Acid Oxidation capacity, and preserves cardioprotective hormones in cardiomyocytes contacted with the therapeutic.

In some embodiments, the nucleic acid sequence for the Cyclin D2 gene can be found at GenBank Accession Number NC_000012.12. In some embodiments, the Cyclin D2 pre-mRNA transcript (e.g., the sequence corresponding to NM_001759.4) has a sequence of SEQ ID NO: 1, or a naturally occurring variant thereof. In some embodiments, the Cyclin D2 mRNA transcript (e.g., the sequence corresponding to nucleotides 279 to 1148 of SEQ ID NO: 1) has a sequence of SEQ ID NO: 2. In some embodiments, the Cyclin D2 mRNA transcript has the sequence of SEQ ID NO: 3, which is the sequence provided in SEQ ID NO: 2 except nucleotides “T” is shown as “U” in the mRNA. In some embodiments, Cyclin D2 protein has the amino acid sequence of SEQ ID NO: 4. In some embodiments, a Cyclin D2 protein includes the SEQ ID NO: 4 having one or more (e.g., one, two, three, four, five, six, seven, eight, nine, ten, or more amino acid substitutions, deletions or insertions as compared to a wild type Cyclin D2 protein)).

In some embodiments, an oligonucleotide based therapeutic comprises a polynucleotide sequence that is at least partially complementary to a nucleic acid sequence within a Cyclin D2 (CCND2) transcript, or a naturally occurring variants thereof of such nucleic acid molecules encoding mammalian CCND2.

In some embodiments, the “nucleic acid sequence within a Cyclin D2 (CCND2) transcript” comprises an intron of a Cyclin D2 protein-encoding nucleic acid sequence, or naturally occurring variants thereof, and RNA nucleic acids derived therefrom, e.g., pre-mRNA. In some embodiments, the target nucleic acid comprises an exon region of a Cyclin D2 protein-encoding nucleic acids, or naturally occurring variants thereof, and RNA nucleic acids derived therefrom, e.g., pre-mRNA. In some embodiments, the nucleic acid sequence within a Cyclin D2 (CCND2) transcript comprises an exon-intron junction of a Cyclin D2 protein-encoding nucleic acids or naturally occurring variants thereof, and RNA nucleic acids derived therefrom, e.g., pre-mRNA. In some embodiments, for example, when used in research or diagnostics the “nucleic acid sequence within a Cyclin D2 (CCND2) transcript” can be a cDNA or a synthetic oligonucleotide derived from the above DNA or RNA nucleic acid targets. In some embodiments, the human Cyclin D2 protein sequence encoded by the Cyclin D2 pre-mRNA is shown as SEQ ID NO: 1. In some embodiments, the nucleic acid sequence within a Cyclin D2 (CCND2) transcript comprises an untranslated region of a Cyclin D2 protein-encoding nucleic acids or naturally occurring variants thereof, e.g., 5′ UTR, 3′ UTR, or both.

In some embodiments, an oligonucleotide-based therapeutic hybridizes to a region within the introns of a Cyclin D2 transcript, e.g., SEQ ID NO: 1. In certain embodiments, an oligonucleotide-based therapeutic hybridizes to a region within the exons of a Cyclin D2 transcript, e.g., SEQ ID NO: 1. In other embodiments, an oligonucleotide-based therapeutic hybridizes to a region within the exon-intron junction of a Cyclin D2 transcript, e.g., SEQ ID NO: 1.

In some embodiments, the oligonucleotide-based therapeutic comprises a contiguous nucleotide sequence (e.g., 10 to 30 nucleotides in length) that are complementary to a nucleic acid sequence within a Cyclin D2 transcript, e.g., a region corresponding to SEQ ID NO: 1. In some embodiments, the oligonucleotide-based therapeutic comprises a contiguous nucleotide sequence that hybridizes to a nucleic acid sequence, or a region within the sequence, of a Cyclin D2 transcript (“target region”), wherein the nucleic acid sequence corresponds to nucleotides 279 to 1148 of SEQ ID NO: 1.

In some embodiments, the target region corresponds to nucleotides 430 to 447 of SEQ ID NO: 1. In some embodiments, the target region corresponds to nucleotides 430 to 447 of SEQ ID NO: 1±10, ±20, ±30, ±40, ±50, ±60, ±70, ±80, or ±90 nucleotides at the 3′ end and/or the 5′ end.

In some embodiments, the oligonucleotide-based therapeutic of the present disclosure hybridizes to multiple target regions within the Cyclin D2 transcript (e.g., pre-mRNA, SEQ ID NO: 1). In some embodiments, the oligonucleotide-based therapeutic hybridizes to two different target regions within the Cyclin D2 transcript. In some embodiments, the oligonucleotide-based therapeutic hybridizes to three different target regions within the Cyclin D2 transcript. In some embodiments, the oligonucleotide-based therapeutics that hybridizes to multiple regions within the Cyclin D2 transcript (e.g., pre-mRNA, SEQ ID NO: 1) are more potent (e.g., having lower EC50) at reducing Cyclin D2 expression compared to ASOs that hybridizes to a single region within the Cyclin D2 transcript (e.g., pre-mRNA, SEQ ID NO: 1).

In some embodiments, the oligonucleotide-based therapeutic of the disclosure is capable of hybridizing to the target nucleic acid (e.g., Cyclin D2 transcript) under physiological condition, i.e., in vivo condition.

In certain embodiments, the oligonucleotide-based therapeutic disclosed herein is capable of targeting both human and rodent (e.g., mice or rats) Cyclin D2 transcript. Accordingly, in some embodiments, the oligonucleotide-based therapeutic is capable of modulating (e.g., reducing or inhibiting) expression of the Cyclin D2 mRNA or protein both in humans and in rodents (e.g., mice or rats).

In some embodiments, rat Cyclin D2 pre-mRNA can be found at GenBank Accession Number NM_022267.2. In some embodiments, mouse Cyclin D2 pre-mRNA can be found at GenBank Accession Number: NM_009829.3.

### 6.4.2. Antisense Oligonucleotides

In some embodiments, the oligonucleotide-based therapeutics is an antisense oligonucleotide (ASO).

In some embodiments, the ASO mediates reduction or inhibition of Cyclin D2 expression, in part, via nuclease mediated degradation of Cyclin D2. In such cases, the ASO is capable of recruiting a nuclease (e.g., an endonuclease or an endoribonuclease (e.g., RNase H)) to the ASO hybridized to the Cyclin D2 mRNA. Non-limiting examples of ASO designs which operate via nuclease mediated mechanisms are ASOs that typically comprise a region of at least 5 or 6 DNA nucleosides and are flanked on one side or both sides by affinity enhancing nucleosides, for example, gapmers, headmers, and tailmers.

In some embodiments, the RNase H activity of an ASO refers to its ability to recruit RNase H when in a duplex with a complementary RNA molecule (e.g., Cyclin D2 mRNA) and induce degradation of the complementary RNA molecule. In such cases, RNase H activity can be detected using methods available in the art, for example, as described in WO 2001/23613, which is hereby incorporated by reference in its entirety.

In some embodiments, the ASO is capable of reducing Cyclin D2 protein and/or Cyclin D2 transcript expression in the patient or cardiomyocyte by at least about 20% (e.g., by at least about 30%, by least about 40%, by at least about 50%, by at least about 60%, by at least about 70%, by at least about 80%, or by at least 90%) compared to a corresponding patient, cardiomyocyte, or cell not exposed to the ASO.

In some embodiments, the ASO comprises a contiguous polynucleotide sequence of a total of 10, 11, 12, 13, 14, 15, 16, 17, 18, 19, 20, 21, 22, 23, 24, 25, 26, 27, 28, 29, or 30 contiguous nucleotides in length.

In some embodiments, the ASO comprises a polynucleotide sequence having at least about 80%, at least about 85%, at least about 90%, at least about 95%, or about 100% complementarity to the nucleic acid sequence within the Cyclin D2 transcript.

In some embodiments, the ASO comprises the sequence of SEQ ID NO: 5 or SEQ ID NO: 6.

In some embodiments, the ASO is selected from a gapmer, a blockmer, a mixmer, a headmer, a tailmer, a totalmer, a morpholino, a steric blocking ASO, and a CpGs. In some embodiments, the ASO is a gapmer.

In some embodiments, the ASO of the disclosure can comprise a nucleotide sequence which comprises both nucleosides and nucleoside analogs, and can be in the form of a gapmer, a blockmer, a mixmer, a headmer, a tailmer, or a totalmer. Non-limiting examples of configurations of a gapmer, a blockmer, a mixmer, a headmer, a tailmer, or a totalmer that can be used with the ASO of the disclosure are described in U.S. Patent Appl. Publ. No. 2012/0322851, which is herein incorporated by reference in its entirety.

In some embodiments, the term “gapmer” refers to an antisense oligonucleotide (ASO) which comprises a region of RNase H recruiting oligonucleotides (gap) which is flanked 5′ and 3′ by one or more affinity enhancing modified nucleosides (flanks). In some embodiments, the terms “headmers” and “tailmers” are oligonucleotides capable of recruiting RNase H where one of the flanks is missing, i.e., only one of the ends of the oligonucleotide comprises affinity enhancing modified nucleosides. In such cases, headmers, the 3′flank is missing (i.e., the 5′ flank comprises affinity enhancing modified nucleosides). For tailmers, the 5′ flank is missing (i.e., the 3′ flank comprises affinity enhancing modified nucleosides). In some cases, the term “LNA gapmer” is a gapmer oligonucleotide wherein at least one of the affinity enhancing modified nucleosides is an LNA nucleoside. In some cases, the term “mixed wing gapmer” refers to an LNA gapmer wherein the flank regions comprise at least one LNA nucleoside and at least one DNA nucleoside or non-LNA modified nucleoside, such as at least one 2′ substituted modified nucleoside, such as, for example, 2′O-alkyl-RNA, 2′-O-methyl-RNA, 2′-alkoxy-RNA, 2′-O-methoxyethyl-RNA (MOE), 2′-amino-DNA, 2′-Fluoro-RNA, 2′-Fluro-DNA, arabino nucleic acid (ANA), and 2′-Fluoro-ANA nucleoside(s).

In some embodiments, the ASO can be a “chimeric” ASO. In such cases, these ASOs are called “mixmers.” Mixmers include an alternating composition of (i) DNA monomers or nucleoside analog monomers recognizable and cleavable by RNase, and (ii) non-RNase recruiting nucleoside analog monomers. In some embodiments, the ASO can be a “totalmer” ASO. In such cases, a “totalmer” is a single stranded ASO which only comprises non-naturally occurring nucleotides or nucleotide analogs.

In some embodiments, in addition to enhancing affinity of the ASO for the target region, some nucleoside analogs also mediate RNase (e.g., RNaseH) binding and cleavage. In such embodiments, ASOs are designed with a-L-LNA monomers to recruit RNaseH activity. In such cases, gap regions of ASOs containing a-L-LNA monomers consist of fewer monomers recognizable and cleavable by the RNaseH, and more flexibility in the mixmer construction is allowed.

In some embodiments, the ASO of the disclosure is a gapmer and comprises a contiguous stretch of nucleotides (e.g., one or more DNA) which is capable of recruiting an RNase, such as RNaseH (referred to herein in as “region X”), wherein this region is flanked at both 5′ and 3′ by regions of nucleoside analogs, these regions are referred to as 5′ and 3′ flanking regions. In some embodiments, the nucleoside analogs are sugar modified nucleosides (e.g., high affinity sugar modified nucleosides). In some embodiments, the sugar modified nucleosides of the 5′ and 3′ flanking regions enhance the affinity of the ASO for the target nucleic acid (i.e., affinity enhancing 2′ sugar modified nucleosides). In some embodiments, the sugar modified nucleosides are 2′ sugar modified nucleosides, such as high affinity 2′ sugar modifications, such as LNA or 2′-MOE.

In some embodiments, the ASO is a gapmer having the sequence of SEQ ID NO: 5, wherein the gapmer includes flank regions comprising 2′MOE modified nucleobases. In some embodiments, the ASO is a gapmer having the sequence of SEQ ID NO: 6, wherein the gapmer includes flank regions comprising LNA modified nucleobases.

In some embodiments, in a gapmer, the 5′ and 3′ most nucleosides of region X are DNA nucleosides, and are positioned adjacent to nucleoside analogs (e.g., high affinity sugar modified nucleosides) of the 5′ and 3′ flanking regions, respectively. In some embodiments, flanking regions can be further defined by having nucleoside analogs at the end most distant from region X (i.e., at the 5′ end of the 5′ flanking region and at the 3′ end of the 3′ flanking region).

In some embodiments, the ASOs of the present disclosure comprise a nucleotide sequence of formula (5′ to 3′) 5′ flanking region-region X-3′ flanking region, wherein: the 5′ flanking region or a first “wing” sequence comprises at least one nucleoside analog (e.g., 3-5 LNA units); region X comprises at least four consecutive nucleosides (e.g., 4-24 DNA units), which are capable of recruiting RNase (when formed in a duplex with a complementary RNA molecule, such as the pre-mRNA or mRNA target); and the 3′ flanking region or a second “wing” sequence comprises at least one nucleoside analog (e.g., 3-5 LNA units).

In some embodiments, the 5′ flanking region comprises 3-5 nucleotide analogs, such as LNA, region X comprises 6-24 (e.g., 6, 7, 8, 9, 10, 11, 12, 13, or 14) DNA units, and the 3′ flanking region consists of 3 or 4 nucleotide analogs, such as LNA. Such designs include (5′ flanking region—region X-3′ flanking region) 3-14-3, 3-11-3, 3-12-3, 3-13-3, 4-9-4, 4-10-4, 4-11-4, 4-12-4, and 5-10-5. In some embodiments, the ASO has a design of LLLDnLLL, LLLLDnLLLL, or LLLLLDnLLLLL, wherein the L is a nucleoside analog, the D is DNA, and n can be any integer between 4 and 24. In some embodiments, n can be any integer between 6 and 14. In some embodiments, n can be any integer between 8 and 12.

Further gapmer designs are disclosed in WO 2004/046160, WO 2007/146511, and WO 2008/113832, each of which is hereby incorporated by reference in its entirety.

### 6.4.2.1 ASO Modifications

In some embodiments, the ASO comprises at least one non-naturally occurring nucleoside or analogs thereof. “Nucleoside analogs” as used herein are variants of natural nucleosides, such as DNA or RNA nucleosides, by virtue of modifications in the sugar and/or base moieties. In some embodiments, the analogs will have a functional effect on the way in which the ASO works to inhibit expression; for example, by producing increased binding affinity to the target and/or increased resistance to intracellular nucleases and/or increased ease of transport into the cell. In some embodiments, the ASO comprises at least 1, at least 2, at least 3, at least 4, at least 5, at least 6, at least 7, at least 8, at least 9, at least 10, at least 11, at least 12, at least 13, at least 14, at least 15, at least 16, at least 18, at least 19, or at least 20 nucleoside analogs. In some embodiments, the nucleoside analogs in the ASO are the same. In other embodiments, the nucleoside analogs in the ASO are different. In some embodiments, the ASO comprises any of the nucleoside/nucleotide analogs provided herein or any combination thereof.

In some embodiments, the non-naturally occurring nucleoside is a sugar modified nucleoside. In some embodiments, the ASO of the disclosure can comprise one or more nucleosides which have a modified sugar moiety, i.e., a modification of the sugar moiety when compared to the ribose sugar moiety found in DNA and RNA. In some embodiments, ASO comprising a modified sugar moiety are used with the aim of improving certain properties of the ASO, such as affinity and/or nuclease resistance.

In some embodiments, sugar modifications include those where the ribose ring structure is modified, for example, by replacement with a hexose ring (HNA), or a bicyclic ring, which typically have a biradical bridge between the C2′ and C4 carbons on the ribose ring (LNA), or an unlinked ribose ring which typically lacks a bond between the C2′ and C3′ carbons (e.g., UNA). Other sugar modified nucleosides include, for example, bicyclohexose nucleic acids (WO2011/017521) or tricyclic nucleic acids (WO2013/154798), each of which is herein incorporated by reference in its entirety. In some embodiments, modified nucleosides also include nucleosides where the sugar moiety is replaced with a non-sugar moiety, for example in the case of peptide nucleic acids (PNA), or morpholino nucleic acids.

In some embodiments, sugar modifications also include modifications made via altering the substituent groups on the ribose ring to groups other than hydrogen, or the 2′-OH group naturally found in RNA nucleosides. In some embodiments, substituents may, for example be introduced at the 2′, 3′, 4′, or 5′ positions. In some embodiments, nucleosides with modified sugar moieties also include 2′ modified nucleosides, such as 2′ substituted nucleosides. ASOs comprising 2′ substituted nucleosides, have been found to have beneficial properties such as enhanced nucleoside resistance and enhanced affinity.

In some embodiments, the non-naturally occurring nucleoside is a 2′ modified nucleoside. In some embodiments, the non-naturally occurring nucleoside is a 2′ sugar modified nucleoside. In some embodiments, a 2′ sugar modified nucleoside is a nucleoside which has a substituent other than H or —OH at the 2′ position (2′ substituted nucleoside) or comprises a 2′ linked biradical and includes 2′ substituted nucleosides and LNA (2′-4′ biradical bridged) nucleosides. In some embodiments, the 2′ modified sugar may provide enhanced binding affinity (e.g., affinity enhancing 2′ sugar modified nucleoside) and/or increased nuclease resistance to the oligonucleotide.

In some embodiments, the non-naturally occurring nucleoside comprises a 2′-O-alkyl-RNA; 2′-O-methyl RNA (2′-OMe); 2′-alkoxy-RNA; 2′-O-methoxyethyl-RNA (2′-MOE); 2′-amino-DNA; 2′-fluoro-RNA; 2′-fluoro-DNA; arabino nucleic acid (ANA); 2′-fluoro-ANA; or bicyclic nucleoside analog (LNA).

In some embodiments, the sugar modified nucleoside is an affinity enhancing 2′ sugar modified nucleoside. In some embodiments, the affinity enhancing 2′ sugar modified nucleoside is an LNA. In some embodiments, LNA (e.g., a 2′-sugar modified LNA) comprise a linker group (referred to as a biradical or a bridge) between C2′ and C4′ of the ribose sugar ring of a nucleoside (e.g., 2′-4 bridge), which restricts or locks the conformation of the ribose ring. In some cases, a 2′-sugar modified LNA are also termed bridged nucleic acid or bicyclic nucleic acid (BNA). In some embodiments, the locking of the conformation of the ribose is associated with an enhanced affinity of hybridization (duplex stabilization) when the LNA is incorporated into an oligonucleotide for a complementary RNA or DNA molecule. In some embodiments, this can be routinely determined by measuring the melting temperature of the oligonucleotide/complement duplex.

In some embodiments, the LNA is a constrained ethyl nucleoside (cEt), 2′,4′-constrained 21-O-methoxyethyl (cMOE), α-L-LNA, β-D-LNA, 2′-O,4′-C-ethylene-bridged nucleic acids (ENA), amino-LNA, oxy-LNA, thio-LNA, or any combination thereof. In some embodiments, the LNA is β-D-LNA or oxy-LNA.

Non-limiting examples of LNA nucleosides are disclosed in WO 1999/014226, WO 2000/66604, WO 1998/039352, WO 2004/046160, WO 2000/047599, WO 2007/134181, WO 2010/077578, WO 2010/036698. WO 2007/090071, WO 2009/006478, WO 2011/156202, WO 2008/154401, WO 2009/067647, WO 2008/150729, Morita et al, Bioorganic & Med. Chem. Lett. 12, 73-76, Seth et al, J. Org. Chem. 2010, Vol 75(5) pp. 1569-81, and Mitsuoka et al, Nucleic Acids Research 2009, 37(4), 1225-1238, each of which is herein incorporated by reference in its entirety.

In some embodiments, the ASO comprises one or more nucleobases, modified nucleobases, or any combination thereof. The term nucleobase includes the purine (e.g., adenine and guanine) and pyrimidine (e.g., uracil, thymine and cytosine) moiety present in nucleosides and nucleotides which form hydrogen bonds in nucleic acid hybridization. In the context of the present disclosure, the term nucleobase also encompasses modified nucleobases which may differ from naturally occurring nucleobases but are functional during nucleic acid hybridization. In some embodiments, the nucleobase moiety is modified by modifying or replacing the nucleobase. In such cases, “nucleobase” refers to both naturally occurring nucleobases such as adenine, guanine, cytosine, thymidine, uracil, xanthine and hypoxanthine, as well as non-naturally occurring variants. Such variants are for example described in Hirao et al, (2012) Accounts of Chemical Research, Vol. 45, page 2055; and Bergstrom (2009) Current Protocols in Nucleic Acid Chemistry Suppl. 37 1.4.1.

In a some embodiments, the nucleobase moiety is modified by changing the purine or pyrimidine into a modified purine or pyrimidine, such as substituted purine or substituted pyrimidine, such as a nucleobase selected from isocytosine, pseudoisocytosine, 5-methyl-cytosine, 5-thiozolo-cytosine, 5-propynyl-cytosine, 5-propynyl-uracil, 5-bromouracil, 5-thiazolo-uracil, 2-thio-uracil, 2′thio-thymine, inosine, diaminopurine, 6-aminopurine, 2-aminopurine, 2,6-diaminopurine, and 2-chloro-6-aminopurine. In some embodiments, the ASO comprises one or more 5′-methyl-cytosine nucleobases.

The nucleobase moieties may be indicated by the letter code for each corresponding nucleobase, for example, A, T, G, C, or U, wherein each letter may optionally include modified nucleobases of equivalent function. For example, in the exemplified oligonucleotides, the nucleobase moieties are selected from A, T, G, C, and 5-methyl-cytosine. In some cases, for LNA gapmers, 5-methyl-cytosine LNA nucleosides may be used.

In some embodiments, the contiguous nucleotide sequence comprises one or more modified internucleotide or internucleoside linkages.

In some embodiments, the nucleotides (i.e., the monomers) of the ASOs described herein are coupled together via linkage groups. In some embodiments, each monomer is linked to the 3′ adjacent monomer via a linkage group. The person having ordinary skill in the art would understand that, in the context of the present disclosure, the 5′ monomer at the end of an ASO does not comprise a 5′ linkage group, although it may or may not comprise a 5′ terminal group.

In some embodiments, the terms “linkage group” or “internucleoside linkage” are intended to mean a group capable of covalently coupling together two nucleosides. In some embodiments, a linkage group comprises a phosphate group or phosphorothioate group.

In some embodiments, the nucleosides of the ASO or contiguous nucleosides sequence thereof are coupled together via linkage groups. In some embodiments, each nucleoside is linked to the 3′ adjacent nucleoside via a linkage group.

In some embodiments, the internucleoside linkage is modified from a phosphodiester to one that is more resistant to nuclease attack, such as phosphorothioate. In some embodiments, at least 75%, at least 80%, at least 85%, at least 90%, at least 91%, at least 92%, at least 93%, at least 94%, at least 95%, at least 96%, at least 97%, at least 98%, at least 99%, or 100% of internucleoside linkages in an ASO are modified.

Non-limiting examples of ASO design, synthesis, and modifications are as described in: U.S. Patent. Publication No. US 2018/0201936, which is herein incorporated by reference in its entirety.

### 6.4.2.2 Patterns of Modifications

In some embodiments, the Cyclin D2 ASO includes a pattern of one or more modified nucleotides (including modified phosphate linkages). In such cases, the pattern of one or more modified nucleotides is designed to confer stability and/or increased silencing activity. See, e.g., Chernikov et al., Front. Pharmacol., 10:444 (2019), which describes modifications and patterns of modifications that confer stability and/or increase silencing activity.

In some cases, the Cyclin D2 ASOs can be degraded in vivo as a result of its cleavage by endonucleases on pyrimidines and exonucleases from both the 3′ and 5′ ends. Therefore, the ASO can include modifications at cleavage sites to improve dsRNA molecule nuclease resistance to achieve biological activity in vivo. In some embodiments, the introduction of modifications in ASO molecules is determined by the balance between the number of modifications sufficient for ASO molecule to be non-toxic, while retaining interfering activity and nuclease resistance. In some embodiments, introducing a 2′-O-methyl modified nucleotide into the ASO can lead to inhibition of silencing activity if the ASO molecule contains more than two consecutive nucleotides modified to include a 2′-O-methyl modification. In some embodiments, a ASO comprising a 2′-O-methyl modified nucleotide at every second nucleotide does not block silencing activity of the ASO.

In some embodiments, introduction of a 2′-O-methyl modified nucleotide into known cleavages sites preserves the interfering activity of the ASO, increases nuclease resistance, and provides long-term suppression of Cyclin D2. In some embodiments, introduction of a 2′-Fluoro-deoxyadenosine (2′-F) modified nucleotide into known cleavages sites preserves the interfering activity of the ASO, increases nuclease resistance and provides long-term suppression of Cyclin D2. In some embodiments, (2′-F) modified nucleotide are incorporated at terminal nucleotides.

In some embodiments, an ASO comprises alternating 2′O-Me and 2′F modifications. In such cases, these ASO are stable in blood plasma and suppress expression of the target gene.

In some embodiments, introduction of a phosphorothioate (PS) into the ASO preserves the interfering activity of the dsRNA molecule, increases nuclease resistance, and provides long-term suppression of Cyclin D2. In some embodiments, PS are incorporated at terminal nucleotides.

In some embodiments, an ASO comprises one or more 2′O-Me modifications, one or more 2′F modifications, or more or more PS modification, or any combination thereof. In some embodiments, the optimal introduction of 2′O-Me or 2′F modifications for each position in the ASO is determined via in vitro analysis in cardiomycotes (e.g., induced pluripotent stem cell derived cardiomyocytes (iPSC-CM)) or any other suitable system.

In some embodiments, a ASO comprises PS modifications of the 5′ ends in the N-acetylgalactosamine conjugate. In such cases, PS modifications of the 5′ ends in the N-acetylgalactosamine conjugate increase the duration and efficiency of the ASO's inhibitory effect. In some embodiments, conjugates are fully modified at the 2′ positions and stabilized by PS modifications at both the 3′ and 5′ ends of the siRNA.

### 6.4.3. siRNA

In some embodiments, the oligonucleotide-based therapeutics is a double-stranded ribonucleic acid (dsRNA) molecule, for example, a small interfering RNA (siRNA) (also referred to as short interfering RNA), capable of reducing or inhibiting expression of Cyclin D2. In some embodiments, the siRNA is capable of reducing Cyclin D2 protein and/or Cyclin D2 transcript expression in the patient, cardiomyocyte, or cell by at least about 20% (e.g., by at least about 30%, by least about 40%, by at least about 50%, by at least about 60%, by at least about 70%, by at least about 80%, or by at least 90%) compared to a corresponding patient, cardiomyocyte, or cell not exposed to the siRNA.

In some embodiments, the dsRNA comprises a sense strand and an antisense strand. In some embodiments, each strand of the dsRNA includes a sequence between about 10 to about 40 nucleotides (e.g., about 10 to about 35, about 10 to about 30, about 10 to about 20, about 10 to about 15, about 15 to about 40, about 15 to about 35, about 15 to about 30, about 15 to about 25, about 15 to about 20, about 20 to about 40, about 20 to about 35, about 20 to about 30, about 30 to about 25, about 25 to about 40, about 25 to about 35, about 25 to about 30, about 30 to about 40, about 30 to about 35, or about 35 to about 50 nucleotides) in length. In some embodiments, the sense strand and the antisense strand are equal lengths. In some embodiments, the sense strand and the antisense strand are unequal lengths.

In some embodiments, the antisense strand includes a sequence between about 15 to about 40 (or any of the subranges included therein) nucleotides in length.

In some embodiments, the dsRNA molecule (e.g., the siRNA) comprises a sense strand and an antisense strand, each strand having 10 to 40 nucleotides, wherein the antisense strand has sufficient complementarity to the target sequence to mediate RNA interference.

In some embodiments, the siRNA comprises a polynucleotide sequence having at least about 80%, at least about 85%, at least about 90%, at least about 95%, or about 100% complementarity to the nucleic acid sequence within the Cyclin D2 transcript.

In some embodiments, at least 40%, for example at least 45%, at least 50%, at least 55%, at least 60%, at least 65%, at least 70%, at least 75%, or at least 80% of the antisense strand of the dsRNA is present in vivo, for example in a cardiomyocyte, at day 2, 3, 4, 5, 6, 7, 8, 9, 10, 11, 12, 13, 14, 15, 16, 17, 18, 19, 20, 21, 22, 23, 24, or 25 after in vivo administration.

6.4.3.1 siRNA Modifications

In some instances, dsRNA molecules can be degraded in vivo as a result of its cleavage by endonucleases on pyrimidines and exonucleases from both the 3′ and 5′ ends. Therefore, the dsRNA molecule can include modifications including modified nucleotides that increase resistance to endonuclease and exonuclease-mediated degradation.

In some embodiments, the dsRNA molecule (e.g., the siRNA targeting Cyclin D2) comprises at least one (e.g., at least 2, at least 3, at least 4, at least 5, at least 6, at least 7, at least 8, at least 9, or ten or more) modified nucleotides.

In some embodiments, a modified nucleotide includes a modification at the 2′-position, the phosphate linkage, ribose, the nucleobase, or any combination thereof.

In some embodiments, the 2′ position of a nucleotide is modified. In such cases, a nucleotide with a 2′ modification confers improved stability and extends the half-life of the dsRNA molecule. Non-limiting examples of a modified nucleotide include: a 2′-O-methyl modified nucleotide, a 5′-phosphorothioate group modified nucleotide, a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, a non-natural base comprising nucleotide, and a terminal nucleotide linked to a cholesteryl derivative or dodecanoic acid bisdecylamide group. In some embodiments, the modified nucleotide is a 2′-O-methyl modified nucleotide.

In some embodiments, the modified nucleotide comprises a conformationally constrained nucleotide. In some embodiments, the conformationally constrained nucleotide is a Locked Nucleic Acid (LNA). In some embodiments, LNA base(s) improves the stability of a dsRNA molecule and increases the binding affinity to RNA (e.g., the Cyclin D2 transcript).

In some embodiments, an oligonucleotide-based therapeutic includes at least one modification at a phosphate linkage. In some embodiments, an oligonucleotide-based therapeutic includes at least one (e.g., at least 2, at least 3, at least 4, at least 5, at least 6, at least 7, at least 8, at least 9, at least 10, at least 11, at least 12, at least 13. at least 14, at least 15, at least 16, at least 17, at least 18, at least 19, or 20 or more) modified phosphate linkage(s).

Non-limiting examples of modified phosphate linkages include: phosphorothioate linkages, methylphosphonate linkages, ethylphosphonate linkages, boranophosphate linkages, sulfonamide, carbonylamide, phosphorodiamidate, phosphorodiamidate linkages comprising a positively charged side group, phosphorodithioates, aminoethylglycine, phosphotriesters, aminoalkylphosphotriesters; 3′-alkylene phosphonates; 5′-alkylene phosphonates, chiral phosphonates, phosphinates, 3′-amino phosphoramidate, aminoalkylphosphoramidates, thionophosphoramidates; thionoalkyl-phosphonates, thionoalkylphosphotriesters, selenophosphates, 2′-5′ linked boranophosphonate analogs, linkages having inverted polarity, abasic linkages, short chain alkyl linkages, cycloalkyl internucleoside linkages, mixed heteroatom and alkyl or cycloalkyl internucleoside linkages, short chain heteroatomic or heterocyclic internucleoside linkages with siloxane backbones, sulfide, sulfoxide, sulfone, formacetyl linkages, thioformacetyl linkages, methylene formacetyl linkages, thioformacetyl linkages, riboacetyl linkages, alkene linkages, sulfamate backbones, methyleneimino linkages, methylenehydrazino linkages, sulfonate linkages, and amide linkages (as described in WO 2012/145729).

In some embodiments, the phosphate linkage includes a phosphorothioate bond. In such cases, the phosphorothioate (PS) bond substitutes a sulfur atom for a non-bridging oxygen in the phosphate backbone of an oligo. In some embodiments, the phosphorothioate bond modification renders the internucleotide linkage resistant to nuclease degradation. Non-limiting examples of phosphorothioate (PS) bonds are as described in as described in WO 2012/145729, which is herein incorporated by reference in its entirety.

In some embodiments, the phosphate linkage includes a boranophosphate linkage. In such cases, the boranophosphate linkage is more resistant to degradation by endo- and exonucleases relative to a normal phosphodiester linkage.

the sense region, antisense region, or both the sense and antisense strands comprise at least one (e.g., at least two, at least three, or at least four) non-natural phosphodiester linkage. In some embodiments, the sense strand, the antisense strand, or both comprise at least five (e.g., at least six, at least seven, at least eight, or at least nine) non-natural phosphodiester linkages. In some embodiments, the sense strand, the antisense strand, or both comprise at ten (e.g., at least 1, at least 12, at least 13, or at least 14 or more) non-natural phosphodiester linkages.

In some embodiments, a modified nucleotide includes a modification at the ribose ring. Non-limiting examples of modified nucleic acids with modification at the ribose ring include: 6-membered (hexitol (HNA), cyclohexenic (CeNA), and altritol (ANA)); 7-membered rings (oxepanic nucleic acid (ONA)); bicyclic (locked nucleic acids (LNA) or 2′-deoxymethanocarbanucleosides (MCs)); tricyclic (tricyclo-DNA (tc-DNA)), and acyclic (unlocked nucleic acid (UNA)) derivatives). Modified nucleic acids with modifications at the ribose ring can protect siRNAs from the action of nucleases. In some embodiments, a modified nucleic acid with modification at the ribose ring is CeNA. In such cases, CeNA's complementary interaction with RNA stabilizes the duplex, increasing the melting point by 1.5° C. per modified base and increases the oligoribonucleotide resistance to degradation. In some embodiments, a modified nucleic acid with modification at the ribose ring is a bicyclic derivatives (e.g., LNA). In such cases, an LNA increases the melting temperature of siRNA by about 2-8° C. per nucleotide due to the extra cycle between 2′ and 4′ carbon, which fixes the 3′ endo ribose conformation. Additional, ribose ring modifications are as described in Chernikov et al., Front. Pharmacol., 10:444 (2019).

In some embodiments, a modified nucleotide includes a modification at the nucleobase. Non-limiting examples of modified nucleotides having a modification at the nucleobase include: pseudouridine, 2′thiouridine, dihydrouridine, 2,4-difluorobenzene, 4-methylbenzimidazole, hypoxanthine, 7-deazaguanin, N2-alkyl-8-oxoguanine, N2-benzyl-guanine, and 2,6-diaminopurine. In such cases, the modified nucleotides having a modification at the nucleobase are designed to increase the thermal stability of the duplex by increasing the efficiency of the formation of hydrogen bonds with complementary nucleotides on the RNA (e.g., the Cyclin D2 transcript).

In some embodiments, a modified nucleotide includes a modification that is a thermally destabilizing modification. In some embodiments, modifications stabilizing the duplex formed by the 3′ end of the antisense strand and 5′ end of the sense strand and, conversely, modifications destabilizing the duplex formed by the 3′ end of the sense strand and 5′ end of the antisense strand can increase the efficiency of the dsRNA molecule by providing favorable duplex thermal asymmetry.

In some embodiments, the dsRNA molecule includes at least one thermally destabilizing modification of the duplex within the seed region. Non-limiting examples of dsRNA that include at least one thermally destabilizing modification of the duplex within the seed region are as described in WO 2018/098328A1, which is herein incorporated by reference in its entirety.

### 6.4.3.2 Patterns of Modifications

In some embodiments, the dsRNA molecule (e.g., the siRNA targeting Cyclin D2) includes a pattern of one or more modified nucleotides (including modified phosphate linkages). In such cases, the pattern of one or more modified nucleotides is designed to confer stability and/or increased silencing activity. See, e.g., Chernikov et al., Front. Pharmacol., 10:444 (2019), which describes modifications and patterns of modifications that confer stability and/or increase silencing activity. As noted above, in some instances, the dsRNA molecule can be degraded in vivo as a result of its cleavage by endonucleases on pyrimidines and exonucleases from both the 3′ and 5′ ends. Therefore, the dsRNA molecule can include modifications at cleavage sites to improve dsRNA molecule nuclease resistance to achieve biological activity in vivo. However, the introduction of certain modifications in dsRNA molecules are limited by inhibition of its interfering activity and toxicity. In such cases, one parameter affecting RNAi is not only the number of introduced modifications, but also their location in the duplex. In some embodiments, the introduction of modifications in dsRNA molecules is determined by the balance between the number of modifications sufficient for dsRNA molecule to be non-toxic, while retaining interfering activity and nuclease resistance. In some embodiments, introducing a 2′-O-methyl modified nucleotide into the dsRNA can lead to inhibition of RNAi if the dsRNA molecule contains more than two consecutive nucleotides modified to include a 2′-O-methyl modification. In some embodiments, a dsRNA comprising a 2′-O-methyl modified nucleotide at every second nucleotide does not block RNAi.

In some embodiments, introduction of a 2′-O-methyl modified nucleotide into known cleavages sites preserves the interfering activity of the dsRNA molecule, increases nuclease resistance, and provides long-term suppression of Cyclin D2. In some embodiments, known cleavage sites include CA, UA, and UG sites. In such cases, introducing a 2′-O-methyl modified nucleotide to these sites protects the dsRNA molecule from cleavage.

In some embodiments, introduction of a 2′-Fluoro-deoxyadenosine (2′-F) modified nucleotide into known cleavages sites preserves the interfering activity of the dsRNA molecule, increases nuclease resistance, and provides long-term suppression of Cyclin D2. In some embodiments, (2′-F) modified nucleotide are incorporated at terminal nucleotides.

In some embodiments, a dsRNA molecule (e.g., siRNA) comprises alternating 2′O-Me and 2′F modifications. In such cases, these dsRNA are stable in blood plasma and suppress expression of the target gene.

In some embodiments, introduction of a phosphorothioate (PS) into the dsRNA preserves the interfering activity of the dsRNA molecule, increases nuclease resistance, and provides long-term suppression of Cyclin D2. In some embodiments, PS are incorporated at terminal nucleotides.

In some embodiments, a dsRNA molecule (e.g., siRNA) comprises one or more 2′O-Me modifications, one or more 2′F modifications, or more or more PS modification, or any combination thereof. In some embodiments, the optimal introduction of 2′O-Me or 2′F modifications for each position in the siRNA is determined via in vitro analysis in cardiomycotes (e.g., induced pluripotent stem cell derived cardiomyocytes (iPSC-CM)), or any other suitable system.

In some embodiments, a dsRNA molecule (e.g., siRNA) comprises PS modifications of the 5′ ends in the N-acetylgalactosamine conjugate. In such cases, PS modifications of the 5′ ends in the N-acetylgalactosamine conjugate increase the duration and efficiency of the siRNA's inhibitory effect. In some embodiments, conjugates are fully modified at the 2′ positions and stabilized by PS modifications at both the 3′ and 5′ ends of the siRNA.

### 6.5. Conjugation

In some embodiments, the oligonucleotide-based therapeutic is conjugated to a non-nucleotide moiety. In some cases, where an oligonucleotide-based therapeutic is covalently linked to a non-nucleotide moiety the resulting molecule is referred to as a conjugate. In some embodiments, conjugation of an oligonucleotide-based therapeutic of to one or more non-nucleotide moieties may improve the pharmacology of the oligonucleotide-based therapeutic, for example, by affecting the activity, cellular distribution, cellular uptake, or stability of the oligonucleotide-based therapeutic. In some embodiments, the non-nucleotide moieties modify or enhance the pharmacokinetic properties of the oligonucleotide-based therapeutic by improving cellular distribution, bioavailability, metabolism, excretion, permeability, and/or cellular uptake of the oligonucleotide-based therapeutic. In certain embodiments, the non-nucleotide moieties may target the oligonucleotide-based therapeutic to the heart (e.g., a plurality of cells (e.g., cardiomyocytes)). In other embodiments, the non-nucleotide moieties reduce the activity of the oligonucleotide-based therapeutic in non-target cell types, tissues, or organs, e.g., off target activity or activity in non-target cell types, tissues, or organs. Non-limiting examples of non-nucleic acid moieties include: a fatty acid, lipid, peptide, protein, antibody, or nanoparticle.

In some embodiments, oligonucleotide-based therapeutics are conjugated to conjugates where conjugates are as described in WO2013/033230, which is herein incorporated by reference in its entirety.

Conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO 1, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., as described in WO 2014/179620). In some embodiments, the oligonucleotide-based therapeutic is conjugated to a N-acetylgalactosamine (“a N-acetylgalactosamine conjugate”).

In some embodiments, the non-nucleotide moiety (conjugate moiety) is selected from the group consisting of carbohydrates, cell surface receptor ligands, drug substances, hormones, lipophilic substances, polymers, proteins, peptides, toxins (e.g., bacterial toxins), vitamins, viral proteins (e.g., capsids), and combinations thereof.

In some embodiments, an oligonucleotide-based therapeutic is conjugated to at least two (e.g., at least three, at least, at least four, at least five, at least six, at least seven, at least eight, at least nine, or at least ten) non-nucleotide moieties.

In some embodiments, an oligonucleotide-based therapeutic is conjugated to a cardiac targeting peptide. Non-limiting examples of cardiac-targeting peptides are as described in Kim et al. (Mol. Ther. Nucl. Acids, P1024-1032 (2021)), which is herein incorporated by reference in its entirety.

In some embodiments, an oligonucleotide-based therapeutic is activated. In such cases, an activated oligonucleotide-based therapeutic refers to an oligonucleotide-based therapeutic that is covalently linked to a functional moiety that permits covalent linkage of the ASO to one or more conjugated moieties. In some embodiments, a functional moiety will comprise a chemical group that is capable of covalently bonding to the oligonucleotide-based therapeutic via, for example, a 3′-hydroxyl group or the exocyclic NH2 group of the adenine base, a spacer that can be hydrophilic and a terminal group that is capable of binding to a conjugated moiety (e.g., an amino, sulfhydryl or hydroxyl group). In some embodiments, this terminal group is not protected, for example, is an NH2 group. In other embodiments, the terminal group is protected, for example, by any suitable protecting group such as those described in “Protective Groups in Organic Synthesis” by Theodora W. Greene and Peter G. M. Wuts, 3rd edition (John Wiley & Sons, 1999), which is hereby incorporated by reference.

In some embodiments, the oligonucleotide-based therapeutic provided herein are functionalized at the 5′ end in order to allow covalent attachment of the conjugated moiety to the 5′ end of the oligonucleotide-based therapeutic (e.g., on the 5′ end of an ASO or the 5′ end of the antisense strand of a dsRNA molecule). In some embodiments, the oligonucleotide-based therapeutics provided herein can be functionalized at the 3′ end (e.g., on the 3′ end of an ASO or the 3′ end of the antisense strand of a dsRNA molecule). In some embodiments, oligonucleotide-based therapeutics provided herein can be functionalized along the backbone or on the heterocyclic base moiety. In yet other embodiments, the oligonucleotide-based therapeutics provided herein can be functionalized at more than one position independently selected from the 5′ end, the 3′ end, the backbone, and the base.

In some embodiments, activated oligonucleotide-based therapeutics are synthesized by incorporating during the synthesis one or more monomers that is covalently attached to a functional moiety. In some embodiments, activated oligonucleotide-based therapeutics are synthesized with monomers that have not been functionalized, and the oligonucleotide-based therapeutic is functionalized upon completion of synthesis.

In some embodiments, an oligonucleotide-based therapeutic is conjugated to a ligand. In some embodiments, the oligonucleotide-based therapeutic is conjugated to one or more ligands described in WO 2018/044350, which is herein incorporated by reference in its entirety. Methods for linking ligands to the oligonucleotide-based therapeutic are known in the art, for example, as described in WO 2018/044350.

In some embodiments where the oligonucleotide-based therapeutic comprises a dsRNA molecule, the dsRNA molecule can be conjugated to a non-ligand molecule. For example, non-ligand molecules have been conjugated to dsRNAs in order to enhance the activity, cellular distribution or cellular uptake of the dsRNA, and procedures for performing such conjugations are available in the scientific literature. Non-limiting examples on non-ligand moieties are as described in WO 2010/048228, which is herein incorporated by reference in its entirety.

In some embodiments, typical conjugation protocols involve the synthesis of dsRNAs bearing an amino linker at one or more positions of the sequence. In such cases, the amino group is then reacted with the molecule being conjugated using appropriate coupling or activating reagents. In some embodiments, the conjugation reaction may be performed either with the dsRNA molecule still bound to the solid support or following cleavage of the dsRNA molecule in solution phase.

### 6.6. Pharmaceutical Compositions and Dosage

Also provided herein are oligonucleotide-based therapeutics formulated as pharmaceutical compositions. In some embodiments, such compositions comprise a pharmaceutically acceptable diluent, carrier, salt, or adjuvant. In some embodiments, a pharmaceutically acceptable salt comprises a sodium salt, a potassium salt, or an ammonium salt. In some embodiments, any of the oligonucleotide-based therapeutics provided herein can be included in a unit formulation such as in a pharmaceutically acceptable carrier or diluent in an amount sufficient to deliver to a patient a therapeutically effective amount without causing serious side effects in the treated patient. In some cases, in some forms of therapy, serious side effects may be acceptable in terms of ensuring a positive outcome to the therapeutic treatment.

In some embodiments, the formulated pharmaceutical composition (i.e., comprising any of the oligonucleotide-based therapeutics provided herein) may comprise pharmaceutically acceptable binding agents and adjuvants. In some embodiments, capsules, tablets, or pills can contain for example the following compounds: microcrystalline cellulose, gum or gelatin as binders; starch or lactose as excipients; stearates as lubricants; various sweetening or flavoring agents. For capsules, the dosage unit can contain a liquid carrier like fatty oils. Likewise, coatings of sugar or enteric agents can be part of the dosage unit. In some embodiments, the oligonucleotide-based therapeutics formulations can also be emulsions of the active pharmaceutical ingredients and a lipid forming a micellular emulsion.

In some embodiments, the oligonucleotide-based therapeutics may be formulated in a pharmaceutical composition in such a manner to enable co-administration with an anti-cancer therapeutic (e.g., any of the anti-cancer therapeutics described herein).

### 6.6.1. Routes of Administration

Any of the pharmaceutical compositions provided herein or any of the oligonucleotide-based therapeutics provided herein can be administered in a number of ways depending upon whether local or systemic treatment is desired and upon the area to be treated. Non-limiting examples of drug delivery systems for oligonucleotide-based therapeutics include those described in Paunovska et al., Nat. Rev. Gen., 40 (2022). As described in Paunovska, oligonucleotide-based therapeutics can be delivered using synthetic delivery vehicles (e.g., lipids, lipid-based nanoparticles, polymers, and polymer-based nanoparticles). Selection of delivery vehicles depend on factors including, without limitation, therapeutic-related factors (e.g., size, stability, among others), tissue location, duration of desired expression, whether targeting is active or passive or active tissue targeting, among other criteria).

In some embodiments, administration can be (a) oral; (b) pulmonary, e.g., by inhalation or insufflation of powders or aerosols, including by nebulizer; intratracheal, intranasal, (c) topical including epidermal, transdermal, ophthalmic and to mucous membranes including vaginal and rectal delivery; or (d) parenteral including intravenous, intraarterial, subcutaneous, intraperitoneal or intramuscular injection or infusion; or intracranial, e.g., intrathecal, intra-cerebroventricular, or intraventricular, administration. In some embodiments, the oligonucleotide-based therapeutics (or pharmaceutical composition) is administered intravenously, intraperitoneally, orally, topically, or as a bolus injection or administered directly into the target organ. In some embodiments, the oligonucleotide-based therapeutic (or pharmaceutical composition) is administered intracardially or intraventricularly as a bolus injection. In some embodiments, the oligonucleotide-based therapeutic (or pharmaceutical composition) is administered subcutaneously. In some embodiments, the oligonucleotide-based therapeutic (or pharmaceutical composition) is administered orally.

In some embodiments, pharmaceutical compositions and formulations for topical administration can include transdermal patches, ointments, lotions, creams, gels, drops, sprays, suppositories, liquids and powders. Conventional pharmaceutical carriers, aqueous, powder or oily bases, thickeners and the like may be necessary or desirable. Examples of topical formulations include those in which any of the oligonucleotide-based therapeutics provided herein are in admixture with a topical delivery agent such as lipids, liposomes, fatty acids, fatty acid esters, steroids, chelating agents and surfactants. Compositions and formulations for oral administration include but are not limited to powders or granules, microparticulates, nanoparticulates, suspensions or solutions in water or non-aqueous media, capsules, gel capsules, sachets, tablets or minitablets. Compositions and formulations for parenteral, intrathecal, intra-cerebroventricular, or intraventricular administration can include sterile aqueous solutions which can also contain buffers, diluents and other suitable additives such as, but not limited to, penetration enhancers, carrier compounds and other pharmaceutically acceptable carriers or excipients.

In some embodiments, pharmaceutical compositions of the present disclosure include, but are not limited to, solutions, emulsions, and liposome-containing formulations. These compositions may be generated from a variety of components that include, but are not limited to, preformed liquids, self-emulsifying solids and self-emulsifying semisolids. Delivery of oligonucleotide-based therapeutic to the target tissue can be enhanced by carrier-mediated delivery including, but not limited to, cationic liposomes, cyclodextrins, porphyrin derivatives, branched chain dendrimers, polyethylenimine polymers, nanoparticles and microspheres.

The pharmaceutical formulations of the present disclosure, which can conveniently be presented in unit dosage form, can be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

In some embodiments, for parenteral, subcutaneous, intradermal, or topical administration the formulation can include a sterile diluent, buffers, regulators of tonicity and antibacterials. In some embodiments, the oligonucleotide-based therapeutics can be prepared with carriers that protect against degradation or immediate elimination from the body, including implants or microcapsules with controlled release properties. For intravenous administration the carriers can be physiological saline or phosphate buffered saline. International Publication No. WO 2007/031091, further provides suitable pharmaceutically acceptable diluent, carrier and adjuvants—which are hereby incorporated by reference.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Suitable topical formulations include those in which the oligonucleotide-based therapeutics featured in the present disclosure are in admixture with a topical delivery agent such as lipids, liposomes, fatty acids, fatty acid esters, steroids, chelating agents and surfactants.

In some embodiments, the oligonucleotide-based therapeutics are formulated in lipid formulations. Suitable lipids and liposomes include neutral (e.g., dioleoylphosphatidyl DOPE ethanolamine, dimyristoylphosphatidyl choline DMPC, distearoylphosphatidyl choline) negative (e.g., dimyristoylphosphatidyl glycerol DMPG) and cationic (e.g., dioleoyltetramethylaminopropyl DOTAP and dioleoylphosphatidyl ethanolamine DOTMA).

In some embodiments, oligonucleotide-based therapeutics provided herein may be encapsulated within liposomes or may form complexes thereto, in particular to cationic liposomes. Alternatively. oligonucleotide-based therapeutics may be complexed to lipids, in particular to cationic lipids. Suitable fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C1-10 alkyl ester (e.g., isopropylmyristate IPM), monoglyceride, diglyceride or pharmaceutically acceptable salt thereof.

In some embodiments, an oligonucleotide-based therapeutic as provided herein for inhibiting or reducing expression of Cyclin D2 is formulated in a lipid formulation. In some embodiments, a lipid formulation is selected from: a LNP formulation, a LNPO1 formulation, a XTC-SNALP formulation, or a SNALP formulation.

Non-limiting examples of lipid formation are those described in WO 2010/048228A1, which is herein incorporated by reference in its entirety.

### 6.6.2. Dosage and Dosing Schedules

In some embodiments, an oligonucleotide-based therapeutic is administered to a patient at dosage of about 0.01 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.5 mg/kg, about 3.0 mg/kg, about 3.5 mg/kg, about 4.0 mg/kg, about 4.5 mg/kg, about 5.0 mg/kg, about 6.0 mg/kg, about 7.0 mg/kg, about 7.5 mg/kg, about 8.0 mg/kg, about 8.5 mg/kg, about 9.0 mg/kg, about 9.5 mg/kg, or about 10 mg/kg. In some embodiments, a oligonucleotide-based therapeutic is administered to a patient at about 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 44 mg/kg, 45 mg/kg, 46 mg/kg, 47 mg/kg, 48 mg/kg, 49 mg/kg, or 50 mg/kg.

In some embodiments, an oligonucleotide-based therapeutic is administered to a patient at a dosage between 0.01 and 0.2 mg/kg. For example, the dsRNA is administered at a dose of about 0.01 mg/kg, 0.02 mg/kg, 0.3 mg/kg, 0.04 mg/kg, 0.05 mg/kg, 0.06 mg/kg, 0.07 mg/kg 0.08 mg/kg 0.09 mg/kg, 0.10 mg/kg, 0.11 mg/kg, 0.12 mg/kg, 0.13 mg/kg, 0.14 mg/kg, 0.15 mg/kg, 0.16 mg/kg, 0.17 mg/kg, 0.18 mg/kg, 0.19 mg/kg, or 0.20 mg/kg.

In some embodiments, the oligonucleotide-based therapeutic may be administered once daily, or the oligonucleotide-based therapeutic may be administered as two, three, or more sub-doses at appropriate intervals throughout the day or even using continuous infusion or delivery through a controlled release formulation. In that case, the oligonucleotide-based therapeutic contained in each sub-dose must be correspondingly smaller in order to achieve the total daily dosage. In some embodiments, the dosage unit can also be compounded for delivery over several days, e.g., using a conventional sustained release formulation which provides sustained release of the oligonucleotide-based therapeutic over a several day period. Sustained release formulations are well known in the art and are particularly useful for delivery of agents at a particular site, such as could be used with the agents of the present disclosure. In this embodiment, the dosage unit contains a corresponding multiple of the daily dose. Non-limiting examples of dose and dosing schedules for oligonucleotide-based therapeutics are as described in WO 2010/048228, which is herein incorporated by reference in its entirety.

In some embodiments, the effect of a single dose of the oligonucleotide-based therapeutic on Cyclin D2 levels is long lasting, such that subsequent doses are administered at not more than 3, 4, or 5 day intervals, or at not more than 1, 2, 3, or 4 week intervals, or at not more than 5, 6, 7, 8, 9, or 10 week intervals.

The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a oligonucleotide-based therapeutic can include a single treatment or a series of treatments. Estimates of effective dosages and in vivo half-lives for the individual oligonucleotide-based therapeutic provided herein can be made using conventional methodologies or on the basis of in vivo testing using an appropriate animal model, as known in the art.

In some embodiments, the data obtained from cell culture assays and animal studies can be used in formulating a range of dosage for use in humans. The dosage of compositions provided herein lies generally within a range of circulating concentrations that include the ED50 with little or no toxicity. The dosage may vary within this range depending upon the dosage form employed and the route of administration utilized. For any compound used in the methods provided herein, the therapeutically effective dose can be estimated initially from cell culture assays. A dose may be formulated in animal models to achieve a circulating plasma concentration range of the compound or, when appropriate, of the polypeptide product of a target sequence (e.g., achieving a decreased concentration of Cyclin D2 mRNA or protein) that includes the IC50 (i.e., the concentration of the test compound which achieves a half-maximal inhibition of symptoms) as determined in cell culture. In some embodiments, such information can be used to more accurately determine useful doses in humans. In such cases, levels in plasma may be measured, for example, by high performance liquid chromatography.

In some embodiments, the oligonucleotide-based therapeutics provided herein can be administered in combination with other known agents effective in treatment of pathological processes mediated by target gene expression. In such cases, the administering physician can adjust the amount and timing of the oligonucleotide-based therapeutic's administration on the basis of results observed using standard measures of efficacy known in the art or described herein.

### 6.6.3. Additional Methods for Delivering Oligonucleotide-Based Therapeutics

In some embodiments, oligonucleotide-based therapeutics are administered to a patient or contacted with a cell (e.g., a cardiomyocyte) using a vector. In some embodiments, the vector is an expression vector (e.g., a DNA plasmid vector) or a viral vectors. In some embodiments, the vector is a viral vector. In cases where the oligonucleotide-based therapeutics is delivered using a vector, appropriate regulatory elements (e.g., promoters, enhancers. polyA signals, or any combination thereof) are used to drive expression of the oligonucleotide-based therapeutics.

Any viral vector capable of accepting the coding sequences for the oligonucleotide-based therapeutics to be expressed can be used. In some embodiments, the tropism of viral vectors can be modified by pseudotyping the vectors with envelope proteins or other surface antigens from other viruses, or by substituting different viral capsid proteins, as appropriate. Non-limiting examples of viral vectors include: lentivirus, retrovirus, gammaretroviruses, adeno-associated virus, adenovirus, helper-dependent adenovirus, sendai virus, or a baculovirus.

In some embodiments, lentiviral vectors are used to deliver the oligonucleotide-based therapeutics. In such cases, the lentiviral vector can be pseudotyped with surface proteins from vesicular stomatitis virus (VSV), rabies, Ebola, Mokola, and the like.

In some embodiments, AAV vectors are used to deliver the oligonucleotide-based therapeutics. In such cases, a particular AAV capsid protein serotype can be selected based on its ability to infect the heart (e.g., cardiomyocytes within the heart). In some embodiments, AAV serotype can be selected based, in part, on the AAV expression as performed in Zincarelli et al., Mol. Ther. 16(6): 1073-1080 (2008), which is herein incorporated by reference in its entirety. In some embodiments, an AAV9 capsid is used to target the oligonucleotide-based therapeutics to the heart. In some embodiments, an AA1, AAV4, AAV6, or AAV8 are used to target the oligonucleotide-based therapeutics to the heart. Techniques for constructing AAV vectors which express different capsid protein serotypes are within the skill in the art; see, e.g., Rabinowitz et al., J. Virol. 76:791-801 (2002), which is herein incorporated by reference in its entirety.

Selection of recombinant viral vectors suitable for use in the present disclosure, methods for inserting nucleic acid sequences for expressing the dsRNA into the vector, and methods of delivering the viral vector to the cells of interest are within the skill in the art. See, for example, Dornburg R (1995), Gene Therap. 2: 301-310; Eglitis M A (1988), Biotechniques 6: 608-614; Miller A D (1990), Hum Gene Therap. 1: 5-14; Anderson W F (1998), Nature 392: 25-30; and Rubinson D A et al., Nat. Genet. 33: 401-406, the entire disclosures of which are herein incorporated by reference. Viral vectors can be derived from AV and AAV. In one embodiment, the dsRNA featured in the present disclosure is expressed as two separate. complementary single-stranded RNA molecules from a recombinant AAV vector having, for example, either the U6 or H1 RNA promoters, or the cytomegalovirus (CMV) promoter. A suitable AV vector for expressing the dsRNA featured in the invention, a method for constructing the recombinant AV vector, and a method for delivering the vector into target cells, are described in Xia H et al. (2002), Nat. Biotech. 20: 1006-1010.

Suitable AAV vectors for expressing the oligonucleotide-based therapeutic featured in the present disclosure, methods for constructing the recombinant AV (or AAV) vector, and methods for delivering the vectors into target cells are described in Samulski R et al. (1987), J. Virol. 61: 3096-3101; Fisher K J et al. (1996), J. Virol, 70: 520-532; Samulski R et al. (1989), J. Virol. 63: 3822-3826; U.S. Pat. Nos. 5,252,479; 5,139,941; International Patent Application No. WO 94/13788; and International Patent Application No. WO 93/24641, the entire disclosures of which are herein incorporated by reference. The promoter driving expression of an oligonucleotide-based therapeutic in either a DNA plasmid or viral vector featured in the present disclosure may be a eukaryotic RNA polymerase I {e.g., ribosomal RNA promoter), RNA polymerase II {e.g., CMV early promoter or actin promoter or U1 snRNA promoter) or generally RNA polymerase III promoter {e.g., U6 snRNA or 7SK RNA promoter) or a prokaryotic promoter, for example the T7 promoter, provided the expression plasmid also encodes T7 RNA polymerase required for transcription from a T7 promoter. The promoter can also direct transgene expression to the pancreas (see, e.g., the insulin regulatory sequence for pancreas (Bucchini et al., 1986, Proc. Natl. Acad. Sci. USA 83:2511-2515)).

In addition, expression of the transgene can be precisely regulated, for example, by using an inducible regulatory sequence and expression systems such as a regulatory sequence that is sensitive to certain physiological regulators, e.g., circulating glucose levels, or hormones (Docherty et al., 1994, FASEB J. 8:20-24). Such inducible expression systems. suitable for the control of transgene expression in cells or in mammals include regulation by ecdysone, by estrogen, progesterone, tetracycline, chemical inducers of dimerization, and isopropyl-beta-D1-thiogalactopyranoside (EPTG). A person skilled in the art would be able to choose the appropriate regulatory/promoter sequence based on the intended use of the dsRNA transgene.

In some embodiments, recombinant vectors capable of expressing oligonucleotide-based therapeutics molecules are delivered as provided herein and persist in target cells. In other embodiments, viral vectors can be used that provide for transient expression of oligonucleotide-based therapeutics. In such cases, vectors can be repeatedly administered as necessary. Once expressed, the oligonucleotide-based therapeutics bind to target RNA and modulate its function or expression.

In some embodiments, delivery of oligonucleotide-based therapeutics expressing vectors can be systemic, such as by intravenous or intramuscular administration, by administration to target cells ex-planted from the patient followed by reintroduction into the patient, or by any other means that allows for introduction into a desired target cell. Successful introduction of vectors into host cells can be monitored using various known methods. For example, introduction can be signaled with a reporter, such as a fluorescent marker, such as Green Fluorescent Protein (GFP).

In some embodiments, the oligonucleotide-based therapeutics are administered to patient or a contacted with a cardiomyocyte using exosome that include the oligonucleotide-based therapeutic, for example, as described in U.S. Patent Publication No. US 2020/0308587, which is herein incorporated by reference in its entirety.

### 6.7. Kits

This disclosure also features kits comprising an oligonucleotide-based therapeutic (e.g., any oligonucleotide-based therapeutic provided herein) and that can be used to perform any of the methods described herein.

In certain embodiments, a kit comprises at least one oligonucleotide-based therapeutic in one or more containers. In some embodiments, the kits contain all of the components necessary and/or sufficient to perform a detection assay, including all controls, directions for performing assays, and any necessary software for analysis and presentation of results. One skilled in the art will readily recognize that the disclosed oligonucleotide-based therapeutic can be readily incorporated into one of the established kit formats which are well known in the art.

### 7. EXAMPLES

### 7.1. Methods

### Cells for In Vitro Studies

Neonatal rat ventricular cardiomyocytes (NRVMs) isolated from 7-day old Sprague Dawley Rats (P7-NRVMs) were used to investigate hypertrophy, metabolism, and metabolic gene expression in vitro.

Human induced Pluripotent Stem Cell derived cardiomyocytes (hiPSC-CMs) acquired from Cellular Dynamics International (CDI) or cardiomyocytes derived from hiPSC cell line AICS-0060-027, were used to investigate hypertrophy, metabolic gene expression. lipid accumulation, and lipotoxicity in vitro.

### NRVM Isolation and Seeding

NRVMs from 7-day old Sprague Dawley rats were isolated as described previously (Zebrowski et al., 2015) and seeded 2 days prior to experimentation.

### Media Formulation for Seeding NRVMs:

DMEM/F12+Glutamax (Thermofisher), 5% Horse Serum (Thermofisher), 10 micromolar AraC (Sigma). 1% PenStrep (Thermofisher).

NRVMs were plated in 24-well plates on glass coverslips (Thermo Scientific, Menzel-Glaser. 13 mm #0), or 96-well flat bottom plates, coated with Fibronectin (Sigma, F1141).

hiPSC-CM (CDI) Culturing

Human induced-Pluripotent Stem Cell-derived cardiomyocytes (hiPSC-CMs) were acquired from Cellular Dynamics International, Inc. (CDI) (iCell Cardiomyocytes). hiPSC-CMs were seeded and cultured according to manufacturer's directions (see cellulardynamics.com/assets/CDI_iCellCardiomyocytes_UG.pdf). hiPSC-CMs were plated in 24-well plates on glass coverslips (Thermo Scientific, Menzel-Glaser. 13 mm #0) coated with Geltrex (Thermofisher) for at least 2 hours at room temperature. hiPSC-CMs were seeded with M1001 iCell Cardiomyocytes Plating Medium. After 2 days, media was changed to M1003 iCell Cardiomyocytes Maintenance Medium.

hiPSC-CM (AICS-0060-027 Derived) Culturing

Human induced-Pluripontent Stem Cell-derived cardiomyocytes (hiPSC-CMs) were derived from hiPSC line AICS-0060-027 were used for the lipotoxicity assay as measured by % 7-ADD. AICS-0060-027-derived cardiomyocytes were derived and cultured as described previously, with the exception of being differentiated for 30 days (protocol can be found at doi.org/10.1093/cvr/cvab311).

### Induction of Pathological Stress in Cardiomyocytes In Vitro

For induction of pathological stress in P7-NRVMs and hiPSC-CMs: after seeding (i.e. 2-4 days in Seeding Media), media was switched to Starvation Media.

### Formulation for NRVM Starvation Media:

DMEM+GlutaMax (Thermofisher), 10 micromolar AraC (Sigma), and 1% PenStrep (Thermofisher).

Formulation for hiPSC-CM Starvation Media:

DMEM+GlutaMax (Thermofisher) and 10 micromolar AraC (Sigma).

One day after starvation media, cardiomyocytes were treated with either Isoproterenol (ISO) at 10 micromolar (μM) or Angiotensin II (ANGII) at 1 micromolar (μM) (added to Starvation Media) to mimic pathological cardiac stress in vitro. Unless otherwise stated, 30 minutes prior to stimulation with either ISO or ANGII, cardiomyocytes were transfected with oligonucleotides (e.g., Cyclin D2 ASO, scramble ASO, Cyclin D2 siRNA, or scramble siRNA) at a final concentration of 40 nM using RNAi MAX following manufacture's transfection protocols. Unless otherwise stated, cardiomyocytes were analyzed 2 days after stimulation with either ISO or ANGII.

### RNA Isolation and RT-qPCR for Gene Expression Measurements

Total RNA was isolated with the TRIzol® Reagent (ThermoFisher Technologies) according to the manufacturer's instructions. The quantity and purity of RNA were measured using the Synergy HTX Multi-Mode Reader (BioTek). mRNA was reverse transcribed to cDNA by using Maxima™ H Minus cDNA Synthesis Master Mix Kit, with dsDNase (ThermoFisher Technologies) to remove potential DNA contamination. Primers were purchased from Thermo Fisher Scientific. qPCR was performed using SYBR green chemistry, GoTaq® qPCR and RT-qPCR Systems (Promega) and a Quant Studio 12K Flex instrument (ThermoFisher Technologies) by following the manufacturer's protocol. Triplicate PCR reactions were performed. Comparative ΔCt method was used by collecting the mean cycle threshold (Ct) values of triplicate wells for each sample and the expression value was normalized to the Ct values for endogenous control glyceraldehyde 3-phosphate dehydrogenase (GAPDH). Relative gene expression analysis was determined according to static.thermoscientific.com/images/D21497˜.pdf.

### Generation of Oligonucleotide-Based Therapeutics for Modulating Cyclin D2 Expression

Antisense oligonucleotides for modulating Cyclin D2 expression were adapted from U.S. Pat. No. 6,492,173, which is herein incorporated by reference in its entirety.

siRNA oligonucleotide for modulating rat Cyclin D2 expression was purchased from Qiagen (catalogue number, SI01496243).

Transfection of siRNAs and ASOs

All siRNAs and ASOs were transfected into cardiomyocytes at a final concentration of 40 nM using Lipofectamine RNAi MAX transfection reagent (ThermoFisher Scientific) according to manufacturer's protocols.

### Hypertrophy Assay

Analysis was conducted via immunohistochemistry to identify cardiomyocytes and ImageJ software to measure cell area. Antibodies used for immunohistochemical designation of cardiomyocytes for analysis include: anti-sarcomeric alpha Actinin antibody (mouse, 1:500, Abeam, EA-53); Donkey anti-Mouse IgG (H+L) Cross-absorbed secondary antibody, and Alexa Flour 594, 17 (1:500, ThermoFisher Scientific, A-21203). DAPI was used as a counter stain. Images for analysis were captured on Leica DM6B-02 microscope with DFC9000GT camera. Only mononucleated cardiomyocytes were scored. Cell area was quantitively determined using NIH Image J.

### Lipotoxicity Assay

hiPSC-CMs (30 day old, derived from AICS-0060-027) seeded on glass-bottom plate were starved with DMEM (low glucose, GlutaMax, Thermofisher) for 24 hrs followed by adding 1 μM Ang II and transfection of 40 nM SCRAMBLE or Cyclin D2 ASO (D2ASO-2) by Lipofectamine RNAiMax reagent (Thermofisher). 24 hrs after transfection of ASOs, BSA or Palmitate (PA)-BSA (Agilent Seahorse) was added to a final PA concentration of 0 or 800 μM and incubated for 24 hrs. Media was removed and cells were stained with 1 g/ml 7-aminoactinomycin D (7-AAD, Thermofisher) and 2 μg/ml Hoechst33342 (Thermofisher) for 5 mins followed by washing with DPBS twice. Whole plate was imaged with Leica THUNDER imager and %7-AAD positive cell was analysed by FIJI.

### Fatty Acid Accumulation Assay

hiPSC-CMs (iCell Cardiomyocytes from Cellular Dynamics Inc (CDI)) were seeded on glass-bottom plate were starved with DMEM (low glucose, GlutaMax, Thermofisher) for 24 hrs followed by adding 1 μM ANG II and transfection of 40 nM SCRAMBLE ASO or Cyclin D2 ASO(1) (SEQ ID No. 5) or Cyclin D2 ASO(2) (SEQ ID No. 7) by Lipofectamine RNAiMax reagent (Thermofisher). BODIPY-C16 was added to the cells at either the same time when ANGII was added or 24 hours later. 36 hours after ANGII was added, cells were fixed and analyzed. Antibodies used for immunohistochemical designation of cardiomyocytes for analysis include: anti-sarcomeric alpha Actinin antibody (mouse, 1:500, Abeam, EA-53); Donkey anti-Mouse IgG (H+L) Cross-absorbed secondary antibody, and Alexa Flour 594, 17 (1:500, ThermoFisher Scientific, A-21203). DAPI was used as a counter stain. Images for analysis were captured on Leica DM6B-02 microscope with DFC9000GT camera.

The formulation of using BODIPY C16 in culture medium is as described in the table below:

| | Stock | 100x | Working |
|---|---|---|---|
| | Concentration | mixture | Concentration |
| | milliM | microM | microM |
| BODIPY-C16 | 10 | 3000 | 30 |
| BSA | 3 | 1500 | 15 |
| L-carnitine | 2000 | 200000 | 2000 |

Mixture is added to cell culture medium directly as a supplement.

### Seahorse Assay for Measurements of Fatty Acid Oxidation in NRVMs

Measurement of cellular Oxygen Consumption and determination of Maximal Respiration and Spare Respiratory Capacity (SRC) was done using a Seahorse bioscience XF96 analyzer in 96 well plates and a XF Palmitate Oxidation Stress Test (Agilent) following established protocols developed by Agilent and others (star-protocols.cell.com/protocols/1653; Angelini et al, STAR Protocols, May 20, 2022). Cardiomyocytes were subjected to pathological stress, as described in methods herein, prior to the Seahorse assay.

### Lipotoxicity Assay

hiPSC-CM seeded on glass-bottom plate was starved with DMEM (low glucose, GlutaMax, Thermofisher) for 24 hrs followed by adding 1 μM Ang II and transfection of 40 nM SCRAMBLE or Cyclin D2 ASO by Lipofectamine RNAiMax reagent (Thermofisher). 24 hrs after transfection of ASOs, BSA or Palmitate (PA)-BSA (Agilent Seahorse) was added to a final PA concentration of 0-800 μM and incubated for 24 hrs. Media was removed and cells were stained with 1 g/ml 7-aminoactinomycin D (7-AAD, Thermofisher) and 2 g/ml Hoechst33342 (Thermofisher) for 5 mins followed by washing with DPBS twice. Whole plate was imaged with Leica THUNDER imager and %7-AAD positive cell was analysed by FIJI.

Primer sequences: Table discloses SEQ ID NOS 8-15, respectively, in order of appearance.

### PRIMERS for RT-PCR

| GENE | SPECIES | DIRCTION | SEQUENCE |
|---|---|---|---|
| MCAD | RAT | FORWARD | GTC GCC CCA GAC TAC GAT AA |
| MCAD | RAT | REVERSE | GCC AAG ACC ACC ACA ACT CT |
| MCPT1 | RAT | FORWARD | AAG AAC ACG AGC CAA CAA GC |
| MCPT1 | RAT | REVERSE | TAC CAT ACC CAG TGC CAT CA |
| Cyclin D2 | RAT | FORWARD | CAGAAGGACATCCAGCCGTAC |
| Cyclin D2 | RAT | REVERSE | TCGGGACTCCAGCCAAGAA |
| Human mCPT-1 | HUMAN | FORWARD | CTTTGACCGACACCTGTTTGC |
| Human mCPT-1 | HUMAN | REVERSE | TGGTGGACAGGATGTTGTGGTT |

### 7.2. Example 1. siRNA-Mediated Inhibition of Cyclin D2 Suppressed Hypertrophy in NRVMs

A siRNA targeting Cyclin D2 was used to assess whether reducing Cyclin D2 expression levels suppresses cardiomyocyte hypertrophy in response to pathologic stimuli Isoproterenol (ISO) and Angiotensin II (ANGII). In particular, cardiomyocytes (Neonatal rat ventricular cardiomyocytes (NRVMs)) were subjected to pathological stress, transfected with a Cyclin D2 siRNA or a Scramble (i.e. control) siRNA and cardiomyocyte hypertrophy was measured by determining the cell area of individual cardiomyocytes.

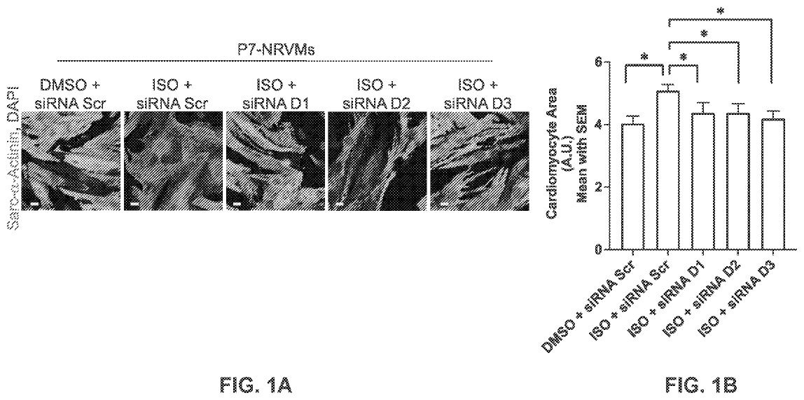

FIGS. 1A-1B show statistically significant differences in NRVM area between NRVMs treated with ISO and transfected with Scramble siRNA and NRVMs treated with ISO and transfected with Cyclin D2 siRNA. NRVMs treated with ISO and transfected with Cyclin D2 siRNA (i.e., siRNA D1, siRNA D2, or siRNA D3) had similar mean cardiomyocyte area as the cardiomyocytes transfected with Scramble siRNA only. In summary, FIGS. 1A-1B show that under conditions of pathologic stress (i.e. ISO), treating with a cyclin D2 oligonucleotide inhibitor (i.e., siRNA D1, siRNA D2, or siRNA D3) inhibits NRVM hypertrophic growth.

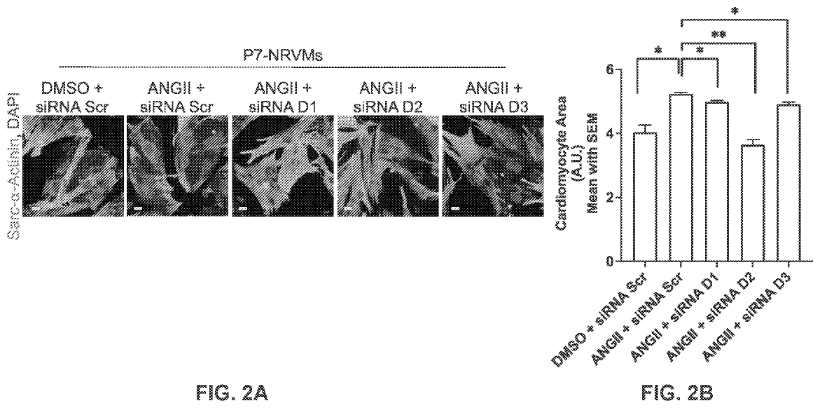

FIGS. 2A-2B show statistically significant differences in NRVM area between NRVMs treated with ANGII and transfected with Scramble siRNA and NRVMs treated with ANGII and transfected with Cyclin D2 siRNA. NRVMs treated with ANGII and transfected with Cyclin D2 siRNA (i.e., siRNA D1, siRNA D2, or siRNA D3) had similar mean NRVM area as the cardiomyocytes transfected with Scramble siRNA only. Taken together, FIG. 2 shows that under conditions of pathologic stress (i.e. ANGII), Cyclin D2 oligonucleotide inhibitor (i.e. siRNA D1, siRNA D2, or siRNA D3) inhibits NRVM hypertrophic growth.

Overall, these results show that inhibiting or reducing Cyclin D2 expression using an oligonucleotide (e.g., an siRNA that targets Cyclin D2) can prevent and/or inhibit both ISO and ANGII induced cardiomyocyte hypertrophy.

### 7.3. Example 2. ASO-Mediated Inhibition of Cyclin D2 Suppressed Hypertrophy in NRVMs

In this example, an antisense oligonucleotide (ASO) LNA-modified gapmer having the sequence of SEQ ID NO: 6 was used in a method for reducing or inhibiting Cyclin D2 expression.

For these experiments, an ASO designed to inhibit or reduce Cyclin D2 expression was used to assess whether reducing Cyclin D2 expression levels suppresses cardiomyocyte hypertrophy in response to pathologic stimuli in the form of Isoproterenol (ISO) or Angiotensin II (ANGII). In particular, cardiomyocytes (Neonatal rat ventricular cardiomyocytes (NRVMs)) were subjected to pathological stress (e.g. ISO or ANGII) and transfected with a Cyclin D2 ASO and cardiomyocyte hypertrophy was measured by determining the cell area of individual cardiomyocytes.

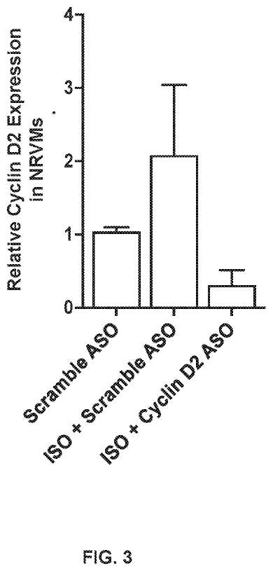

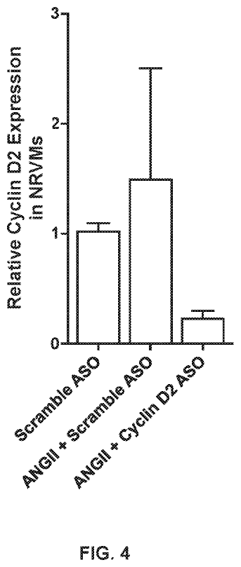

The results in FIG. 3 and FIG. 4 show that the Cyclin D2 ASO is capable of suppressing Cyclin D2 expression. FIG. 3 shows that when cells are under conditions of pathologic stress (i.e., ISO-induced pathological stress), treating the cells with a Cyclin D2 oligonucleotide inhibitor (i.e., ASO having a sequence of SEQ ID NO: 6) inhibited Cyclin D2 expression in NRVMs. FIG. 4 shows that when cells are under conditions of pathologic stress (i.e., ANGII-induced pathological stress), treating the cells with a Cyclin D2 oligonucleotide inhibitor (i.e. ASO, SEQ ID NO: 6) inhibited Cyclin D2 expression in NRVMs. FIGS. 3-4 show that when cells are under conditions of pathologic stress (i.e., ISO (FIG. 3) or ANGII (FIG. 4)), treating the cells with a Cyclin D2 oligonucleotide inhibitor (i.e., ASO having the sequence of SEQ ID NO: 6) inhibited Cyclin D2 expression.

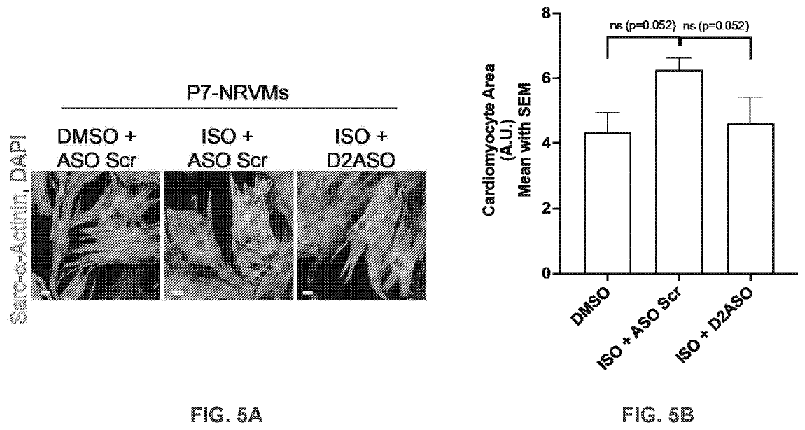

FIGS. 5A-B show the effect of an LNA gapmer antisense oligonucleotide (ASO) targeting Cyclin D2 in neonatal rat ventricular cardiomyocytes (isolated from 7-day old pups) (“P7-NVRMs”). FIG. 5A shows immunofluorescence images of P7-NVRMS stained with an anti-Sarcomeric Alpha Actinin and DAPI. FIG. 5B show statistically significant differences in NRVM area between NRVMs treated with ISO and transfected with Scramble ASO and NRVMs treated with ISO and transfected with Cyclin D2 ASO. NRVMs treated with ISO and transfected with Cyclin D2 siRNA had similar mean cardiomyocyte area as the cardiomyocytes transfected with Scramble ASO only. Overall. FIGS. 5A-5B shows that when cells are under conditions of pathologic stress (i.e., ISO-induced pathological stress), treating the cells with a Cyclin D2 oligonucleotide inhibitor (i.e., ASO having the sequence of SEQ ID NO: 6) inhibited NRVM hypertrophic growth.

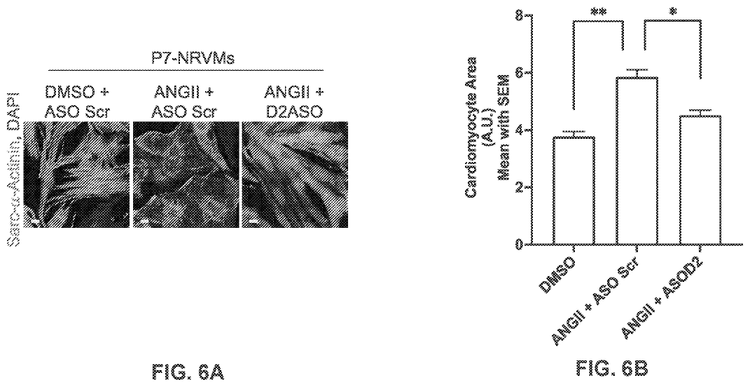

FIGS. 6A-B show the effect of an LNA gapmer antisense oligonucleotide (ASO) targeting Cyclin D2 in neonatal rat ventricular cardiomyocytes (isolated from 7-day old pups) (“P7-NVRMs”). FIG. 6A shows immunofluorescence images of P7-NVRMS stained with an anti-Sarcomeric Alpha Actinin and DAPI. FIG. 6B shows statistically significant differences in NRVM area between NRVMs treated with ANGII and transfected with Scramble ASO and NRVMs treated with ANGII and transfected with Cyclin D2 ASO. NRVMs treated with ANGII and transfected with Cyclin D2 ASO had similar mean NRVM area as the cardiomyocytes transfected with Scramble ASO only. Overall, FIGS. 6A-6B show that when cells are under conditions of pathologic stress (e.g., ANGII-induced pathological stress), treating the cells with a Cyclin D2 oligonucleotide inhibitor (e.g., ASO having the sequence of SEQ ID NO: 6) inhibited NRVM hypertrophic growth.

In summary, these results show that inhibiting or reducing Cyclin D2 expression using an oligonucleotide (e.g., an ASO (e.g., SEQ ID NO: 6) that targets Cyclin D2 can prevent and/or inhibit both ISO and ANGII induced cardiomyocyte hypertrophy.

### 7.4. Example 3. ASO-Mediated Inhibition of Cyclin D2 Suppressed Hypertrophy in hiPSC-CMs

In this example, an antisense oligonucleotide comprising the sequence of SEQ ID NO: 5 was used in a method for reducing or inhibiting Cyclin D2 expression.

For these experiments, an ASO designed to inhibit or reduce Cyclin D2 expression was used to assess whether reducing Cyclin D2 expression levels suppresses cardiomyocyte hypertrophy in response to pathologic stimuli Isoproterenol (ISO). In particular, hiPSC-CMs (procured from CDI) were subjected to pathological stress (ISO) and transfected with a Cyclin D2 ASO and cardiomyocyte hypertrophy was measured by determining the cell area of individual cardiomyocytes.

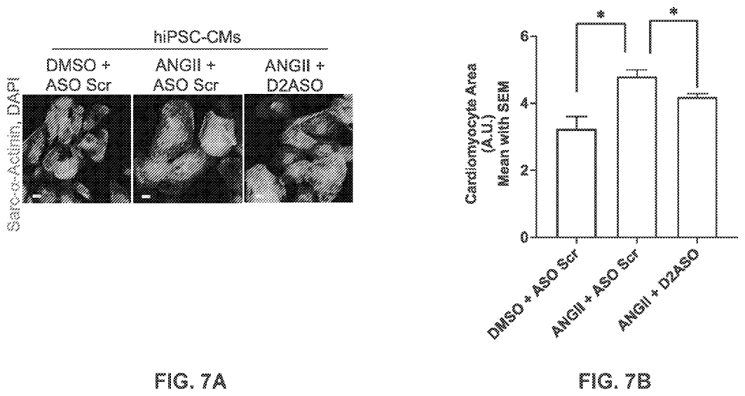

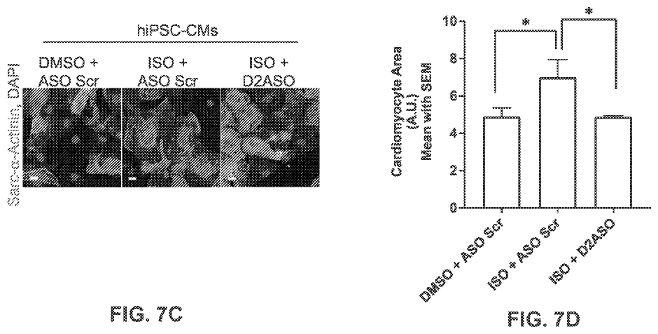

FIGS. 7A-7B show the effect of an LNA gapmer antisense oligonucleotide (ASO) targeting Cyclin D2 in human induced-pluripotent stem cell derived cardiomyocytes (hiPSC-CMs). FIG. 7A shows immunofluorescence images stained with an anti-Sarcomeric Alpha Actinin and DAPI. FIG. 7B shows statistically significant differences in hiPSC-CM area between hiPSC-CMs treated with ANGII and transfected with Scramble ASO and hiPSC-CMs treated with ISO and transfected with Cyclin D2 ASO. hiPSC-CMs treated with ANGII and transfected with Cyclin D2 ASO had similar mean cardiomyocyte area as the hiPSC-CMs transfected with Scramble ASO only. Overall, FIGS. 7A-7B shows that when cells are under conditions of pathologic stress (e.g., ANGII-induced pathological stress), treating the cells with a Cyclin D2 oligonucleotide inhibitor (e.g., an ASO having the sequence of SEQ ID NO: 5) inhibited NRVM hypertrophic growth.

FIGS. 7C-7D show the effect of an LNA gapmer antisense oligonucleotide (ASO) targeting Cyclin D2 in human induced-pluripotent stem cell derived cardiomyocytes (hiPSC-CMs). FIG. 7C shows immunofluorescence images stained with an anti-Sarcomeric Alpha Actinin and DAPI. FIG. 7D shows statistically significant differences in hiPSC-CM area between hiPSC-CMs treated with ISO and transfected with Scramble ASO and hiPSC-CMs treated with ISO and transfected with Cyclin D2 ASO. hiPSC-CMs treated with ISO and transfected with Cyclin D2 ASO had similar mean cardiomyocyte area as the hiPSC-CMs transfected with Scramble ASO only. Overall, FIGS. 7C-7D shows that when cells are under conditions of pathologic stress (e.g., ISO-induced pathological stress), treating the cells with a Cyclin D2 oligonucleotide inhibitor (e.g., an ASO having the sequence of SEQ ID NO: 5) inhibited NRVM hypertrophic growth.

In summary, these results show that inhibiting or reducing Cyclin D2 expression using an oligonucleotide (e.g., an ASO that target Cyclin D2) can prevent/inhibit ISO induced cardiomyocyte hypertrophy in hiPSC-CMs.

### 7.5. Example 4. Cardiomyocytes Treated with Cyclin D2 ASO Preserve Fatty Acid Oxidation Following Pathologic Stimuli (ISO and ANGII)

This experiment was designed to test whether knockdown of Cyclin D2 induced gene expression of key genes required for FAO in cardiomyocytes following exposure to pathological stimuli. In the adult 95% of ATP generated in the heart is due to either glucose, or fatty acid, oxidation (40-70% of which is FAO) with only 5% of ATP generated by Glycolysis. In response to pathological stimuli-induced heart failure in postnatal cardiomyocytes, the cells revert to a fetal metabolic profile whereby the primary source of energy production is Glycolysis not FAO.

For these experiments, NRVMs and hiPSC-CMs were subjected to pathological stress with either ISO or ANGII, transfected with either Scramble (i.e. Control) or Cyclin D2 oligonucleotides (e.g., siRNA targeting Cyclin D2 or ASO targeting Cyclin D2), and then assessed for MCPT1 and MCAD mRNA expression using RT-PCR. MCPT1 and MCAD are genes required for cardiomyocytes to conduct FAO. As such, these genes serves as markers for a cell's potential of, or capability to utilize, FAO.

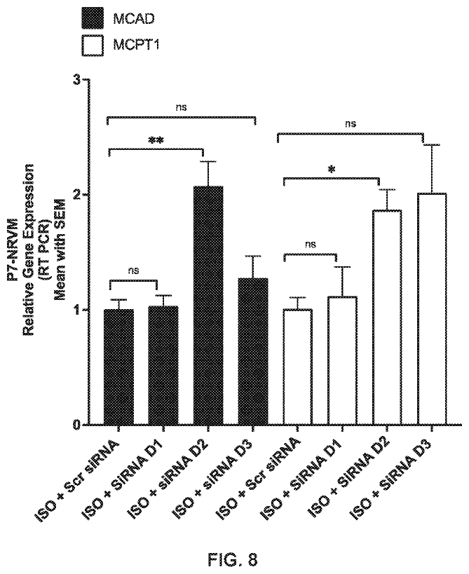

The results of the MCPT1 and MCAD mRNA expression measurements are provided in FIGS. 8-12. For each condition, results from three biological replicates were provided. Statistically significant differences were determined by student's t-test comparing two conditions with P-values indicated with asterisks, * <0.05, and ns=not significant.

FIG. 8 shows MCPT1 and MCAD mRNA expression in NRVMs treated with ISO and a scramble siRNA (i.e., a control siRNA), and NRVMs treated with ISO and a Cyclin D2 siRNA. NRVMs treated with ISO and Cyclin D2 siRNA (i.e., siRNA D1, siRNA D2, and siRNA D3) showed mRNA expression for MCPT1 and MCAD statistically significantly greater than cardiomyocytes treated with ISO and a scramble siRNA. Overall, FIG. 8 shows that when cells are under conditions of pathologic stress (e.g., ISO-induced pathological stress), treating the cells with a Cyclin D2 oligonucleotide inhibitors (e.g., a Cyclin D2 siRNA) enhances expression of genes required for FAO in NRVMs.

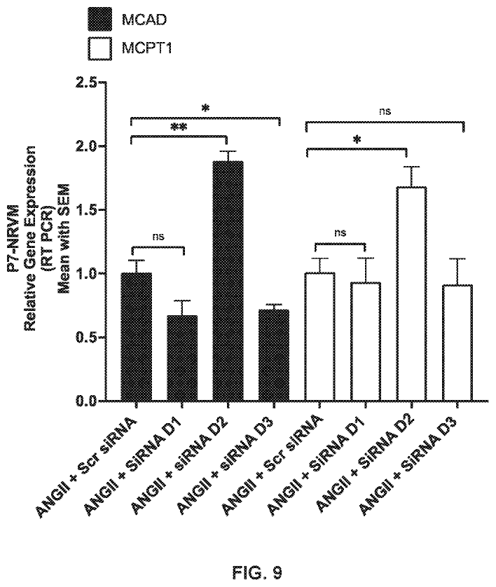

FIG. 9 shows MCPT1 and MCAD mRNA expression in NRVMs treated with ANGII and a scramble siRNA (i.e., a control siRNA), and NRVMs treated with ANGII and a Cyclin D2 siRNA. NRVMs treated with ISO and a Cyclin D2 siRNA (i.e., siRNA D1, siRNA D2, and siRNA D3) showed mRNA expression for MCPT1 and MCAD statistically significantly greater than cardiomyocytes treated with ISO and a scramble siRNA. Overall, FIG. 9 shows that when cell are under conditions of pathologic stress (e.g., ANGII-induced pathological stress), treating the cells with a Cyclin D2 oligonucleotide inhibitors (e.g., a Cyclin D2 siRNA) enhances expression of genes required for FAO in NRVMs.

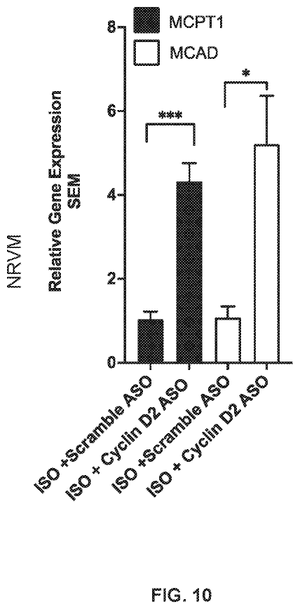

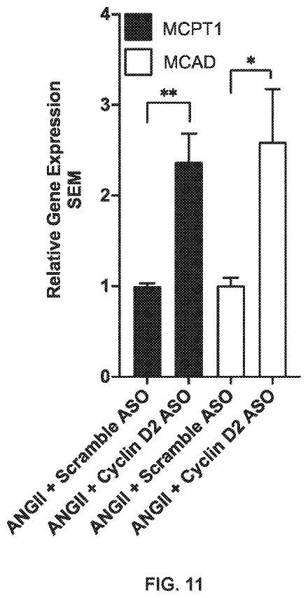

FIG. 10 shows MCPT1 and MCAD mRNA expression in NRVMs treated with ISO and a scramble ASO (i.e., a control ASO), and NRVMs treated with ISO and a Cyclin D2 ASO. NRVMs treated with ISO and a Cyclin D2 siRNA showed mRNA expression for MCPT1 and MCAD statistically significantly greater than cardiomyocytes treated with ISO and a scramble siRNA. Overall, FIG. 10 shows that when cells are under conditions of pathologic stress (e.g., ISO-induced pathological stress), treating the cells with a Cyclin D2 oligonucleotide inhibitor (e.g., an ASO having the sequence of SEQ ID NO: 6) enhances expression of genes required for FAO in NRVMs.

FIG. 11 shows MCPT1 and MCAD mRNA expression in NRVMs treated with ANGII and a scramble ASO (i.e., a control ASO), and NRVMs treated with ANGII and a Cyclin D2 ASO. NRVMs treated with ANGII and a Cyclin D2 ASO showed mRNA expression for MCPT1 and MCAD statistically significantly greater than cardiomyocytes treated with ANGII and a scramble siRNA. Overall, FIG. 11 shows that when cells are under conditions of pathologic stress (e.g., ANGII-induced pathological stress), treating the cells with a Cyclin D2 oligonucleotide inhibitor (e.g., an ASO having the sequence of SEQ ID NO: 6) enhances expression of genes required for FAO in NRVMs.

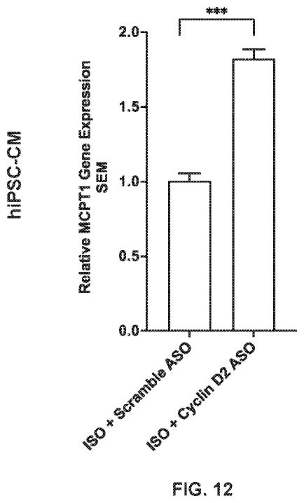

FIG. 12 shows MCPT1 mRNA expression in hiPSC-CMs treated with ISO and a Scramble ASO (i.e., a control ASO), and NRVMs treated with ISO and a Cyclin D2 ASO. hiPSC-CMs treated with ISO and a Cyclin D2 ASO showed mRNA expression for MCPT1 statistically significantly greater than cardiomyocytes treated with ISO and a scramble ASO. Overall, FIG. 12 shows that when cells are under conditions of pathologic stress (e.g., ISO-induced pathological stress), treating the cells with a Cyclin D2 oligonucleotide inhibitor (e.g., an ASO having the sequence of SEQ ID NO: 5)) enhances expression of genes required for FAO in hiPSC-CMs.

Overall, this data suggests Cyclin D2 may regulate FAO in cardiomyocytes and inhibiting or reducing Cyclin D2 is a viable therapeutic strategy for preserving and/or enhancing FAO, thereby preventing metabolic remodeling in heart failure.

To further evaluate the role of Cyclin D2 in FAO of cardiomyocytes, NRVMs were assessed for Maximal Respiration and Spare Respiratory Capacity (SRC) in response to pathological stress. SRC characterizes the mitochondrial capacity (e.g., Oxidative Phosphorylation) of a cell to meet extra energy requirements, beyond the basal level, in response to acute cellular stress or heavy workload and thereby avoiding an ATP crisis. These experiments utilized a Seahorse assay that evaluates the ability for cardiomyocytes to achieve FAO when palmitate (a fatty acid) was the primary energy source (e.g., a palmitate stress test) by measuring Oxygen Consumption Rate (OCR) of the cells.

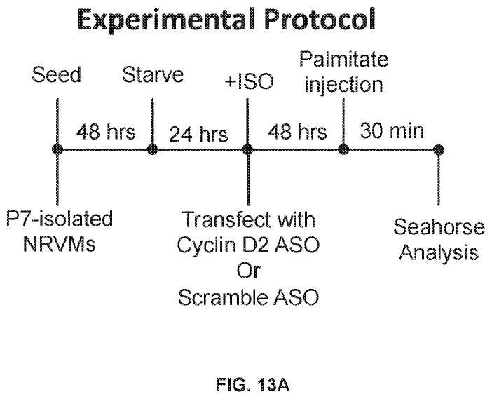

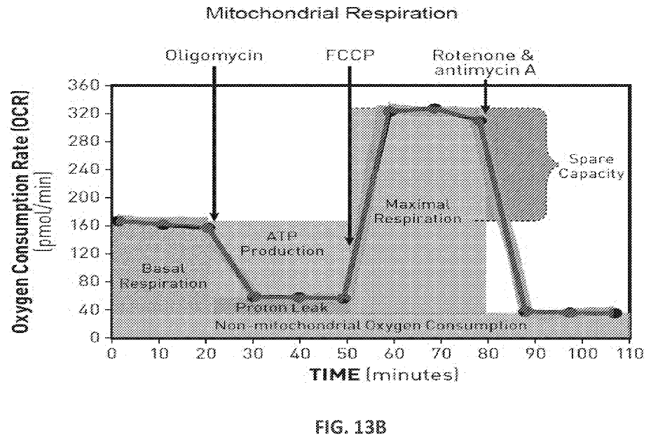

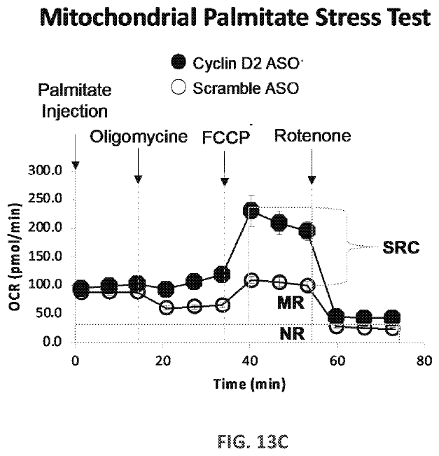

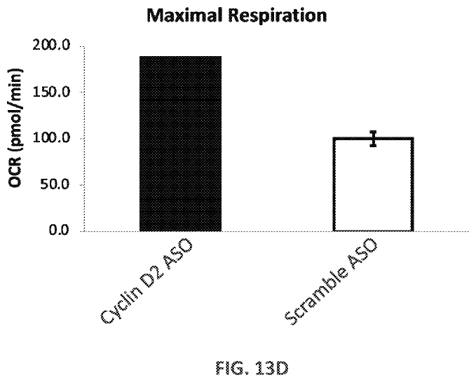

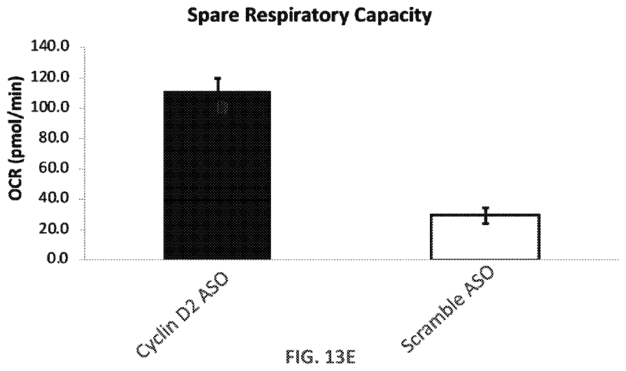

FIG. 13A shows the experimental workflow for analyzing the effect of Cyclin D2 inhibitors on FAO in cardiomyocytes in response to ISO-induced pathological stress. A guide for interpreting the experimental data generated by a Seahorse is described in FIG. 13B. The results for the Maximum Respiration and SRC analysis are provided in FIGS. 13C-13E. Data is shown as Oxygen Consumption Ratio (OCR) (pmol/min).

FIG. 13C shows OCR in NRVMs treated with ISO and transfected with scramble ASO (open circles) or Cyclin D2 ASO (black squares). NRVMs transfected with Cyclin D2 ASO showed a greater maximal respiration (FIG. 13D) and a greater Spare Reserve Capacity (FIG. 13E) in response to treatment with ISO then the cardiomyocytes transfected with scramble ASO only.

Overall, FIGS. 13C-13E show that when cells are under conditions of pathologic stress (i.e., ISO-induced pathological stress), treating the cells with a Cyclin D2 oligonucleotide inhibitor (e.g., an ASO having the sequence of SEQ ID NO: 5) enhances cardiomyocyte FAO.

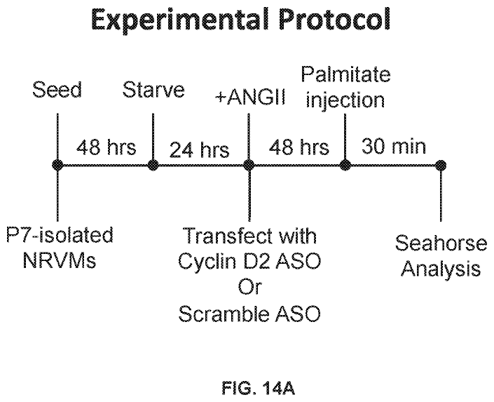

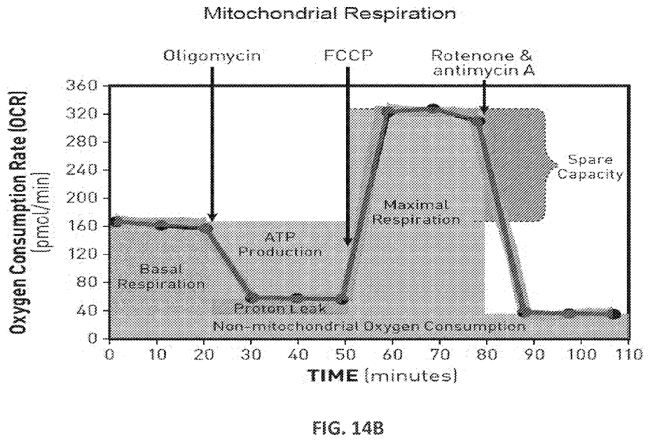

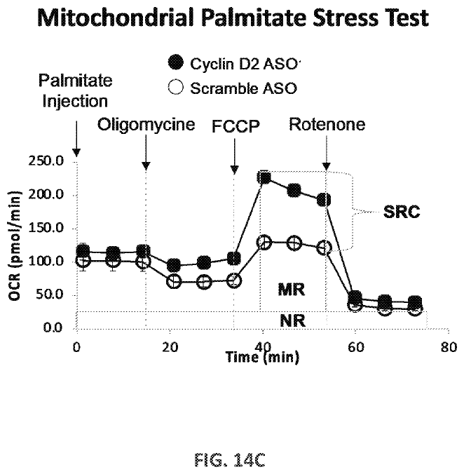

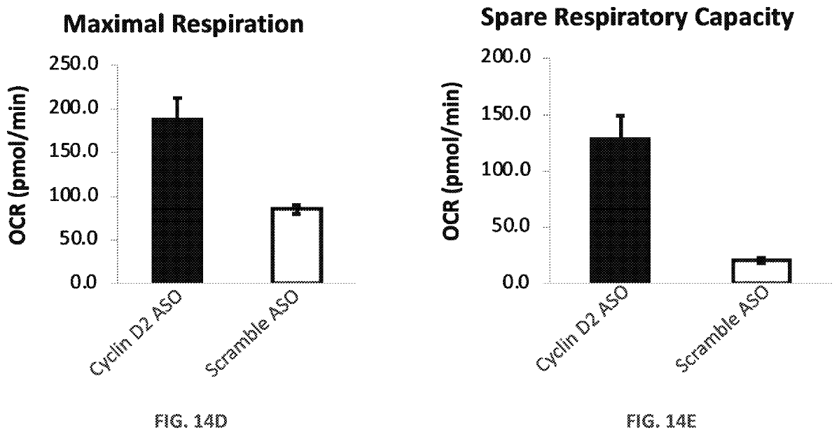

FIG. 14A shows the experimental workflow for analyzing the effect of Cyclin D2 inhibitors on FAO in cardiomyocytes in response to ANGII-induced pathological stress. A guide for interpreting the experimental data generated by Seahorse is described in FIG. 14B.

The results for the Maximum Respiration and SRC analysis are provided in FIGS. 14C-14E. Data is shown as Oxygen Consumption Ratio (OCR) (pmol/min).

FIG. 14C shows OCR in NRVMs treated with ANGII and transfected with scramble ASO (open circles) or Cyclin D2 ASO (black squares). NRVMs transfected with Cyclin D2 ASO showed a greater maximal respiration (FIG. 14D) and a greater Spare Reserve Capacity (FIG. 14E) in response to treatment with ANGII then the cardiomyocytes transfected with scramble ASO only.

Overall, FIGS. 14C-14E show that when cells are under conditions of pathologic stress (i.e., ANGII-induced pathological stress), treating the cells with a Cyclin D2 oligonucleotide inhibitor (e.g., an ASO having the sequence of SEQ ID NO: 5) enhances cardiomyocyte FAO.

Overall, these results indicate that, suppression of Cyclin D2 upregulates the expression of genes required for FAO in cardiomyocytes and increases the ability of cardiomyocytes to utilize FAO to generate ATP in response to pathologic stress. These data suggest reducing or inhibiting the expression Cyclin D2 by using an oligonucleotide-based inhibitor (e.g., using an ASO or an siRNA targeting Cyclin D2) is a viable therapeutic modality for mitigating metabolic remodeling during cardiac pathologic stress and treating heart failure associated with metabolic remodeling.

### 7.6. Example 5. ASO-Mediated Inhibition of Cyclin D2 Suppressed Fatty Acid-Accumulation

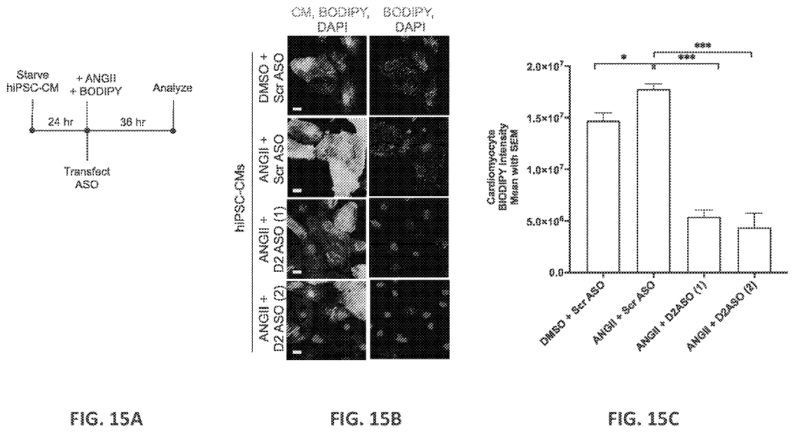

In this example, the effect of ASO-mediated Cyclin D2 suppression on fatty acid accumulation was assessed. FIG. 15A shows an example protocol for measuring the effect of Cyclin D2 suppression on fatty acid accumulation when a Cyclin D2 ASO is added at the same time as fatty acids (e.g., fatty acids in the form of BODIPY-Palmitate (BODIPY C16)). BODIPY-Palmitate (BODIPY C16) is a fluorescently (488) tagged fatty acid that was added to culture media. Pathologic stress (i.e., ANGII-induced pathological stress) increased palmitate accumulation in cardiomyocytes. Treating with a Cyclin D2 oligonucleotide inhibitor (e.g., an ASO having a sequence of SEQ ID NO:5 or SEQ ID NO:7) suppressed fatty acid accumulation in cardiomyocytes (FIGS. 15B and 15C). Overall, FIG. 15C shows that reducing or inhibiting expression of Cyclin D2 (e.g., using an ASO) suppresses cytosolic accumulation of fatty acids in cardiomyocytes.

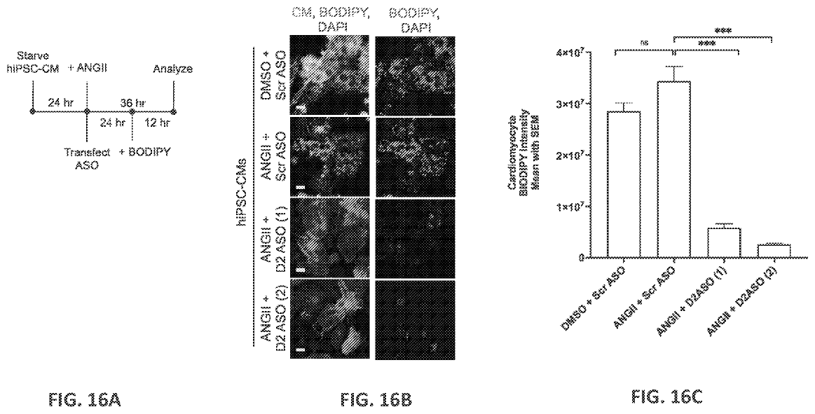

Another experiment was performed to assess the effect of ASO-mediated Cyclin D2 suppression on fatty acid accumulation when the ASO was added prophylactically (e.g., prior to the addition of the fatty acids to the culture media). FIG. 16A shows a protocol for measuring the effect of Cyclin D2 suppression on fatty acid accumulation when Cyclin D2 ASO is added before the addition of Fatty Acids (e.g., fatty acids in the form of BODIPY-Palmitate (BODIPY C16)). In particular, BODIPY-Palmitate (BODIPY C16) is a fluorescently (488) tagged fatty acid that was added to culture media after the cells were transfected with the Cyclin D2 ASO. As noted above, pathologic stress (e.g., ANGII-induced pathological stress) increases palmitate accumulation in cardiomyocytes. As shown in FIGS. 16B-16C, treating the cells with a Cyclin D2 oligonucleotide inhibitor (e.g., an ASO having a sequence of SEQ ID NO:5 or SEQ ID NO:7) suppress fatty acid accumulation in cardiomyocytes.

Overall, these results indicate that suppression of Cyclin D2 reduces lipid accumulation (thereby reducing lipotoxicity) in cardiomyocytes in response to pathologic stress. These data suggest reducing or inhibiting the expression Cyclin D2 (e.g., using a Cyclin D2 ASO) is a viable therapeutic modality for reducing lipid accumulation during cardiac pathologic stress.

### 7.7. Example 6. ASO-Mediated Inhibition of Cyclin D2 Suppressed Fatty Acid-Induced Cardiomyocyte Lipotoxicity

In this example, the effect of ASO-mediated Cyclin D2 suppression on fatty acid induced cardiomyocyte lipotoxicity was assessed. In these experiments, lipotoxicity was measured by measuring uptake of 7-AAD. Uptake of 7-AAD reflects the loss of cytosolic membrane integrity and is a proxy for cell death mediated by lipotoxicity.

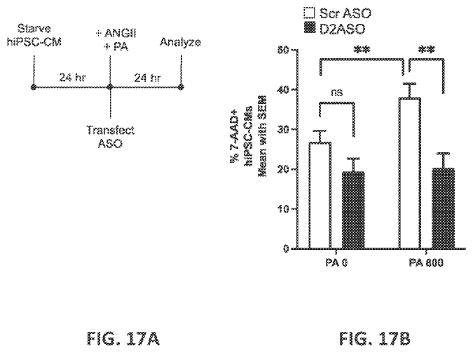

FIG. 17 shows that when hiPSC-CMs are under pathologic stress (i.e., ANGII-induced pathological stress) and high extracellular fatty acid conditions (i.e., palmitate (PA) in the culture media at 800 microMolar), treating the cells a Cyclin D2 oligonucleotide inhibitor (e.g., an ASO having the sequence of SEQ ID NO: 5) preserves hiPSC-CM viability. 7-Aminoactinomyocin D (7-ADD) is a DNA dye. 7-ADD positive cells are either apoptotic or late apoptotic/dead.

Overall, these results indicate that suppression of Cyclin D2 reduces lipotoxicity in cardiomyocytes in response to pathologic stress. These data suggest reducing or inhibiting the expression Cyclin D2 (e.g., using a Cyclin D2 ASO) is a viable therapeutic modality for reducing lipotoxicity during cardiac pathologic stress.

### Conclusion of Examples 1-5

Overall, these results indicate that Cyclin D2 oligonucleotide-based inhibitors promoted (i) suppression of hypertrophic growth, (ii) preservation, and/or stimulation, of metabolic/FAO capacity, and (iii) reduced fatty acid accumulation and lipotoxicity in cardiomyocytes.

### 8. EQUIVALENTS AND INCORPORATION BY REFERENCE

All references cited herein are incorporated by reference to the same extent as if each individual publication, database entry (e.g., Genbank sequences or GeneID entries), patent application, or patent, was specifically and individually indicated incorporated by reference in its entirety, for all purposes. This statement of incorporation by reference is intended by Applicants, pursuant to 37 C.F.R. § 1.57(b)(1), to relate to each and every individual publication, database entry (e.g., Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents. In particular, U.S. Provisional Patent Application No. 63/382,484, filed Nov. 4, 2022, is hereby incorporated by reference in its entirety.

While the invention has been particularly shown and described with reference to a preferred embodiment and various alternate embodiments, it will be understood by persons skilled in the relevant art that various changes in form and details can be made therein without departing from the spirit and scope of the invention.

9. SEQUENCE APPENDIX

SEQ ID NO: 1
>NM_001759.4 Homo sapiens cyclin D2 (CCND2), mRNA
AGAGCGGAGAAGAGCGAGCAGGGGAGAGCGAGACCAGTTTTAAGGGGAGGACCGGTGCGA
GTGAGGCAGCCCCGAGGCTCTGCTCGCCCACCACCCAATCCTCGCCTCCCTTCTGCTCCACCT
TCTCTCTCTGCCCTCACCTCTCCCCCGAAAACCCCCTATTTAGCCAAAGGAAGGAGGTCAGG
GGAACGCTCTCCCCTCCCCTTCCAAAAAACAAAAACAGAAAAACCTTTTTCCAGGCCGGGGA
AAGCAGGAGGGAGAGGGGCCGCCGGGCTGGCCATGGAGCTGCTGTGCCACGAGGTGGACCC
GGTCCGCAGGGCCGTGCGGGACCGCAACCTGCTCCGAGACGACCGCGTCCTGCAGAACCTG
CTCACCATCGAGGAGCGCTACCTTCCGCAGTGCTCCTACTTCAAGTGCGTGCAGAAGGACAT
CCAACCCTACATGCGCAGAATGGTGGCCACCTGGATGCTGGAGGTCTGTGAGGAACAGAAG
TGCGAAGAAGAGGTCTTCCCTCTGGCCATGAATTACCTGGACCGTTTCTTGGCTGGGGTCCC
GACTCCGAAGTCCCATCTGCAACTCCTGGGTGCTGTCTGCATGTTCCTGGCCTCCAAACTCAA
AGAGACCAGCCCGCTGACCGCGGAGAAGCTGTGCATTTACACCGACAACTCCATCAAGCCT
CAGGAGCTGCTGGAGTGGGAACTGGTGGTGCTGGGGAAGTTGAAGTGGAACCTGGCAGCTG
TCACTCCTCATGACTTCATTGAGCACATCTTGCGCAAGCTGCCCCAGCAGCGGGAGAAGCTG
TCTCTGATCCGCAAGCATGCTCAGACCTTCATTGCTCTGTGTGCCACCGACTTTAAGTTTGCC
ATGTACCCACCGTCGATGATCGCAACTGGAAGTGTGGGAGCAGCCATCTGTGGGCTCCAGCA
GGATGAGGAAGTGAGCTCGCTCACTTGTGATGCCCTGACTGAGCTGCTGGCTAAGATCACCA
ACACAGACGTGGATTGTCTCAAAGCTTGCCAGGAGCAGATTGAGGCGGTGCTCCTCAATAGC
CTGCAGCAGTACCGTCAGGACCAACGTGACGGATCCAAGTCGGAGGATGAACTGGACCAAG
CCAGCACCCCTACAGACGTGCGGGATATCGACCTGTGAGGATGCCAGTTGGGCCGAAAGAG
AGAGACGCGTCCATAATCTGGTCTCTTCTTCTTTCTGGTTGTTTTTGTTCTTTGTGTTTTAGGG
TGAAACTTAAAAAAAAAATTCTGCCCCCACCTAGATCATATTTAAAGATCTTTTAGAAGTGA
GAGAAAAAGGTCCTACGAAAACGGAATAATAAAAAGCATTTGGTGCCTATTTGAAGTACAG
CATAAGGGAATCCCTTGTATATGCGAACAGTTATTGTTTGATTATGTAAAAGTAATAGTAAA
ATGCTTACAGGAAAACCTGCAGAGTAGTTAGAGAATATGTATGCCTGCAATATGGGAACAA
ATTAGAGGAGACTTTTTTTTTTCATGTTATGAGCTAGCACATACACCCCCTTGTAGTATAATT
TCAAGGAACTGTGTACGCCATTTATGGCATGATTAGATTGCAAAGCAATGAACTCAAGAAG
GAATTGAAATAAGGAGGGACATGATGGGGAAGGAGTACAAAACAATCTCTCAACATGATTG
AACCATTTGGGATGGAGAAGCACCTTTGCTCTCAGCCACCTGTTACTAAGTCAGGAGTGTAG
TTGGATCTCTACATTAATGTCCTCTTGCTGTCTACAGTAGCTGCTACCTAAAAAAAGATGTTT
TATTTTGCCAGTTGGACACAGGTGATTGGCTCCTGGGTTTCATGTTCTGTGACATCCTGCTTC
TTCTTCCAAATGCAGTTCATTGCAGACACCACCATATTGCTATCTAATGGGGAAATGTAGCT
ATGGGCCATAACCAAAACTCACATGAAACGGAGGCAGATGGAGACCAAGGGTGGGATCCA
GAATGGAGTCTTTTCTGTTATTGTATTTAAAAGGGTAATGTGGCCTTGGCATTTCTTCTTAGA
AAAAAACTAATTTTTGGTGCTGATTGGCATGTCTGGTTCACAGTTTAGCATTGTTATAAACCA
TTCCATTCGAAAAGCACTTTGAAAAATTGTTCCCGAGCGATAGATGGGATGGTTTATGCAAG
TCATGCTGAATACTCCTCCCCTCTTCTCTTTTGCCCCCTCCCTTCCTGCCCCCAGTCTGGGTTA
CTCTTCGCTTCTGGTATCTGGCGTTCTTTGGTACACAGTTCTGGTGTTCCTACCAGGACTCAA
GAGACACCCCTTCCTGCTGACATTCCCATCACAACATTCCTCAGACAAGCCTGTAAACTAAA
ATCTGTTACCATTCTGATGGCACAGAAGGATCTTAATTCCCATCTCTATACTTCTCCTTTGGA
CATGGAAAGAAAAGTTATTGCTGGTGCAAAGATAGATGGCTGAACATCAGGGTGTGGCATT
TTGTTCCCTTTTCCGTTTTTTTTTTTTTATTGTTGTTGTTAATTTTATTGCAAAGTTGTATTCAG
CGTACTTGAATTTTTCTTCCTCTCCACTTCTTAGAGGCATTCAGTTAGCAAAGAGGTTGGAGC
AACAACTTTTTTTTTTTTTTTTGCACAATTGTAATTGACAGGTAATGAAGCTATTTGTTAAAA
TATTTGCCTTTTTAAGTAAAAAAGAAAAATCAGAACAGGGCTATTTGAAGAATTATTTTATA
CACAGATTCTGCCTTGTTTCATAGTATGAGGGTTGAAGACGGAAAACAATCTAAGGGTCTCT
CATTTTTTTAATTTTGTTTTGTTCAGTTTGGTTTTTTTTTTTTTTTGCGCTGCTAAGAAGCTAAA
GTCATCCATCCTTATTCACGTTGACAGTACCTAGCTGTAATGTTTCACAGAGTGTGCTGCTAT
TTTATAAACATTTTTATAATATATTATTTTACTGCTTAAATTCCAAGTCCTGAAGTAGATGGT
TGAGATATGAGTTCTTCGTACTGGAAAAGCCCTTCCGTAGTTTGTTTTCTTCTGGTAGCATAT
TCATGGTTGTTTTTTTTTTTCTTTTTTGGTTTTTTGGTTTTTTTTTTTTCCTCTGATCACATTCTT
CAAAGACGGAGTATTCTTTACCTCAGGTTTACTGGACAAAATCAATAACTACAAAAGGCAAT
GATTCACGCTTTTGTTTTCATAATACCTCACAACCGTACAGTTTCTGCTTGGGAGCCCATTCG
CATGAGGAATACAGAAGCAGTGTGAGCAGGGCTGACTCCCTCTCAGGTGGAAGGCAGGGCG
GTCTCACTCCCAGGGACCTTTTTGGTCATGGAGGCCATCGGGCTCCCAGTTAGACCCTGGTA
TCCTCATCATGATGGAAAAAATACATTGAACCAAGGGATCCTCCCTCCCCTTCAAGGCAGAC
GTTCAGTACAAACATTTATGCGGTAGGCTCAGATGTCGTAATTTGCACTTAGGTACCAGGTG
TCAGGAAACAGACTAAAAAGAATTCCACCAGGCTGTTTGGAGATCCTCATCTTGGAGCTTTT
TCAAAAGCGGGGCTTCATCTGCAAAGGGCCCTTTCATCTTGAAGTTTTTCCCCTCCGTCTTTC
CCCTCCCCTGGCATGGACACCTTGTGTTTAGGATCATCTCTGCAGGTTTCCTAGGTCTGAATC
TGCGAGTAGATGAACCTGCAGCAAGCAGCGTTTATGGTGCTTCCTTCTCCCTCCTCTGTCTCA
AACTGCGCAGGCAAGCACTATGCAAGCCCAGGCCCTCTGCTGAGCGGTACTAAACGGTCGG
GTTTTCAATCACACTGAATTGGCAGGATAAGAAAAATAGGTCAGATAAGTATGGGATGATA
GTTGAAGGGAGGTGAAGAGGCTGCTTCTCTACAGAGGTGAAATTCCAGATGAGTCAGTCTCT
TGGGAAGTGTGTTTAGAAGGGTTCAGGACTTTGTGAGTTAGCATGACCCTAAAATTCTAGGG
GATTTCTGGTGGGACAATGGGTGGTGAATTCTGAAGTTTTGGAGAGGGAAGTGGAGCAGCC
AGCAAGTAAGCTAGCCAGAGTTTTCTCAAGAGCCAGCTTTGCTCAGCACACTCTCCTGGGCC
CCAAGGAGTCCCACGGAATGGGGAAAGCGGGAACCCTGGAGTTCTTGGGAATCTTGGAGCC
TAAAGAGAAACCGAGGTGCAAATTCATTTCATGGTGACTGACCCTTGAGCTTAAACAGAAG
CAGCAAATGAAAGAACCGGACAAATAAGGAAGGGCACAAGCCTACCCGACTCTATTTACAG
TCTGTAACTTTCCACTCTTCCTGTAGTCCCGAGGCCCCTGGGTCCTTCTAGCTTTTCTCTTTCC
CATCCTTGGGGCCTTGTGTGATGATGGGTGTGGGGCTGCCGATGGGAAAGTCGGGGGTTGTT
AGGCTTTTCTGCCTGCTCCTGCTTAAACACAAGAAGGAATCCTGGATTTTGCCCTCTCCTTAG
CTCTTAGTCTCTTTGGTAGGAGTTTTGTTCCAGAGGAGCTCTCCCCCTTGGATTTGAACTTGC
TCTTTTTGTTGTTGTTGTTCTTTCTCTTCTTTTTCTTACCTCCCACTAAAGGGGTTCCAAATTAT
CCTGGTCTTTTTCTACCTTGTTGTGTTTCTATCTCGTCTTTACTTCCATCTGTTTGTTTTTTTCT
CCATCAGTGGGGGCCGAGTTGTTCCCCCAGCCTGCCAAATTTTGATCCTTCCCCTCTTTTGGC
CAAATCCTAGGGGGAAGAAATCCTAGTATGCCAAAAATATATGCTAAGCATAATTAAACTC
CATGCGGGTCCATAACAGCCAAGAAGCCTGCAGGAGAAAGCCAAGGGCAGTTCCCTCCGCA
GAACACCCCATGCGTGCTGAGAGGCGAGCTCCTTGAAGAAGGGGCTGTTCTTCCAGGAGGC
CTTATTTTGAACTGCCTCAGGACCCCACTGGAGAGCACAGCATGCCTTACTACTGGGTCATC
CTTGGTCTATGTGCTCTGTACTGGAGGCTCTGTTCTGCCTCTTATCAGCCAGGTCAGGGGCAC
ACATGGCTTAAGTGACAAAGCCAGAGGAGAAGACAACCCTGACAGCATCACGCTGCATCCC
ATTGCTAGCAGGATTGGCAACTCTTCAGACGGAGCTGCGCTTCCCTGCAGTCTAGCACCTCT
AGGGCCTCTCCAGACTGTGCCCTGGGAGCTCTGGGACTGAAAGGTTAAGAACATAAGGCAG
GATCAGATGACTCTCTCCAAGAGGGCAGGGGAATTTTCTCTCCATGGGCCACAGGGGACAG
GGCTGGGAGAAGAAATAGACTTGCACCTTATGTCATGTAAATAATTGATTTTCTAGTTCAAG
AAGATAATATTGGTAGTGTGGGAATTGGAGGTAGGAAGGGGAGGAAGTCTGAGTAAGCCAG
TTGGCTTCTAAGCCAAAAGGATTCCTCTTTGTTTATCTCTGAGACAGTCCAACCTTGAGAATA
GCTTTAAAAGGGAAATTAATGCTGAGATGATAAAGTCCCCTTAAGCCAACAAACCCTCTGTA
GCTATAGAATGAGTGCAGGTTTCTATTGGTGTGGACTCAGAGCAATTTACAAGAGCTGTTCA
TGCAGCCATCCATTTGTGCAAAATAGGGTAAGAAGATTCAAGAGGATATTTATTACTTCCTC
ATACCACATGGCTTTTGATGATTCTGGATTCTAAACAACCCAGAATGGTCATTTCAGGCACA
ACGATACTACATTCGTGTGTGTCTGCTTTTAAACTTGGCTGGGCTATCAGACCCTATTCTCGG
CTCAGGTTTTGAGAAGCCATCAGCAAATGTGTACGTGCATGCTGTAGCTGCAGCCTGCATCC
CTTCGCCTGCAGCCTACTTTGGGGAAATAAAGTGCCTTACTGACTGTAGCCATTACAGTATC
CAATGTCTTTTGACAGGTGCCTGTCCTTGAAAAACAAAGTTTCTATTTTTATTTTTAATTGGTT
TAGTTCTTAACTGCTGGCCAACTCTTACATCCCCAGCAAATCATCGGGCCATTGGATTTTTTC
CATTATGTTCATCACCCTTATATCATGTACCTCAGATCTCTCTCTCTCTCCTCTCTCTCAGTTA
TGTAGTTTCTTGTCTTGGACTTTTTTTTTTCTTTTCTTTTTCTTTTTTTTTTTGCTTTAAAACAAG
TGTGATGCCATATCAAGTCCATGTTATTCTCTCACAGTGTACTCTATAAGAGGTGTGGGTGTC
TGTTTGGTCAGGATGTTAGAAAGTGCTGATAAGTAGCATGATCAGTGTATGCGAAAAGGTTT
TTAGGAAGTATGGCAAAAATGTTGTATTGGCTATGATGGTGACATGATATAGTCAGCTGCCT
TTTAAGAGGTCTTATCTGTTCAGTGTTAAGTGATTTAAAAAAATAATAACCTGTTTTCTGACT
AGTTTAAAGATGGATTTGAAAATGGTTTTGAATGCAATTAGGTTATGCTATTTGGACAATAA
ACTCACCTTGACCTAAA
SEQ ID NO: 2
>Cyclin D2 mRNA (corresponds to 280..1149 or SEQ ID NO: 1)
ATGGAGCTGCTGTGCCACGAGGTGGACCCGGTCCGCAGGGCCGTGCGGGACCGCAACCTGC
TCCGAGACGACCGCGTCCTGCAGAACCTGCTCACCATCGAGGAGCGCTACCTTCCGCAGTGC
TCCTACTTCAAGTGCGTGCAGAAGGACATCCAACCCTACATGCGCAGAATGGTGGCCACCTG
GATGCTGGAGGTCTGTGAGGAACAGAAGTGCGAAGAAGAGGTCTTCCCTCTGGCCATGAAT
TACCTGGACCGTTTCTTGGCTGGGGTCCCGACTCCGAAGTCCCATCTGCAACTCCTGGGTGCT
GTCTGCATGTTCCTGGCCTCCAAACTCAAAGAGACCAGCCCGCTGACCGCGGAGAAGCTGTG
CATTTACACCGACAACTCCATCAAGCCTCAGGAGCTGCTGGAGTGGGAACTGGTGGTGCTGG
GGAAGTTGAAGTGGAACCTGGCAGCTGTCACTCCTCATGACTTCATTGAGCACATCTTGCGC
AAGCTGCCCCAGCAGCGGGAGAAGCTGTCTCTGATCCGCAAGCATGCTCAGACCTTCATTGC
TCTGTGTGCCACCGACTTTAAGTTTGCCATGTACCCACCGTCGATGATCGCAACTGGAAGTG
TGGGAGCAGCCATCTGTGGGCTCCAGCAGGATGAGGAAGTGAGCTCGCTCACTTGTGATGCC
CTGACTGAGCTGCTGGCTAAGATCACCAACACAGACGTGGATTGTCTCAAAGCTTGCCAGGA
GCAGATTGAGGCGGTGCTCCTCAATAGCCTGCAGCAGTACCGTCAGGACCAACGTGACGGA
TCCAAGTCGGAGGATGAACTGGACCAAGCCAGCACCCCTACAGACGTGCGGGATATCGACC
TGTGA
SEQ ID NO: 3
AUGGAGCUGCUGUGCCACGAGGUGGACCCGGUCCGCAGGGCCGUGCGGGACCGCAACCUG
CUCCGAGACGACCGCGUCCUGCAGAACCUGCUCACCAUCGAGGAGCGCUACCUUCCGCAG
UGCUCCUACUUCAAGUGCGUGCAGAAGGACAUCCAACCCUACAUGCGCAGAAUGGUGGCC
ACCUGGAUGCUGGAGGUCUGUGAGGAACAGAAGUGCGAAGAAGAGGUCUUCCCUCUGGC
CAUGAAUUACCUGGACCGUUUCUUGGCUGGGGUCCCGACUCCGAAGUCCCAUCUGCAACU
CCUGGGUGCUGUCUGCAUGUUCCUGGCCUCCAAACUCAAAGAGACCAGCCCGCUGACCGC
GGAGAAGCUGUGCAUUUACACCGACAACUCCAUCAAGCCUCAGGAGCUGCUGGAGUGGGA
ACUGGUGGUGCUGGGGAAGUUGAAGUGGAACCUGGCAGCUGUCACUCCUCAUGACUUCA
UUGAGCACAUCUUGCGCAAGCUGCCCCAGCAGCGGGAGAAGCUGUCUCUGAUCCGCAAGC
AUGCUCAGACCUUCAUUGCUCUGUGUGCCACCGACUUUAAGUUUGCCAUGUACCCACCGU
CGAUGAUCGCAACUGGAAGUGUGGGAGCAGCCAUCUGUGGGCUCCAGCAGGAUGAGGAA
GUGAGCUCGCUCACUUGUGAUGCCCUGACUGAGCUGCUGGCUAAGAUCACCAACACAGAC
GUGGAUUGUCUCAAAGCUUGCCAGGAGCAGAUUGAGGCGGUGCUCCUCAAUAGCCUGCA
GCAGUACCGUCAGGACCAACGUGACGGAUCCAAGUCGGAGGAUGAACUGGACCAAGCCAG
CACCCCUACAGACGUGCGGGAUAUCGACCUGUGA
SEQ ID NO: 4
>NP_001750.1 G1/S-specific cyclin-D2
MELLCHEVDPVRRAVRDRNLLRDDRVLQNLLTIEERYLPQCSYFKCVQKDIQPYMRRMVATW
MLEVCEEQKCEEEVFPLAMNYLDRFLAGVPTPKSHLQLLGAVCMFLASKLKETSPLTAEKLCIY
TDNSIKPQELLEWELVVLGKLKWNLAAVTPHDFIEHILRKLPQQREKLSLIRKHAQTFIALCATDF
KFAMYPPSMIATGSVGAAICGLQQDEEVSSLTCDALTELLAKITNTDVDCLKACQEQIEAVLLNS
LQQYRQDQRDGSKSEDELDQASTPTDVRDIDL
SEQ ID NO: 5
>ASO1 targeting Cyclin D2 (human) (“human cyclin D2 ASO1)(* indicates
phosphorothioate backbone, Bold letter indicates LNA)
5′ G*C*G*C*A*T*G*T*A*G*G*G*T*T*G*G*A*T 3′
SEQ ID NO: 6
>ASO targeting Cyclin D2 (Rat) (* indicates phosphorothioate backbone,
Bold letter indicates LNA)
5′ G*C*G*C*A*T*G*T*A*C*G*G*C*T*G*G*A*T 3′
SEQ ID NO: 7
>ASO targeting Cyclin D2 (human) (* indicates phosphorothioate backbone,
Bold letter indicates LNA)
5′ A*G*G*T*A*A*T*T*C*A*T*G*G*C*C*A*G*A 3′

1-2. (canceled)

3. A method for reducing or preventing hypertrophy of a plurality of cardiomyocytes, comprising:
contacting the cardiomyocytes with an effective amount of an oligonucleotide-based therapeutic comprising a polynucleotide sequence that is at least partially complementary to a nucleic acid sequence within a Cyclin D2 (CCND2) transcript, wherein Cyclin D2 expression in the cardiomyocytes is inhibited or reduced after contacting the cardiomyocytes, thereby reducing or preventing hypertrophy of a plurality of cardiomyocytes.

4. The method of claim 3, wherein the reducing or inhibiting of Cyclin D2 expression reduces or prevents hypertrophy of the plurality of cardiomyocytes.

5. The method of claim 4, wherein the plurality of cardiomyocytes are those of the heart of a patient.

6. The method of claim 5, wherein the patient has, or is at risk of having, heart failure, preferably wherein the heart failure comprises cardiac hypertrophy.

7. A method for maintaining or increasing utilization of fatty-acid oxidation in a cardiomyocyte, comprising:
contacting the cardiomyocyte with an effective amount of an oligonucleotide-based therapeutic comprising a polynucleotide sequence that is at least partially complementary to a nucleic acid sequence within a Cyclin D2 (CCND2) transcript, wherein Cyclin D2 expression is inhibited or reduced after contacting the cardiomyocyte, thereby maintaining or increasing utilization of fatty acid oxidation.

8. The method of claim 7, wherein reducing or inhibiting Cyclin D2 expression increases the cardiomyocyte's utilization of fatty-acid oxidation.

9. The method of claim 7, wherein the cardiomyocyte is in the heart of a patient.

10. A method for reducing or preventing fatty acid accumulation and fatty acid-induced lipotoxicity in a cardiomyocyte, comprising:
contacting the cardiomyocyte with an effective amount of an oligonucleotide-based therapeutic comprising a polynucleotide sequence that is at least partially complementary to a nucleic acid sequence within a Cyclin D2 (CCND2) transcript, wherein Cyclin D2 expression is inhibited or reduced after contacting the cardiomyocyte, thereby reducing lipid accumulation and lipotoxicity.

11. The method of claim 10, wherein reducing or inhibiting Cyclin D2 expression reduces lipid accumulation and lipotoxicity in the cardiomyocyte.

12. The method of claim 10, wherein the cardiomyocyte is in the heart of a patient.

13. A method for inhibiting or reducing Cyclin D2 expression in a cell, comprising: contacting the cell with an effective amount of an oligonucleotide-based therapeutic
comprising a polynucleotide sequence that is at least partially complementary to a nucleic acid sequence within a Cyclin D2 (CCND2) transcript, wherein Cyclin D2 expression is inhibited or reduced after contacting the cell with the polynucleotide sequence.

14. The method of claim 13, wherein the cell is a cardiomyocyte.

15. The method of claim 13, wherein the cardiomyocyte is in the heart of a patient.

16. The method of claim 3, wherein the polynucleotide sequence comprises a sequence having at least about 80%, at least about 85%, at least about 90%, at least about 95%, or 100% complementarity to the nucleic acid sequence within the Cyclin D2 transcript.

17. The method of claim 3, wherein the Cyclin D2 transcript is selected from a sequence of SEQ ID NO: 1-3.

18. The method of claim 3, wherein Cyclin D2 expression (protein/transcript) is reduced by at least 25% (e.g., at least 30%, at least 35%, at least 40%, at least 45%, at least 50%, at least 55%, at least 60%, at least 65%, at least 70%, at least 75%, at least 80%, at least 90%, at least 95% or 100%) as compared to Cyclin D2 expression (protein/transcript) in a patient not treated or cell not contacted with the oligonucleotide-based inhibitor.

19. The method of claim 3, wherein the oligonucleotide-based therapeutic is selected from: an anti-sense oligonucleotide (ASO), a short interfering RNA (siRNA), a single-stranded siRNAs (ssRNAs), a small temporal RNA (stRNA), a short hairpin RNA (shRNA), a locked nucleic acid (LNA), an antagomirs aptamer, a peptide nucleic acid (PNA), and a microRNA (miRNA).

20. The method of claim 19, wherein the oligonucleotide-based therapeutic is an anti-sense oligonucleotide (ASO).

21. The method of claim 20, wherein the ASO comprises a polynucleotide sequence having at least about 80%, at least about 85%, at least about 90%, at least about 95%, or about 100% complementarity to the nucleic acid sequence within the Cyclin D2 transcript.

22. The method of claim 21, wherein the ASO comprises the sequence selected from SEQ ID NOs: 5-7.

23-208. (canceled)

209. A method for treating a patient having, or at risk of having, heart failure, comprising:
administering to the patient a therapeutically effective amount of an oligonucleotide-based therapeutic comprising a polynucleotide sequence that is at least partially complementary to a nucleic acid sequence within a Cyclin D2 (CCND2) transcript, wherein Cyclin D2 expression in the patient is inhibited or reduced after the administration, thereby treating the patient having, or at risk of having, heart failure.

210. An oligonucleotide-based therapeutic for inhibiting or reducing Cyclin D2 expression in a cell, comprising a polynucleotide sequence that is at least partially complementary to a nucleic acid sequence within a Cyclin D2 (CCND2) transcript.

211. A pharmaceutical composition comprising the oligonucleotide-based therapeutic of claim 210.
